(19) United States
(12) Patent Application Publication
(10) Pub. No.: US 2010/0323041 A1
(43) Pub. Date: Dec. 23, 2010

(54) METHODS AND THERAPEUTIC COMPOSITIONS COMPRISING PLANT EXTRACTS FOR THE TREATMENT OF CANCER

(75) Inventor: Benoit CYR, Quebec (CA)

Correspondence Address: ARNOLD & PORTER LLP;ATTN: IP DOCKETING DEPT., 555 TWELFTH STREET, N.W., WASHINGTON, DC, 20004-1206, US

(73) Assignee: BIOPHARMACOPAE DESIGN INTERNATIONAL INC., Sainte-Foy (CA)

(21) Appl. No.: 12/762,139

(22) Filed: Apr. 16, 2010

Related U.S. Application Data
(63) Continuation of application No. 11/577,292, filed on Mar. 7, 2008, now abandoned.
Application No. 11/577,292 is a continuation-in-part of application No. 10/526,387, filed on Oct. 6, 2005, now abandoned.
(60) Provisional application No. 60/619,393, filed on Oct. 15, 2004.

(30) Foreign Application Priority Data
Aug. 30, 2002 (CA) 2400936

(51) Int. Cl. A61K 36/45; A61K 36/9068; A61K 36/15; A61K 36/28; A61P 35/00

(52) U.S. Cl. 424/732; 424/756

(57) ABSTRACT

A method of treating cancer by targeting two proteases, MMP-9 and cathepsin B is provided. Therapeutic compositions comprising one or more plant extracts that inhibit MMP-9 and/or cathepsin B, which are capable of inhibiting neoplastic and/or endothelial cell migration, tumor growth, tumor-induced angiogenesis and/or metastasis are also provided. The therapeutic compositions of the invention can be used in the treatment of cancer, and methods of inhibiting tumor growth, tumor metastasis, and/or tumor-induced angiogenesis using the therapeutic compositions alone or in combination with an anti-cancer agent are, therefore, also provided.

### CROSS-REFERENCE TO RELATED APPLICATIONS

This application is a continuation of U.S. Non-Provisional application Ser. No. 11/577,292, assigned a 35 U.S.C. §371 date of Mar. 7, 2008, currently pending, which is a national phase application under 35 U.S.C. §371 of International Application No. PCT/CA2005/001576, filed Oct. 17, 2005, which claims priority to and the benefit under 35 U.S.C. §119 of U.S. Provisional Application No. 60/619,393, filed Oct. 15, 2004; this application is also a continuation-in-part of U.S. patent application Ser. No. 10/526,387, filed Sep. 2, 2003, which is a national phase application under 35 U.S.C. §371 of International Application No. PCT/CA03/01284, filed Sep. 2, 2003, which claims priority to and the benefit of Canadian Patent Application No. 2,400,936, filed Aug. 30, 2002.

### FIELD OF THE INVENTION

The invention pertains to the field of cancer therapy, and in particular to the field of pharmaceutical and naturopathic compositions for the treatment of cancer.

### BACKGROUND OF THE INVENTION

Cancer is a general term frequently used to indicate any of the various types of malignant neoplasms (i.e. abnormal tissue that grows by cellular proliferation more rapidly than normal), most of which invade surrounding tissue, may metastasize to several sites, are likely to recur after attempted removal, and cause death unless adequately treated (Stedman's Medical Dictionary, Williams & Wilkins, Baltimore, Md., 26th ed. 1995). Although a variety of approaches to cancer therapy, including surgical resection, radiotherapy, and chemotherapy, have been available and commonly used for many years, cancer remains one of the leading causes of death in the world.

A large number of chemotherapeutics have been developed, however, many of these are associated with undesirable side-effects. In addition, in some cases, specific patient subgroups, such as elderly patients and patients suffering from obesity or neutropenia, exhibit an intolerance for standard/optimal chemotherapeutic doses and as a result receive sub-optimal doses of chemotherapeutics during cancer treatments (Griggs J J, Sorbero M E S, Lyman G H (2005), Arch. Inter. Med., 165(11):1267-73; Colleoni M, Gelber R D et al. (2005), Lancet, 366(9491):1108-10, Madarnas Y, et al. (2001), Breast Cancer Res Treat, 66(2):123-33, and Lyman G H, Dale D C, Crawford J. (2003), J. Clin. Oncol., 21(24):4524-31). As demonstrated by Griggs et al., administration of these sub-optimal doses of chemotherapeutics to obese women afflicted with breast cancer resulted in a poor outcome. In this case optimal doses, which were based on the patient's body size, could not be administered to overweight individuals in light of the toxic effects associated with the high doses on organs. Currently, higher chemotherapeutic dosing may be facilitated by administration of the adjuvant Neupogen®. Here, faster recovery of white blood cells may permit a patient to withstand a higher dose of chemotherapy.

Extracellular proteases (EPs), such as the serine proteases, the cathepsins, and the matrix metallo-proteases (MMPs), are believed to play several roles in the promotion of tumor growth. EPs are known to regulate the turnover of extracellular matrix (ECM) macromolecules, including collagens and glycosaminoglycans, which is important for a variety of biological processes such as angiogenesis, leukocyte or cancer cell migration and tumor invasion. EPs are also implicated in the secretion and activation of growth factors that promote tumor growth. In addition, the secretion of EPs is thought to be important for breakdown of the ECM in the tissue immediately surrounding a tumor allowing for the expansion of the tumor (Liotta L A et al: Nature 1980 Mar. 6; 284 (5751): 67-8), and certain EPs are required in the generation of new blood vessels, which are required by developing tumors to carry oxygen, waste products and growth factors, and contribute to tumor growth.

Once tumors have grown and become vascularized, they also have the potential to establish themselves at sites distant from the initial tumor, a complex multi-step process known as metastasis.

To successfully metastasize, neoplastic cells must migrate from the primary tumor mass and through tissue barriers. This involves cell locomotion from the primary to the interstitial stroma, with penetration and proteolysis of matrix material. EPs are thought to contribute to this process.

Upregulation of some MMPs has been observed in certain cancers. For example, MMP-9 has been shown to be overexpressed in advanced stage melanoma cells (MacDougall et al. Cancer Res 55: 4174-4181, 1995). Cathepsin B levels have also been found to be higher in tumors than in non-malignant tissues of the same type (Murnane et al, Cancer Res. 1991; 51:1137-42). In addition, cathepsin B expression has been found to correlate with tumor grade and lymph node metastases, as well as with overall survival and disease recurrence in some tumors (Plebani et al., Cancer, 1995, 786:367-75). For instance, gastric carcinoma with metastatic spread exhibited higher levels of cathepsin B than carcinomas without metastasis. However, in pancreatic tumours, cathepsin B overexpression appears to relate to invasive behaviour but not to metastatic spread (Ohta et al, Br. J Cancer, 1994; 69: 152-6).

Although the exact role of MMPs and cathepsins in cancer development is unclear, it has been suggested that inhibitors of individual EPs, such as MMP-9 or cathepsin B, may represent a novel therapy for cancer. Several synthetic MMP inhibitors have been developed for potential use in the treatment of cancer, examples include marimastat, prinomastat, tanomastat or metastat. However, these drugs have not yet passed beyond Phase III clinical studies in patients with advanced cancer.

To date, no synthetic or natural inhibitors of cathepsin B have reached clinical trials. A few synthetic inhibitors initially thought to have potential therapeutic benefit have been discovered, such as E-64, a potent irreversible inhibitor of cysteine proteinases, and CA-074 methyl-ester, a more selective cathepsin B inhibitor. However, these inhibitors have not been further developed for clinical use, due to reasons such as lack of substrate specificity, or irreversible inhibition profile. Leupeptin, a non-selective inhibitor of cathepsin B, has been administered with doxorubicin to treat tumours in animals (Leto et al. Anticancer Res., 50:6278, 1990). Leupeptin has also been combined with cystatin C (an endogenous molecule) in glioblastoma in mice (Konduri et al., Oncogene 21:8705). A cyclic peroxide (1-Phenyl-1,4-epoxy-1H,4H-naphtho[1,8-de][1, 2]dioxepin; ANO-2) inhibitor of urokinase-type plasminogen activator (u-PA) and cathepsin B has also recently been discovered (Arakawa et al, Int. J. Cancer 2002 Jul. 10:100(2) 220-7) and showed promising activity in a Lewis lung carcinoma model. Despite these results, further investigations of these drugs have apparently not been pursued.

Inhibitors of MMPs, including MMP-9, have been extracted from plants. For example, Sazuka et al, (1997) Biosci. Biotechnol. Biochem., 61: 1504-1506, reports the inhibition of gelatinases (MMP-2 and MMP-9) and metastasis by compounds isolated from green and black teas. Kumagai et al, JP 08104628 A2, Apr. 1, 1996 (CA 125: 67741) reports the use of flavones and anthocyanins isolated from Scutellaris baicanlensis roots to inhibit collagenase (an MMP). Dubois et al., (1998) FEBS Lett., 427: 275-278, reports the increased secretion of deleterious gelatinase-B (MMP-9) by some plant lectins. Nagase et al., (1998) Planta Med., 64: 216-219, reports the weak inhibition of collagenase by delphinidin, a flavonoid isolated from Solanum melongena.

The use of plant extracts or components of plant extracts for the treatment of cancer or for inhibiting angiogenesis has been described. For example, U.S. Pat. No. 6,649,650 describes a synergistic composition of lignans obtained from the plant extract of Cedrus deodra that exhibit anticancer activities for breast, cervix, neuroblastoma, colon, liver, lung, mouth, ovary and prostate cancer. U.S. Pat. No. 6,632,798 describes plant extracts comprising oleouropein to inhibit angiogenesis. U.S. Patent Application No. 2004/0009239 discloses herbal plant extracts of the Anoectochilus family of plants and in particular Anoectochilus formosanus, and their use for chemo-prevention, or complementary/alternative control of various human malignant diseases. U.S. Patent Application No. 2003/0171334 discloses plant extracts comprising a chemical agent of the diterpene family obtained from a member of the Euphorbiaceae family of plants for use in the treatment or prophylaxis of prostate cancer or a related cancer or condition. U.S. Patent Application No. 2003/0118677 describes plant extracts from Euphorbaciae obesa and their use for inducing apoptosis and growth inhibition of a cancerous cell.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the invention is to provide methods and therapeutic compositions comprising plant extracts for the treatment of cancer. One aspect of the present invention provides methods of attenuating tumour growth and/or metastasis by simultaneously inhibiting the activity of MMP-9 and cathepsin B.

In accordance with one aspect of the present invention, there is provided a composition for inhibition of MMP-9 and cathepsin B activity, the composition comprising one or more plant extracts capable of inhibiting MMP-9 and/or cathepsin B activity and a physiologically acceptable carrier, wherein the composition inhibits one or more of neoplastic cell migration, endothelial cell migration, tumour growth, tumour metastasis, and tumour-induced angiogenesis.

In accordance with another aspect, there is provided a use of an effective amount of a composition of the invention for inhibiting tumour growth in a subject.

In accordance with another aspect, there is provided a use of an effective amount of a composition of the invention for inhibiting tumour metastasis in a subject.

In accordance with another aspect, there is provided a use of an effective amount of a composition of the invention for inhibiting tumour-induced angiogenesis in a subject.

In accordance with another aspect, there is provided a use of a composition of the invention in the manufacture of a medicament for treating cancer in a subject.

In accordance with another aspect, there is provided a use of a composition of the invention in the manufacture of a nutraceutical for treating cancer in a subject.

In accordance with another aspect, there is provided a kit comprising a composition of the invention, at least one container, and optionally instructions for use.

In accordance with another aspect, there is provided a kit comprising a composition of the invention, and one or more anti-cancer therapeutics.

In accordance with another aspect, there is provided a method of treating cancer in a subject comprising administering to the subject an effective amount of a composition of the invention.

In accordance with another aspect of the present invention, there is provided a composition for use as an adjuvant to a chemotherapeutic in the treatment of cancer in a subject, the composition comprising one or more plant extracts capable of inhibiting MMP-9 and/or cathepsin B activity and a physiologically acceptable carrier, wherein the composition potentiates a therapeutic effect of the chemotherapeutic.

### BRIEF DESCRIPTION OF THE FIGURES

FIG. 1 presents an overview of a procedure that can be followed in accordance with one embodiment of the invention in order to generate plant extracts, each of which is derived from solid plant material.

FIG. 2 presents an overview of a procedure that can be followed in accordance with another embodiment of the present invention in order to generate plant extracts, each of which is derived from solid plant material.

FIG. 3 describes in further detail, the procedure of FIG. 1.

FIG. 4 describes in further detail, the procedure of FIG. 2.

FIG. 5 presents an overview of a commercial procedure that can be followed to prepare plant extracts based on the procedure of FIG. 1.

FIG. 6 depicts the effects of an extract from Iberis sempervirens on neoplastic cell migration (A) untreated control cells; (B) cells treated with an Iberis sempervirens extract having a concentration of 0.5×; (C) cells treated with an Iberis sempervirens extract having a concentration of 1×.

FIG. 7 depicts the anti-angiogenic effect of plant extracts of the invention in a HUVEC cellular model, (A) negative control (vehicle); (B) positive control GM-6001 (25 μg/mL); (C) positive control Fumagilin (15 μg/mL), and (D) plant extract B (10 μg/mL).

FIG. 8 depicts the anti-invasion effect of plant extracts of the invention in a tumour cell model, (A) invasive cells (MDA-MD231); (B) non-invasive cells (MCF7); and (C) plant extract A (50 μg/mL).

FIG. 9 depicts the effects of plant extracts of the invention in combination with cisplatin in the mouse Lewis lung carcinoma model of metastasis.

FIG. 10 depicts the body weight change of mice treated with plant extracts of the invention in combination with cisplatin (Lewis lung carcinoma model).

FIG. 11 depicts the effect of plant extracts of the invention alone and in combination with doxorubicin on tumour volume in a mouse melanoma model of tumour growth.

FIG. 12 depicts the effect of plant extracts of the invention alone and in combination with doxorubicin on percentage growth of tumours in a mouse melanoma model of tumour growth.

### DETAILED DESCRIPTION OF THE INVENTION

The present invention is directed to the treatment of cancer through the simultaneous targeting of two proteases, matrix metalloprotease 9 (MMP-9) and cathepsin B. As demonstrated herein, the combined targeting of these two proteases is effective in the inhibition of one or more of neoplastic cell migration, endothelial cell migration, tumour growth, tumour-induced angiogenesis and tumour metastasis. Accordingly, the present invention provides for therapeutic compositions capable of the simultaneous inhibition of MMP-9 and cathepsin B. The therapeutic compositions of the invention may be formulated as phytoceuticals, nutraceuticals or medicaments, which may be administered in accordance with conventional treatment programs, naturopathic treatment programs, and/or nutritional/supplemental programs. The invention further provides for a strategy for the treatment of cancer that involves the combined inhibition of MMP-9 and cathepsin B activity in a subject. Accordingly, there is provided a method of inhibiting tumour growth, tumour-induced angiogenesis and/or metastasis in a subject by administering to the subject effective amounts of a MMP-9 inhibitor and a cathepsin B inhibitor.

The therapeutic compositions of the invention comprise one or more plant extracts, or semi-purified/purified compound(s) prepared from plant extracts, and are capable of inhibiting MMP-9 and cathepsin B. The therapeutic compositions can comprise a single plant extract that is capable of inhibiting MMP-9, or cathepsin B, or both, or the composition can comprise two or more plant extracts, each plant extract capable of inhibiting MMP-9, or cathepsin B, or both. The compositions can further comprise one or more synthetic inhibitor, each capable of inhibiting MMP-9, or cathepsin B, or both.

The therapeutic compositions of the invention are capable of inhibiting one or more of neoplastic cell migration, endothelial cell migration, tumour growth, tumour-induced angiogenesis and metastasis. The therapeutic compositions, therefore, can be used in the treatment of cancer where inhibition of tumour growth, metastasis of tumours and/or tumour-induced angiogenesis in vivo, is desired. The present invention contemplates that the therapeutic compositions can be administered to a mammal having early stage cancer to help attenuate the progression of the disease through their effect on tumour growth and/or metastasis. It is also contemplated that the compositions can be administered prophylactically to subjects at high risk of developing a tumour, or shortly after primary therapy to prevent recurrence of a cancer. The compositions are also suitable for administration to a mammal having an advanced cancer. For example, the effects of the therapeutic compositions can lead to a weakening of the tumour, such that it is more susceptible to standard anti-cancer therapeutics.

The present invention contemplates the use of the compositions alone or in conjunction with one or more known anti-cancer therapeutics as part of a combination therapy. Therapeutic combinations of the invention may have a net therapeutic effect greater than the therapeutic effect of either the therapeutic composition or the anti-cancer therapeutic(s) of which they are comprised. The greater net therapeutic effect can be manifested, for example, as a decrease in the dose of the known anti-cancer therapeutic required to bring about a desired effect, as a decrease in the side-effects associated with the anti-cancer therapeutic(s), as a increase in the efficacy of the anti-cancer therapeutic(s), or a combination of these effects. Thus, the present invention contemplates the use of the therapeutic compositions in combination therapies wherein the standard anti-cancer therapeutic is administered at doses that are suboptimal.

Given that the therapeutic compositions of the invention may act to potentiate suboptimal doses of chemotherapeutic agent(s), use of a therapeutic composition in combination with one or more chemotherapeutic administered at sub-optimal doses for the treatment of subjects intolerant of standard chemotherapeutic, is contemplated.

### DEFINITIONS

Unless defined otherwise, all technical and scientific terms used herein have the same meaning as commonly understood by one of ordinary skill in the art to which this invention belongs.

The term “potential plants,” as used herein, is intended to include all species of the Kingdom Plantae, including terrestrial, aquatic or other plants under the Division Chlorophyta, Division Rhodophora, Division Paeophyta, Division Bryophyta and Division Tracheophyta; Subdivision Lycopsida, Subdivision Sphenopsida, Subdivision Pteropsida and Subdivision Spermopsida; Class Gymnospermae, Class Angiospermae, Subclass Dicotyledonidae and Subclass Monocotyledonidae. In general terms, plants, herbs, and lower plants such as algae are considered to be potential plants in accordance with the present invention.

The term potential plant can be used to refer to a single species of plant, or it can be used in relation to a number of closely related species of a single genus, for example, a group of closely related species that are indigenous to a certain geographical region. When a plant is identified herein by species name, it is to be understood that all varieties and hybrids of the species are encompassed by the name.

The term “plant material,” as used herein, refers to any part or parts of a plant taken either individually or in a group. Examples include, but are not limited to, leaves, flowers, roots, seeds, stems, rhizomes, tubers, and other parts of a plant, including those plants described herein as potential plants of the invention.

The term “extracellular protease” or “EP,” as used herein, refers to an enzyme that is capable of degrading proteins (i.e. proteolysis) and which is secreted outside the cell or which exerts an effect outside the cell. The cell can be prokaryotic or eukaryotic. Examples of extracellular proteases (EPs) include, but are not limited to, matrix metalloproteinases (MMPs), cathepsins, elastase, plasmin, TPA, uPA, kallikrein, ADAMS family members, neprilysin, gingipain, clostripain, thermolysin, serralysin, and other bacterial and viral proteases. While cathepsins are typically present in the lysosome, many of the cathepsins have been shown to play a role in physiological and pathological events occurring extracellularly (Reinheckel T et al: Biol Chem 2001; 382(5):735-741; Tepel C et al: J Cell Sci. 2000 December; 113 Pt 24:4487-98). Proteases such as cathepsin that exert significant effects in the extracellular matrix are, therefore, considered to be extracellular proteases in the context of the present invention. Cathepsin B and MMP-9 are extracellular proteases.

The term “panel of extracellular proteases,” refers to a plurality of distinct extracellular proteases that are used to perform routine assays to monitor the presence or absence of inhibitory activity throughout an extraction process of the invention. A panel typically comprises at least two proteases, but may for some purposes comprise as few as one protease. One skilled in the art would appreciate that as high throughput screening techniques develop, one could routinely assay for the presence or absence of inhibitory activity against as many extracellular proteases as the technology permits.

The term “plant extract,” as used herein, refers to a composition prepared by contacting plant material with a solvent following standard procedures such as those described herein. The term encompasses crude extracts, prepared by a simple extraction, as well as crude extracts that have been subjected to one or more separation and/or purification steps, including semi-purified and purified fractions and concentrates derived from a crude extract by subjecting the crude extract to one or more additional extraction, concentration, fractionation, filtration, condensation, distillation or other purification step. The plant extract may be in liquid form, such as a solution, concentrate or distillate, or it may be in solid form, such as in granulate or powder form.

The term “potential extract,” as used herein, refers to a plant extract that has not yet been determined to possess inhibitory activity against one or more extracellular protease.

The term “extract of the invention,” as used herein, refers to a plant extract that demonstrates inhibitory activity against MMP-9 and/or cathepsin B and is capable of inhibiting one or more of neoplastic cell migration, endothelial cell migration, tumour growth, tumour-induced angiogenesis and metastasis.

The term “protease inhibitor,” as used herein, refers to a plant extract or compound that attenuates the proteolytic activity of a protease. A protease inhibitor may or may not be proteinaceous.

The term “stressor,” as used herein, refers to a factor, such as a physical factor, a chemical compound, or a biological agent that is used to activate a defence response in a plant and thereby elicit production of extracellular protease inhibitors. Elicitors and inducers are also considered to be stressors.

The term “substantially purified” or “substantially pure” or “isolated,” when used in reference to a compound or compounds having protease inhibitor activity, refers to a form of the compound(s) that is relatively free of proteins, nucleic acids, lipids, carbohydrates or other materials with which it is naturally associated in a plant. As disclosed herein, a plant extract of the invention is considered to be substantially purified, in that it is removed from the plant tissue from which it is derived. In addition, compounds having protease inhibitor activity that are present within the extract can be further purified using routine and well-known methods such as those described herein. As such, a substantially pure protease inhibitor of the invention can constitute less than one percent of a sample, or it can constitute at least about one or a few percent of a sample, for example, at least about five percent of a sample. In one embodiment, the substantially pure protease inhibitor constitutes at least about twenty percent of a sample. In another embodiment, the protease inhibitor can be further purified to constitute at least about fifty percent of a sample. In a further embodiment, the protease inhibitor can be further purified to constitute at least about eighty percent of a sample. In other embodiments, the protease inhibitor can be further purified to constitute at least about ninety percent or at least about ninety-five percent or more of a sample. A determination that a protease inhibitor of the invention is substantially pure can be made using methods such as those disclosed herein or otherwise known in the art, for example, by performing electrophoresis and identifying the compound as a relatively discrete band or by performing thin layer chromatography.

The term “selective” as used herein with reference to the inhibition of an extracellular protease indicates that the plant extract, molecule or compound inhibits a selected extracellular protease with an IC₅₀ value at least one half log lower than the IC₅₀ value against other enzymes.

The terms “attenuate” and “inhibit,” as used interchangeably herein, mean to slowdown, reduce, delay or prevent.

The term “cell migration,” as used herein, refers to the movement, typically abnormal, of a cell or cells from one locus to another. Examples of cell migration include the movement of cells through the ECM or basal lamina during angiogenesis.

The terms “therapy,” and “treatment,” as used interchangeably herein, refer to an intervention performed with the intention of improving a recipient's status. The improvement can be subjective or objective and is related to the amelioration of the symptoms associated with, preventing the development of, or altering the pathology of a disease, disorder or condition being treated. Thus, the terms therapy and treatment are used in the broadest sense, and include the prevention (prophylaxis), moderation, reduction, and curing of a disease, disorder or condition at various stages. Prevention of deterioration of a recipient's status (i.e. stabilisation of the disease, disorder or condition) is also encompassed by the terms. Those in need of therapy/treatment include those already having the disease, disorder or condition as well as those prone to, or at risk of developing, the disease, disorder or condition and those in whom the disease, disorder or condition is to be prevented.

The term “nutraceutical,” as used herein, refers to a food or dietary supplement that protects or promotes health and/or provides a benefit to a subject which affects the long term health of the subject.

The term “phytoceutical,” as used herein, refers to a plant-comprising composition having therapeutic properties.

The term “phyto-synthetic composition,” as used herein, refers to a therapeutic composition of the invention that comprises one or more synthetic MMP-9 and/or cathepsin B inhibitors in addition to one or more plant-derived MMP-9 and/or cathepsin B inhibitors.

The term “adjuvant,” as used herein, refers to substance that enhances and/or potentiates the therapeutic effect of another substance (such as a chemotherapeutic drug). In contrast, the term “adjuvant therapy,” as used herein with respect to cancer therapies, refers to a therapy that follows a primary therapy and that is administered to subjects at risk of relapsing. “Primary therapy” refers to a first line of treatment upon the initial diagnosis of cancer in a subject.

The term “sub-optimal dose,” as used herein, refers to a dose below the recommended dose for a given substance (i.e. refers to a dose that is below the standard or optimal dose). In one embodiment of the present invention, a dose of a given chemotherapeutic drug is defined as sub-optimal when it is > or =5% below the standard dose for the drug at a given cycle of treatment. In another embodiment, a sub-optimal dose is defined as a dose > or =10% below the standard dose for the chemotherapeutic drug at a given cycle of treatment. In a further embodiment, a suboptimal dose is defined as a dose > or =15% below the standard dose for the chemotherapeutic drug at a given cycle of treatment.

The terms “ameliorate” or “amelioration” include the arrest, prevention, decrease, or improvement in one or more the symptoms, signs, and features of the disease, disorder or condition being treated, both temporary and long-term.

The term “subject” or “patient,” as used herein, refers to an animal in need of treatment.

The term “animal,” as used herein, refers to both human and non-human animals, including, but not limited to, mammals, birds and fish.

Administration of the composition of the invention “in combination with” one or more further therapeutic agents, is intended to include simultaneous (concurrent) administration and consecutive administration. Concurrent administration is intended to encompass administration of the therapeutic agent(s) and the composition(s) of the invention to the subject via various routes. Consecutive administration is intended to encompass administration of the therapeutic agent(s) and the composition(s) of the invention to the subject in various orders and via various routes.

As used herein, the term “about” refers to a +/−10% variation from the nominal value. It is to be understood that such a variation is always included in any given value provided herein, whether or not it is specifically referred to.

Other chemistry terms herein are used according to conventional usage in the art, as exemplified by The McGraw-Hill Dictionary of Chemical Terms (ed. Parker, S., 1985, McGraw-Hill, San Francisco).

### Therapeutic Compositions

As indicated above, the therapeutic compositions of the present invention are capable of simultaneous inhibition of two proteases, MMP-9 and cathepsin B. In accordance with the present invention, the therapeutic compositions comprise one or more plant extracts, or semi-purified/purified compound(s) prepared therefrom, that inhibit MMP-9 protease activity and/or cathepsin B protease activity. Thus, any given plant extract included in the therapeutic composition may be capable of inhibiting either MMP-9 or cathepsin B, or it capable of inhibiting both of these proteases. When the compositions comprise more than one plant extract, the plant extracts can be inhibitors of either MMP-9 or cathepsin B, or they can be a combination of MMP-9 inhibitors and cathepsin B inhibitors. In one embodiment of the invention, the compositions comprise one plant extract. In another embodiment, the compositions comprise two plant extracts. In a further embodiment, the compositions comprise two or more plant extracts. In another embodiment, the compositions comprise a combination of one or more MMP-9 inhibiting plant extract and one or more cathepsin B inhibiting plant extract.

In a specific embodiment of the invention, the therapeutic composition comprises one or more plant extracts derived from the plants set forth in Tables 6 to 9. In an alternative embodiment of the invention, the therapeutic compositions comprise at least one plant extract derived from a plant belonging to the Family Zingiberaceae, the Family Pinaceae or the Family Asteraceae. In another embodiment of the invention, the therapeutic compositions comprise at least one plant extract derived from a plant belonging to the Zingiber, Tsuga or Solidago genus of plants. In a further embodiment, the therapeutic composition comprises one or more plant extracts derived from plants selected from the group of: Zingiber officinale, Solidago sp. and Tsuga canadensis. In a further embodiment, the Solidago sp., is Solidago canadensis, Solidago gigantea (also known as Solidago serotina), Solidago virgaurea, Solidago hybrida, or a combination thereof. In another embodiment of the invention, the therapeutic composition comprises two extracts, where the plant extracts are derived from Zingiber officinale and Solidago sp.

For compositions comprising two or more plant extracts, various ratios of the constituent plant extracts are contemplated. By way of example, for a composition comprising two plant extracts, for example, extract A and extract B, the ratio of extract A to extract B can vary anywhere between 1:99 and 99:1. By “anywhere between 99:1 and 1:99” it is meant that the ratio of the two extracts can be defined by any ratio within this range, thus the ratio can be between 98:2 and about 1:99 between about 98:2 and 2:98, between 97:3 and 1:99, between 97:3 and 2:98, between 97:3 and 3:97, etc. In one embodiment of the present invention, the ratio of the two extracts is between about 90:10 and about 10:90. In another embodiment, the ratio of the two extracts is between about 80:20 and about 20:80. In a further embodiment, the ratio of the two extracts is between about 70:30 and about 30:70. In another embodiment, the ratio of the two extracts is between about 60:40 and about 40:60. In another embodiment, the ratio of the two extracts is about 50:50.

In an alternative embodiment, the ratio of the two plant extracts is between about 1:5 and about 5:1. In a further embodiment, the ratio of the two plant extracts is between about 1:4 and about 4:1. In other embodiments, the ratio of the two plant extracts is between about 1:3 and about 3:1, and between about 1:2 and about 2:1.

Analogous ratios are contemplated for compositions comprising more than two plant extracts. Thus, for example, for compositions comprising three plant extracts, extract A, extract B and extract C, the ratio of extract A to extract B to extract C can vary anywhere between 1:1:98 and 98:1:1. Likewise, for compositions comprising four plant extracts, extract A, extract B, extract C and extract D, the ratio of extract A to extract B to extract C to extract D can vary anywhere between 1:1:1:97 and 97:1:1:1. Similar ratios for compositions comprising more than four extracts can readily be envisaged.

The present invention contemplates the simultaneous targeting of two proteases, MMP-9 and cathepsin B. When a composition comprises more than one plant extract, various combinations of MMP-9 and cathepsin B inhibitors are contemplated. For example, the composition may comprise one or more extracts that inhibit MMP-9 only, plus one extract capable of inhibiting cathepsin B and/or MMP-9. Similarly, the composition may comprise one or more extracts that inhibit cathepsin B only, plus one extract capable of inhibiting MMP-9 and/or cathepsin B. Also contemplated is a composition comprising more than one plant extract where each extract is capable of inhibiting both cathepsin B and MMP-9.

The therapeutic compositions contemplated by the present invention also include phyto-synthetic compositions comprising one or more plant extracts in combination with one or more synthetic MMP-9 and/or cathepsin B inhibitors. Various MMP-9 and cathepsin B inhibitor combinations are envisioned. Thus, for example, when the plant extract(s) included in the therapeutic composition inhibits MMP-9 only, then cathepsin B inhibitory activity can be provided by including a synthetic cathepsin B inhibitor in the therapeutic composition. Similarly, when the plant extract(s) included in the therapeutic composition inhibits cathepsin B only, then MMP-9 inhibitory activity can be provided by including a synthetic MMP-9 inhibitor in the therapeutic composition. In any event, the phyto-synthetic compositions contemplated by the invention are capable of inhibiting both MMP-9 and cathepsin B and are also capable of inhibiting one or more of neoplastic cell migration, endothelial cell migration, tumour growth, tumour-induced angiogenesis and metastasis.

In one embodiment of the invention, when a composition comprises both a MMP-9 inhibitor and a cathepsin B inhibitor, either in the form of a plant extract, or compound derived therefrom, or as a synthetic inhibitor, the net therapeutic effect of the composition is greater than the therapeutic effect of either of the inhibitors alone.

The present invention further contemplates therapeutic combinations comprising a therapeutic composition in combination with one or more anti-cancer therapeutics. These therapeutic combinations can be formulated as a single pharmaceutical composition or, more typically, comprise separate compositions that are designed to be administered in combination.

### Components of the Therapeutic Compositions

### 1. Plant Extracts

Plant material suitable for preparation of a plant extract for inclusion in a therapeutic composition of the invention is derived from a “potential plant.” Plant extracts capable of inhibiting MMP-9 and/or cathepsin B have been isolated from a variety of plant species as described herein and are suitable candidate extracts for inclusion in the compositions of the invention. It will be readily apparent to one skilled in the art that other extracts capable of inhibiting MMP-9 and/or cathepsin B could be isolated using similar techniques from a wide range of plants, i.e. potential plants. Potential plants include all species of the Kingdom Plantae, including terrestrial, aquatic or other plants that can be subjected to standard extraction procedures, such as those described herein, in order to generate an extract that can be tested for its ability to inhibit MMP-9 and/or cathepsin B. Extracts demonstrating inhibitory activity against MMP-9 and/or cathepsin B are considered to be suitable candidate extracts for use in the therapeutic compositions of the invention.

Examples of potential plants include, but are not limited to, those belonging to the following classifications: Superdivision Spermatophyta—Seed plants; Division Coniferophyta—Conifers; Class Pinopsida, Order Pinales; Family Araucariaceae—Araucaria family; Family Cephalotaxaceae—Plum Yew family; Family Cupressaceae—Cypress family; Family Pinaceae—Pine family; Family Podocarpaceae—Podocarpus family; Family Taxodiaceae—Redwood family; Order Taxales, Family Taxaceae—Yew family; Division Cycadophyta—Cycads, Class Cycadopsida, Order Cycadales, Family Cycadaceae—Cycad family; Family Zamiaceae—Sago-palm family; Division Ginkgophyta—Ginkgo, Class Ginkgoopsida, Order Ginkgoales, Family Ginkgoaceae—Ginkgo family; Division Gnetophyta—Mormon tea and other gnetophytes, Class Gnetopsida, Order Ephedrales, Family Ephedraceae—Mormon-tea family; Order Gnetales, Family Gnetaceae—Gnetum family; Division Magnoliophyta—Flowering plants, Class Liliopsida—Monocotyledons, Subclass Alismatidae, Order Alismatales, Family Alismataceae—Water-plantain family, Family Butomaceae—Flowering Rush family, Family Limnocharitaceae—Water-poppy family; Order Hydrocharitales, Family Hydrocharitaceae—Tape-grass family; Order Najadales, Family Aponogetonaceae—Cape-pondweed family, Family Cymodoceaceae—Manatee-grass family, Family Juncaginaceae—Arrow-grass family, Family Najadaceae—Waternymph family, Family Posidoniaceae—Posidonia family, Family Potamogetonaceae—Pondweed family, Family Ruppiaceae—Ditch-grass family, Family Scheuchzeriaceae—Scheuchzeria family, Family Zannichelliaceae—Horned pondweed family, Family Zosteraceae—Eel-grass family; Subclass Arecidae, Order Arales, Family Acoraceae—Calamus family, Family Araceae—Arum family, Family Lemnaceae—Duckweed family; Order Arecales, Family Arecaceae—Palm family; Order Cyclanthales, Family Cyclanthaceae—Panama Hat family; Order Pandanales, Family Pandanaceae—Screwpine family; Subclass Commelimidae, Order Commelinales, Family Commelinaceae—Spiderwort family, Family Mayacaceae—Mayaca family, Family Xyridaceae—Yellow-eyed Grass family; Order Cyperales, Family Cyperaceae—Sedge family, Family Poaceae—Grass family; Order Eriocaulales, Family Eriocaulaceae—Pipewort family; Order Juncales, Family Juncaceae—Rush family; Order Restionales, Family Joinvilleaceae—Joinvillea family; Order Typhales, Family Sparganiaceae—Bur-reed family, Family Typhaceae—Cat-tail family; Subclass Liliidae, Order Liliales, Family Agavaceae—Century-plant family, Family Aloeaceae—Aloe family, Family Dioscoreaceae—Yam family, Family Haemodoraceae—Bloodwort family, Family Hanguanaceae—Hanguana family, Family Iridaceae—Iris family, Family Liliaceae—Lily family, Family Philydraceae—Philydraceae family, Family Pontederiaceae—Water-Hyacinth family, Family Smilacaceae—Catbrier family, Family Stemonaceae—Stemona family, Family Taccaceae—Tacca family; Order Orchidales, Family Burmanniaceae—Burmannia family, Family Orchidaceae—Orchid family; Subclass Zingiberidae, Order Bromeliales, Family Bromeliaceae—Bromeliad family; Order Zingiberales, Family Cannaceae—Canna family, Family Costaceae—Costus family, Family Heliconiaceae—Heliconia family, Family Marantaceae—Prayer-Plant family, Family Musaceae—Banana family, Family Zingiberaceae—Ginger family; Class Magnoliopsida—Dicotyledons, Subclass Asteridae, Order Asterales, Family Asteraceae—Aster family; Order Callitrichales, Family Callitrichaceae—Water-starwort family, Family Hippuridaceae—Mare's-tail family; Order Calycerales, Family Calyceraceae—Calycera family; Order Campanulales, Family Campanulaceae Bellflower family, Family Goodeniaceae—Goodenia family, Family Sphenocleaceae Spenoclea family; Order Dipsacales, Family Adoxaceae—Moschatel family, Family Caprifoliaceae—Honeysuckle family, Family Dipsacaceae—Teasel family, Family Valerianaceae—Valerian family; Order Gentianales, Family Apocynaceae—Dogbane family, Family Asclepiadaceae—Milkweed family, Family Gentianaceae—Gentian family, Family Loganiaceae—Logania family; Order Lamiales, Family Boraginaceae—Borage family, Family Lamiaceae—Mint family, Family Lennoaceae—Lennoa family, Family Verbenaceae—Verbena family; Order Plantaginales, Family Plantaginaceae—Plantain family; Order Rubiales, Family Rubiaceae—Madder family; Order Scrophulariales, Family Acanthaceae—Acanthus family, Family Bignoniaceae—Trumpet-creeper family, Family Buddlejaceae—Butterfly-bush family, Family Gesneriaceae—Gesneriad family, Family Lentibulariaceae—Bladderwort family, Family Myoporaceae—Myoporum family, Family Oleaceae—Olive family, Family Orobanchaceae—Broom-rape family, Family Pedaliaceae—Sesame family, Family Scrophulariaceae—Figwort family; Order Solanales, Family Convolvulaceae—Morning-glory family, Family Cuscutaceae—Dodder family, Family Fouquieriaceae—Ocotillo family, Family Hydrophyllaceae—Waterleaf family, Family Menyanthaceae—Buckbean family, Family Polemoniaceae—Phlox family, Family Solanaceae—Potato family; Subclass Caryophyllidae, Order Caryophyllales, Family Achatocarpaceae—Achatocarpus family, Family Aizoaceae—Fig-marigold family, Family Amaranthaceae—Amaranth family, Family Basellaceae—Basella family, Family Cactaceae—Cactus family, Family Caryophyllaceae—Pink family, Family Chenopodiaceae—Goosefoot family, Family Molluginaceae—Carpet-weed family, Family Nyctaginaceae—Four o'clock family, Family Phytolaccaceae—Pokeweed family, Family Portulacaceae—Purslane family; Order Plumbaginales, Family Plumbaginaceae—Leadwort family; Order Polygonales, Family Polygonaceae—Buckwheat family; Subclass Dilleniidae, Order Batales, Family Bataceae—Saltwort family; Order Capparales, Family Brassicaceae—Mustard family, Family Capparaceae—Caper family, Family Moringaceae—Horse-radish tree family, Family Resedaceae—Mignonette family; Order Diapensiales, Family Diapensiaceae—Diapensia family; Order Dilleniales, Family Dilleniaceae—Dillenia family, Family Paeoniaceae—Peony family; Order Ebenales, Family Ebenaceae—Ebony family, Family Sapotaceae—Sapodilla family, Family Styracaceae—Storax family, Family Symplocaceae—Sweetleaf family; Order Ericales, Family Clethraceae—Clethra family, Family Cyrillaceae—Cyrilla family, Family Empetraceae—Crowberry family, Family Epacridaceae—Epacris family, Family Ericaceae—Heath family, Family Monotropaceae—Indian Pipe family, Family Pyrolaceae—Shinleaf family; Order Lecythidales, Family Lecythidaceae—Brazil-nut family; Order Malvales, Family Bombacaceae—Kapok-tree family, Family Elaeocarpaceae—Elaeocarpus family, Family Malvaceae—Mallow family, Family Sterculiaceae—Cacao family, Family Tiliaceae—Linden family; Order Nepenthales, Family Droseraceae—Sundew family, Family Nepenthaceae—East Indian Pitcher-plant family, Family Sarraceniaceae—Pitcher-plant family; Order Primulales, Family Myrsinaceae—Myrsine family, Family Primulaceae—Primrose family, Family Theophrastaceae—Theophrasta family; Order Salicales, Family Salicaceae—Willow family; Order Theales, Family Actinidiaceae—Chinese Gooseberry family, Family Caryocaraceae—Souari family, Family Clusiaceae—Mangosteen family, Family Dipterocarpaceae—Meranti family, Family Elatinaceae—Waterwort family, Family Marcgraviaceae—Shingle Plant family, Family Ochnaceae—Ochna family, Family Theaceae—Tea family; Order Violales, Family Begoniaceae—Begonia family, Family Bixaceae—Lipstick-tree family, Family Caricaceae—Papaya family, Family Cistaceae—Rock-rose family, Family Cucurbitaceae—Cucumber family, Family Datiscaceae—Datisca family, Family Flacourtiaceae—Flacourtia family, Family Frankeniaceae—Frankenia family, Family Loasaceae—Loasa family, Family Passifloraceae—Passion-flower family, Family Tamaricaceae—Tamarix family, Family Turneraceae—Turnera family, Family Violaceae—Violet family; Subclass Hamamelidae, Order Casuarinales, Family Casuarinaceae—She-oak family; Order Fagales, Family Betulaceae—Birch family, Family Fagaceae—Beech family; Order Hamamelidales, Family Cercidiphyllaceae—Katsura-tree family, Family Hamamelidaceae—Witch-hazel family, Family Platanaceae—Plane-tree family; Order Juglandales, Family Juglandaceae—Walnut family; Order Leitneriales, Family Leitneriaceae—Corkwood family; Order Myricales, Family Myricaceae—Bayberry family; Order Urticales, Family Cannabaceae—Hemp family, Family Cecropiaceae—Cecropia family, Family Moraceae—Mulberry family, Family Ulmaceae—Elm family, Family Urticaceae—Nettle family; Subclass Magnoliidae, Order Aristolochiales, Family Aristolochiaceae—Birthwort family; Order Illiciales, Family Illiciaceae—Star-anise family, Family Schisandraceae—Schisandra family; Order Laurales, Family Calycanthaceae—Strawberry-shrub family, Family Hernandiaceae—Hernandia family, Family Lauraceae—Laurel family, Family Monimiaceae—Monimia family; Order Magnoliales, Family Annonaceae—Custard-apple family, Family Canellaceae—Canella family, Family Magnoliaceae—Magnolia family, Family Myristicaceae—Nutmeg family, Family Sonneratiaceae—Sonneratia family, Family Winteraceae—Wintera family; Order Nymphaeales, Family Cabombaceae—Water-shield family, Family Ceratophyllaceae—Hornwort family, Family Nelumbonaceae—Lotus-lily family, Family Nymphaeaceae—Water-lily family; Order Papaverales, Family Fumariaceae—Fumitory family, Family Papaveraceae—Poppy family; Order, Piperales, Family Chloranthaceae—Chloranthus family, Family Piperaceae—Pepper family, Family Saururaceae—Lizard's-tail family; Order Ranunculales, Family Berberidaceae—Barberry family, Family Lardizabalaceae—Lardizabala family, Family Menispermaceae—Moonseed family, Family Ranunculaceae—Buttercup family, Family Sabiaceae—Sabia family; Subclass Rosidae, Order Apiales, Family Apiaceae—Carrot family, Family Araliaceae—Ginseng family; Order Celastrales, Family Aquifoliaceae—Holly family, Family Celastraceae—Bittersweet family, Family Corynocarpaceae—Karaka family, Family Hippocrateaceae—Hippocratea family, Family Icacinaceae—Icacina family, Family Stackhousiaceae—Stackhousia family; Order Cornales, Family Cornaceae—Dogwood family, Family Garryaceae—Silk Tassel family, Family Nyssaceae—Sour Gum family; Order Euphorbiales, Family Buxaceae—Boxwood family, Family Euphorbiaceae—Spurge family, Family Simmondsiaceae—Jojoba family; Order Fabales, Family Fabaceae—Pea family; Order Geraniales, Family Balsaminaceae—Touch-me-not family, Family Geraniaceae—Geranium family, Family Limnanthaceae—Meadow-Foam family, Family Oxalidaceae—Wood-Sorrel family, Family Tropaeolaceae—Nasturtium family; Order Haloragales, Family Gunneraceae—Gunnera family, Family Haloragaceae—Water Milfoil family; Order Linales Family Erythroxylaceae—Coca family, Family Linaceae—Flax family; Order Myrtales, Family Combretaceae—Indian Almond family, Family Lythraceae—Loosestrife family, Family Melastomataceae—Melastome family, Family Myrtaceae—Myrtle family, Family Onagraceae—Evening Primrose family, Family Punicaceae—Pomegranate family, Family Thymelaeaceae—Mezereum family, Family Trapaceae—Water Chestnut family; Order Podostemales, Family Podostemaceae—River-weed family; Order Polygalales, Family Krameriaceae—Krameria family, Family Malpighiaceae—Barbados Chemy family, Family Polygalaceae—Milkwort family; Order Proteales, Family Proteaceae—Protea family; Order Rafflesiales, Family Rafflesiaceae—Rafflesia family; Order Rhamnales, Family Elaeagnaceae—Oleaster family, Family Rhamnaceae—Buckthorn family, Family Vitaceae—Grape family; Order Rhizophorales, Family Rhizophoraceae—Red Mangrove family; Order Rosales, Family Brunelliaceae—Brunellia family, Family Chrysobalanaceae—Cocoa-plum family, Family Connaraceae—Cannarus family, Family Crassulaceae—Stonecrop family, Family Crossosomataceae—Crossosoma family, Family Cunoniaceae—Cunonia family, Family Grossulariaceae—Currant family, Family Hydrangeaceae—Hydrangea family, Family Pittosporaceae—Pittosporum family Family Rosaceae—Rose family, Family Saxifragaceae—Saxifrage family, Family Surianaceae—Suriana family; Order Santalales, Family Balanophoraceae—Balanophora family, Family Eremolepidaceae—Catkin-mistletoe family, Family Loranthaceae—Showy Mistletoe family, Family Olacaceae—Olax family, Family Santalaceae—Sandalwood family, Family Viscaceae—Christmas Mistletoe family; Order Sapindales, Family Aceraceae—Maple family, Family Anacardiaceae—Sumac family, Family Burseraceae—Frankincense family, Family Hippocastanaceae—Horse-chestnut family, Family Meliaceae—Mahogany family, Family Rutaceae—Rue family, Family Sapindaceae—Soapberry family, Family Simaroubaceae—Quassia family, Family Staphyleaceae—Bladdernut family, Family Zygophyllaceae—Creosote-bush family.

Groups of potential plants may also be selected based on their indigenous geographical regions. For example, one group of potential plants could comprise plants that are indigenous to arid regions, for example, those located between 35° north latitude and 35° south latitude. In accordance with another embodiment of the present invention, therefore, potential plants comprise: the agave, Agavaceae, family including such members as: Yucca elata, Y. breviflora, Agave deserti, A. chrysantha, Dasylirion wheeleri; the buckwheat, Polygonaceae, family, such as Eriogonum fasciculatum; the crowfoot, Ranunculaceae, family, such as Delphinium scaposum, Anemone tuberosa and D. parishii; the poppy, Papaveraceae, family, including Platystemon californicus, Argemone pleiacantha, Corydalis aurea, Eschschoizia californica and Ar. corymbosa; members of the mustard, Cruciferae, family, such as Dithyrea californica, Streptanthus carinatus and Lesquerella gordoni; members of the legume, Leguminosae, family, such as Acacia greggii, Prosopis velutina, A. constrica, Senna covesii, Cercidium floridum, C. microphyllum, Lotus huminstratus, Krameria parvifolia, Parkinsonia aculeata, Calliendia eriophylla, Lupinus arizonicus, Olyneya tesota, Astragalus lentiginosus, Psorothamunus spinosus and Lupinus sparsiflorus; members of the loasa family, Loasaceae, including Mentzelia involucrata, M. pumila and Mohavea Confertiflora; members of the cactus, Cactaceae, family, such as Carnegiea gigantia, Opuntia leptocaulis, Ferocactus wislizenii, O. bigelovii, O. pheacantha, O. versicolor, O. fulgida, Echinocereus engelmannii, Mammillaria microcarpa, O. basilaris, Stenocereins thurberi, O. violacea, M. tetrancistra, O. ramosissima, O. acanthocarpa, E. pectinatins and O. arbuscula; members of the evening primrose, Onagraceae, family, such as Oenothera deltoides, Camissonia claviformis and Oe. primiveris; members of the milkweed, Asclepiadaceae, family, including Asclepias erosa, A. sublata and Sarcostemma cynanchoides; members of the borage, Boraginaceae, family, such as Cryptantha augusti folia and Amsinckia intermedia; members of the sunflower, Compositae, family, including Baccharis sarothroides, Monoptiilon belloides, Erieron divergens, Zinnia acerosa, Melampodium leucanthan, Chaenactis fremontii, Calycoseris wrightii, Malacothrix californica, Helianthus annus, H. niveus, Geraea canescens, Hymenothrix wislizenii, Encelia farinosa, Psilostrophe cooperi, Baileya multiradiata, Bebbia juncea, Senecio douglasii, Trixis californica, Machaeranthera tephrodes, Xylorhiza tortifolia, Cirsiinm neomexicanum, Antennaria parviflora and Ch. douglasii; members of the caltrop, Zygophyllaceae, family, including Larrea tridentata and Kallstroemia grandiflora; members of the mallow, Malvaceae, family, including Hibiscus coulteri, H. denudatus and Sphaeralcea ambigua; members of the phlox, Polemoniaceae, family, such as Luanthus aureus; members of the unicorn plant, Martyniaceae, family, such as Proboscidiea altheaefolia; members of the gourd, Cucurbitaceae, family, such as Cucurbita digitata; members of the lily, Lilaceae, family, including Calochortus kennedyi, Dichelostemma pulchellum, Allium macropetalum and Hesperocallis indulata; members of the ocotillo, Fouquieriaceae, family, including Fouquieria splendens; members of the figwort, Scrophulariaceae, family, such as Castilleja sp., Penstemon parryi and Orthocarpus purpurascens; members of the acanthus, Acanthaceae, family, including Anisacanthus thurberi, Justicia californica and Ruellia nudiflora; members of the four o'clock, Nyctaginaceae, family, such as Allionia incarnata, Abronia villosa and Mirabilis multiflora; members of the geranium, Geraniaceae, family, including Erodium cicutarium; members of the waterleaf, Hydrophyllaceae, family, such as Nama demissum, Phacelia bombycina and Ph. distans; members of the bignonia, Bignoniaceae, family, such as Chilopsis linearis; members of the vervain, Verbenaceae, family, including Glandularia gooddugii and Verbena neomexicana; members of the mint, Labiatae, family, such as Hyptis emoryi and Salvia columbariae; members of the broomrape, Orobanchaceae, family, such as Orobanche cooperi; members of the portulaca, Portulaceae, family, such as Talinum auriantiacum; members of the carpet-weed, Aizoaceae, family, such as Sesuvium verrucosum; members of the flax, Linaceae, family, such as Linum lewisii; members of the potato, Solanaceae, family, including Nicotiana trigonophylla and Physalis lobata; and members of the cochlospermum, Cochlospermaceae, family, such as Amoreuxia palmatifida.

Other groups of potential plants indigenous to geographical regions of interest include, but are not limited to, plants indigenous to temperate zones, plants indigenous to the Americas, and plants indigenous to North America.

In one embodiment, potential plants are selected from the group of plants set forth in Tables 6, 7, 8 and 9, i.e. the group comprising: Abelmochus esculentus; Achillea millefolium; Aconitum napellus; Acorus calamus; Actinidia arguta; Adiantum pedatum; Agastache foeniculum; Agrimonia eupatoria; Agropyron cristatum; Agropyron repens; Agrostis alba; Agrostis tofonifera; Alcea rosea; Alkanna tinctoria; Allium cepa; Allium grande; Allium porrum; Allium sativum; Allium schoenoprasum; Allium tuberosum; Althaea officinalis; Amaranthus gangeticus; Amaranthus retroflexus; Ambrosia artemisiifolia; Amelanchier sanguinea; Anthemis nobilis; Anthemis tinctorium; Apium graveolens; Arachis hypogaea; Aralia cordata; Arctium minus; Arctostaphylos uva-ursi; Armoracia rusticana; Aronia melanocarpa; Arrhenatherum elatius; Artemisia dracunculus; Asparagus officinalis; Aster sp; Atropa belladonna; Beta vulgaris; Beta vulgaris subsp. Maritima; Beta vulgaris var. condivata; Brassica napus; Brassica nigra; Brassica oleracea; Brassica rapa; Bromus inermis; Campanula rapunculus; Canna edulis; Capsella bursa-pastoris; Capsicum annuum; Capsicum frutescens; Carthamus tinctorius Carum carvi; Chelidonium majus; Chenopodium bonus—henricus; Chenopodium quinoa; Chrysanthemum leucanthemum; Chrysanthemun coronarium var. spatiosum; Chrysanthenum coronarium; Cichorium intybus; Citrullus lanatus; Cornus canadensis; Cosmos sulphureus; Crataegus sp; Crataegus submollis; Cryptotaenia canadensis; Cucumis anguria; Cucumis melo; Cucumis sativus; Cucurbita maxima; Cucurbita moschata; Cucurbita pepo; Curcuma zedoaria; Curcurbita maxima; Cymbopogon citratus; Dactylis glomerata; Datisca cannabina; Daucus arota; Dirca palustris; Dolicos lablab; Dryopteris filix-mas; Eleusine coracana; Elymus junceus; Erigeron canadensis; Eruca vesicaria; Fagopyrum esculentum; Fagopyrum tartaricum; Festuca rubra; Foeniculum vulgare; Forsythia×intermedia; Fragaria×ananassa; Galium odoratum; Gaultheria hispidula; Gentiana lutea; Glechoma hederacea; Glycine max; Glycyrrhiza glabra; Guizotia abyssinica; Hamamelis virginiana; Hedeoma pulegioides; Helianthus tuberosus; Helichrysum angustifolium; Heliotropium arborescens; Helleborus niger; Hordeum hexastichon; Hyssopus officinalis; Inula helenium; Isatis tinctoria; Lactuca serriola; Laportea canadensis; Lathyrus sativus; Lathyrus sylvestris; Laurus nobilis; Lavandula latifolia; Leonurus cardiaca; Lepidium sativum; Levisticum officinale; Linaria vulgaris; Linum usitatissimum; Lolium multiflorum; Lolium perenne; Lotus comiculatus; Lotus tetragonolobus; Lycopersicon esculentum; Malva moschata; Malva sylvestris; Malva verticillata; Matteucia pensylvanica; Medicago sativa; Melilotus albus; Melissa officinalis; Mentha piperita; Mentha pulegium; Mentha spicata; Mentha suaveolens; Momordica charantia; Nicotiana rustica; Nicotiana tabacum; Nigella sativa; Oenothera biennis; Origanum vulgare; Oryza sativa; Oxyria digyna; Pastinaca sativa; Phalaris canariensis; Phaseolus mungo; Phaseolus vulgaris; Phlox paniculata; Physalis alkekengi; Physalis ixocarpa; Physalis pruinosa; Phytolacca americana; Pimpinella anisum; Plantago coronopus; Plantago major; Poa compressa; Poa pratensis; Polygonum pensylvanicum; Polygonum persicaria; Potentilla anserina; Poterium sanquisorba; Pteridium aquilinum; Raphanus sativus; Rheum rhabarbarum; Ribes nidigrolaria; Ribes nigrum; Ribes salivum; Ribes sylvestre; Ribes uva-crispa; Ricinus communis; Rosa rugosa; Rosmarinus officinalis; Rubus allegheniensis; Rubus canadensis; Rubus idaeus; Rumex acetosella; Rumex acetosa; Rumex crispus; Rumex patientia; Rumex scutatus; Ruta graveolens; Salix purpurea; Salvia elegans; Salvia officinalis; Salvia sclarea; Satureja montana; Scuttellaria lateriflora; Secale cereale; Sesamum indicum; Setaria italica; Sium sisarum; Solanum dulcamara; Solanum melanocerasum; Solanum melongena; Solidago sp; Spinacia oleracea; Stachys affinis; Symphytum officinale; Tanacetum cinerariifolium; Tanacetum vulgare; Teucrium chamaedrys; Thymus serpyllum; Thymus vulgaris; Thymus×citriodorus; Tragopogon porrifolius; Trifolium hybridum; Trifolium pannonicum; Trifolium repens; Trigonella foenum-graecum; Triticum spelta; Triticum turgidum; Typha latifolia; Urtica dioica; Vaccinium corymbosum; Vaccinum augustifolium; Vaccinum macrocarpon; Veratrum viride; Verbascum thapsus; Viburnum trilobum; Vicia sativa; Vicia villosa; Vigna unguiculata; Vinca minor; Vitis sp.; Xanthium sibiricum; Zea mays; Ageratum conyzoides; Alchemilla mollis; Allium ampeloprasum; Amaranthus candathus; Angelica archangelica; Asclepias incarnata; Brassica cepticepa; Brassica juncea; Chichorium endivia subsp endivia; Cicer arietinum; Coix lacryma-jobi; Cynara scolymus; Cyperus esculentus; Datura metel; Datura stramonium; Dipsacus sativus; Echinochloa frumentacea; Erigeron speciosus; Errhenatherum elatius; Gaultheria procumbens; Helenium hoopesii; Helianthus animus; Helianthus strumosus; Hordeum vulgare; Humulus lupulus; Hypericum sp; Hyssopus officinalis; Iberis amara; Ipomoea batatas; Lactuca sativa; Lavandula angustifolia; Ledum groenlandicum; Lolium perenne; Malus hupehensis; Matricaria recutita; Nepeta cataria; Ocimum basilicum; Panicum miliaceum; Pennisetum alopecuroides; Petasites japonicus; Peucedanum oreaselinum; Phacelia tanacetifolia; Phalaris arundinacea; Phaseolus coccineus; Plectranthus sp.; Prunus cerasifera; Raphanus raphanistrum; Ribes grossularia; Rubus occidentalis; Ruta graveolens; Sambucus canadensis; Sambucus ebulus; Sanguisorba officinalis; Santolina chamaecyparissus; Serratula tinctoria; Silybum marianum; Solanum tuberosum; Sorghum caffrorum; Sorghum dochna; Sorghum durra; Sorghum sudanense; Tanacetum vulgare; Thymus fragantissumus; Tiarella cordifolia; Tropaeolum majus; Veronica officinalis; Vicia faba; Vigna angularia; Withania somnifera; Xanthium strumarium; Abies lasiocarpa; Agaricus bisporus; Allium ascalonicum; Amelanchier alnitolia; Ananas comosus; Anthriscus cerefolium; Aralia cordata; Aronia prunifolia; Asctinidia chinensis; Atriplex hortensis; Avena sativa; Averrhoa carambola; Betula glandulosa; Boletus edulis; Borago officinalis; Brassica Chinensis; Cantharellus ciparium; Carica papaya; Carthamus tinctorius; Castanea spp.; Chaerophyllum bulbosum; Chamaemelum nobile; Cichorium endivia; Cichorium endivia crispa; Cimicifuga racemosa; Citrullus colocynthus; Citrus limettoides; Citrus limon; Citrus paradisi; Citrus sinensis; Corchorus olitorius; Crithmum maritima; Cryptotaenia canadensis; Cucumis metuliferus; Cyclonia oblonga; Cynara scolymus; Datura stramonium; Dioscorea batatas; Diospiros kaki; Echinacea purpurea; Eriobotrya japonica; Fortunella spp; Fragaria; Ginkgo biloba; Gossypium herbaceum; Hibiscus cannabinus; Hydrastis canadensis; Hyoscyamus niger; Hypericum henryi; Hypericum perforatum; Hypomyces lactiflorum; Juniperus communis; Lentinus edodes; Linum usitatissimum; Litchi chinensis; Lonicera ramosissima; Lonicera syringantha; Lunaria annua; Malus hupehensis (Pamp.) Rehd.; Malus sp.; Mangifera indica; Manihot esculenta; Mentha arvensis; Menyanthes trifoliata; Miscanthus sinensis Andress; Monarda didyma; Monarda fistulosa; Montia perfoliata; Musa paradisiaca; Nasturtium officinale; Nephelium longana; Onobrychis viciafolia; Optunia sp.; Origanum marjonara; Panax quinquefolius L.; Passiflora spp; Persea americana; Phoenix dactylifera; Physalis sp; Pleurotus spp; Podophyllum peltatum; Polygonum aviculare Linné; Populus incrassata; Populus Tremula; Populus×petrowskyana; Prunus cerasus; Prunus persica; Prunus spp; Psidium guajaba; Psidium spp; Punica granatum; Pyrus communis; Pyrus pyrifolia; Reseda luteola; Rhamnus frangula; Rheum officinale; Rheum pahnatum; Sabal serrulata syn. Serenoa repens; Santolina; Satureja repandra; Scorzorera hipanica; Sechium edule; Setaria italica; Solidago canadensis; Solidago virgaurea; Stachys byzantina; Stipa capillata L.; Taraxacum officinale; Phaseolus acutifolius var. latifolius; Thlaspi arvense; Thymus herba-barona; Thymus pseudolanuginosus; Thymus serpyllum; Tragopogon sp.; Trichosanthes kirilowii; Trifolium incarnatum;×Triticosecale sp.; Triticum aestivum; Tsuga canadensis; Tsuga diversifolia; Tsuga F. macrophylla; Vicia faba; Vigna angularia; Weigela coracensis; Withania somnifera; Xanthium strumarium; Zingiber officinale; Achillea tomentosa; Aconitum; Allium victorialis; Amelanchier canadensis; Anthoxanthum odoratum; Arctium lappa; Asarum europaeum; Athyrium asperum; Atropa belladonna; Begonia convolvulacea; Begonia eminii; Begonia glabra; Begonia Hannii; Begonia polygonoides; Berberis vulgaris; Brassica juncea; Calendula officinalis; Camellia sinensis; Chrysanthemum balsamita; Coriandrum sativum; Filipendula rubra; Geum rivale; Hylotelephium; Iberis sempervirens; Jeffersonia diphylla; Ligularia dentata; Miscanthus sacchariflorus; Petroselium crispum; Peucedanum cervaria; Philadelphus coronarius; Physostegia virginiana; Plectranthus fruticosus; Pulmonaria saccharata; Salvia nemorosa; Saponaria officinalis; Solidago hybrida; Stellaria graminea Linné; Tamarindus indica; Thalictrum aquilegiifolium; Thuja occidentalis; Thymus praecox subsp arctitus; Yucca filamentosa; Adiantum tenerum; Anaphalis margaritacea; Angelica dahurica; Begonia manii; Betula glandulosa; Equisetum hyemale; Erysimum perofskianum Fish. S.; Foeniculum purpureum; Filipendula ulmaria; Filipendula vulgaris; Lythrum salicaire; Passiflora caerula; Pongamia pinnata; Pulmonaria officinalis; Rhus aromaticaSilene vulgaris; Tetradenia riparia; Thymus vulgaris; Argenteus; Tussilago farfara; Aesculus hippocastanum; Allium fistulosumAlpinia oficinarum; Amsonia tabemaemontana; Anaphalis margaritacea; Angelica sinensis syn. A. polymorpha; Asclepias incarnata L.; Asclepias tuberosa; Asctinidia chinensis; Crataegus oxyacanta; Butomus umbellatus; Cinnamomum sp.; Chrysanthemum parthenium; Citrus paradisi; Cocos nucifera; Crataegus sanguinea; Fucus vesiculosis; Fumaria officinalis; Gentiana macrophylla; Juglans nigra; Lochia scoparia (L.) Schrad.; Krameria Triandra; Ligustrum vulgare; Lupinus polyphyllus lindl.; Lychnis chalcedonica; Optunia sp.; Polygonium chinense; Pontederia cordata; Portulacea oleracea; Primula veris; Pulmonaria officinalis; Punica granatum; Radix Paeonia rubra; Rhus trilobata; Sambucus nigra; Sanguisorba minor; Saponaria officinalis L.; Sechium edule; Tanacetum balsamila; Aronia×prunifolia; Manihot esculenta; Angelica sinensis; Conyza canadensis, and Cynara carduculus subsp. Cardunculus.

In accordance with one embodiment of the present invention, potential plants are selected from the group of plants set forth in Tables 8 and i.e. the group comprising: Allium tuberosum; Althacea officinalis; Ambrosia artemisiifolia; Angelica sinensis; Aronia×prunifolia; Asarum europaeum; Begonia Hannii; Begonia polygonoides; Brassica napus; Brassica oleracea; Bromus inermis; Chenopodium quinoa; Citrullus lanatus; Conyza canadensis; Daucus carota; Hypomyces lactifluorum; Iberis sempervirens; Lunaria annua; Manihot esculenta; Matricaria recutita; Melilotus albus; Phaseolus vulgaris; Physostegia virginiana; Pisum sativum; Raphanus raphanistrum; Ribes sylvestre; Rubus occidentalis; Rumex crispus; Solidago canadensis; Solidago sp.; Solidago×hybrida; Tamarindus indica; Taraxacum officinale; Tropaeolum majus; Tsuga canadensis; Tsuga diversifolia; Vaccinium angustifolium; Zea mays and Zingiber officinale.

In accordance with another embodiment of the present invention, potential plants are selected from the group of plants set forth in Table 8, i.e. the group comprising: Amaranthus candathus: Ambrosia artemisiifolia; Aronia×prunifolia; Brassica napus; Brassica oleracea; Bromus inermis; Chenopodium quinoa; Citrullus lanatus; Dolichos lablab; Foeniculum vulgare; Hypomyces lactifluorum; Lotus corniculatus; Manihot esculenta; Matricaria recutita; Melilotus albus; Phaseolus vulgaris; Pisum sativum; Raphanus raphanistrum; Ribes sylvestre; Rumex crispus; Rumex scutatus; Tanacetum cinerariifolium; Tropaeolum majus; Tsuga canadensis; Tsuga diversifolia; Vaccinium angustifolium; Zea mays and Zingiber officinale.

In accordance with a further embodiment of the present invention, potential plants are selected from the group of plants set forth in Table 9, i.e. the group comprising: Allium tuberosum; Althacea officinalis; Ambrosia artemisiifolia; Angelica sinensis; Aronia×prunifolia; Asarum europaeum; Begonia Hannii; Begonia polygonoides; Brassica oleracea; Bromus inermis; Chenopodium quinoa; Conyza canadensis; Cynara cardunculus subsp. Cardunculus; Daucus carota; Hypomyces lactifluorum; Iberis sempervirens; Lunaria annua; Melilotus albus; Phaseolus vulgaris; Physostegia virginiana; Pisum sativum; Ribes sylvestre; Rubus occidentalis; Rumex crispus; Salvia officinalis; Solidago canadensis; Solidago sp.; Solidago×hybrida; Taraxacum officinale; Tsuga canadensis; Tsuga diversifolia; Zea mays and Zingiber officinale.

In accordance with a further embodiment of the present invention, the potential plant is a member of the Family Zingiberaceae, the Family Pinaceae or the Family Asteraceae. In another embodiment of the invention, the potential plant is a member of the Solidago genus, the Tsuga genus or the Zingiber genus.

In another embodiment the potential plant is selected from the group comprising: Solidago sp., Tsuga canadensis and Zingiber officinale. In a further embodiment, the potential plant is a Solidago sp. selected from the group of: Solidago canadensis, Solidago gigantea (also known as Solidago serotina), Solidago virgaurea and Solidago hybrida.

### 1.1 Preparation of Plant Extracts

Methods of preparing plant extracts have been described in detail in International Patent Application PCT/CA02/00285 (Publication No. WO 02/06992) and are suitable for use in the preparation of the plant extracts of the present invention. Other methods are known in the art and include those described herein. In accordance with one embodiment of the invention, there is provided a process for obtaining a plant extract capable of inhibiting MMP-9 and/or cathepsin B protease activity, the process comprising:
(a) obtaining plant material from one or more plants;
(b) obtaining an extract from the plant material by contacting the plant material with an aqueous, an ethanolic or an organic solvent, or a combination thereof, thereby providing one or more plant extracts;
(c) analysing the plant extract(s) for the presence of inhibitory activity against MMP-9 and/or cathepsin B proteases; and
(d) selecting plant extracts having inhibitory activity against one or both of the proteases.

Plant material can be obtained by directly harvesting the material from the selected plant(s) or it may be obtained from commercial sources.

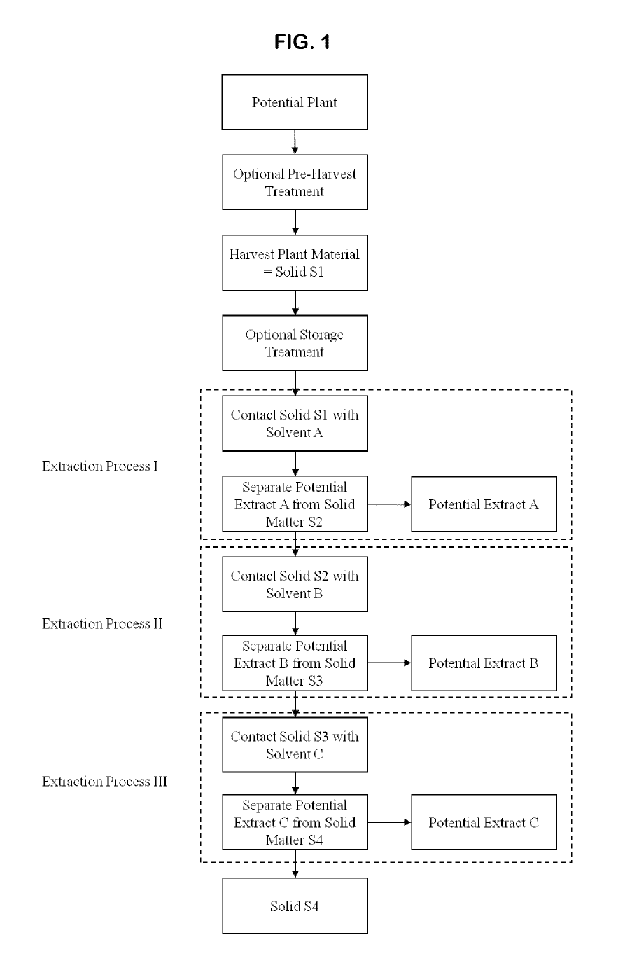

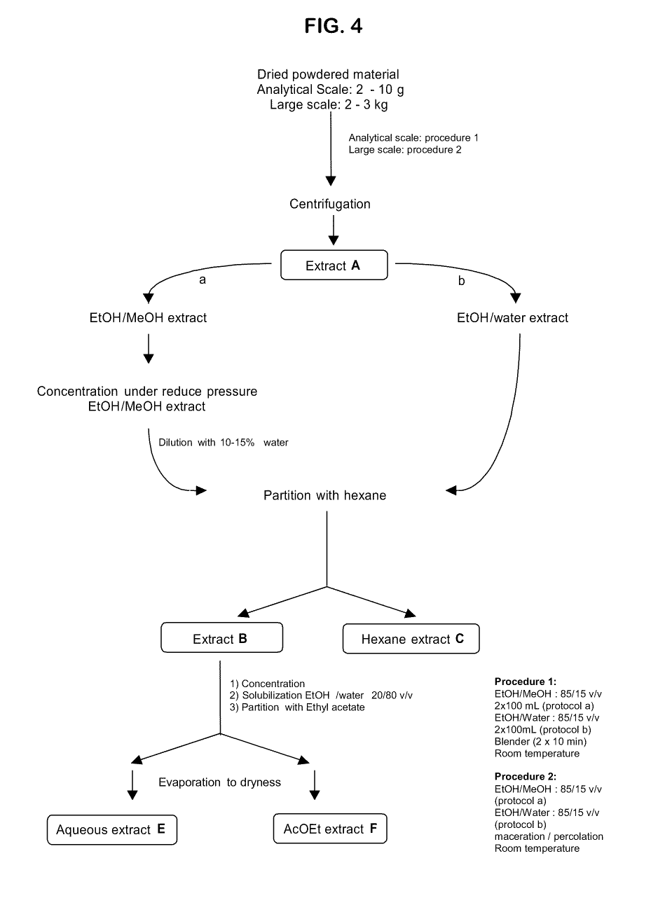

Exemplary methods of preparation are provided in FIGS. 1 and 4 and begin with the selection of a potential plant. The selected plant can optionally be subjected to a pre-harvest treatment, for example treatment with water, or treatment with water and/or a stressor or a combination of stressors. The plant can be treated for storage and stored prior to extraction or it can be used directly. Plant material from the selected plant is next treated with a solvent after which the liquid is separated from the solid material, wherein the liquid becomes Potential Extract A. The solid S2 can be further treated with a second solvent and subsequent solvents if desired to generate additional potential extracts.

### 1.1.1 Plant Stressors

As noted above, if desired, potential plants may be subjected to a pre-harvest treatment, wherein the treatment can be water or water and/or one or more stressor, elicitor, or inducer, prior to preparation of the extract. A pre-harvest treatment comprises contacting or treating a potential plant, or material from a potential plant, with water and/or one or more stressor, elicitor, or inducer. Examples of stressors, elicitors and inducers include, but are not limited to, chemical compounds, for example organic and inorganic acids, fatty acids, glycerides, phospholipids, glycolipids, organic solvents, amino acids and peptides, monosaccharides, oligosaccharides, polysaccharides and lipopolysaccharides, phenolics, alkaloids, terpenes and terpenoids, antibiotics, detergents, polyamines, peroxides, ionophores, and the like; subjection of the plant material to a physical treatment, such as ultraviolet radiation, sandblasting, low and high temperature stress, osmotic stress induced by salt or sugars, nutritional stress defined as depriving the plant of essential nutrients (e.g. nitrogen, phosphorus or potassium), in order to induce or elicit increased production of one or more chemicals. The one or more stressor (i.e. chemical compound or physical treatment) may be applied continuously or intermittently to the plant or plant material, or the potential plant can be subjected to a variety of pre-harvest treatments and an extract prepared after each treatment. Various stressors and procedures for stressing plants prior to extract preparation have been described previously (see International Patent Application WO 02/06992) and are suitable for use in the present invention.

In one embodiment of the present invention, the potential plant is treated with one or more chemical stressors. In another embodiment, the potential plant is treated with one or more stressors selected from the group of: γ-linolenic acid, γ-linolenic acid lower alkyl esters, arachidonic acid and arachidonic acid lower alkyl esters. In another embodiment, the potential plant is treated with γ-linolenic acid or arachidonic acid. In a further embodiment, the plants are subjected to a physical stress, such as sandblasting. In yet another embodiment, unstressed plants are used.

Various combinations of stressors and treatment regimes can also be employed to induce or enhance the production of one or more extracellular protease inhibitors in the plant material. One skilled in the art would be able to determine from the results of assays, such as those described herein, conducted to determine the activity of stressed and unstressed plant extracts against MMP-9 or cathepsin B whether it is desirable to follow one or more than one of the stressor regimes.

### 1.1.2 Harvesting the Plant Material for Extraction and Optional Storage Treatment

Plant material harvested from the potential plant(s) for use in the extraction procedure(s) can comprise the entire plant, or it can be one or more distinct tissues from the plant, for example, leaves, seeds, roots, stems, flowers, or various combinations thereof. The plant material may be used directly as harvested from the plant, immediately after the optional pre-harvest treatment, or it may be desirable to store the plant material for a period of time prior to performing the extraction procedure(s). If desired, the plant material can be treated prior to storage, for example, by drying, freezing, lyophilising, or some combination thereof.

Following treatment to prepare the plant material for storage, the plant material may be stored for a period of time prior to being submitted to the extraction procedure(s). The storage time may be of variable duration, for example, the storage period may be between a few days and a few years. In one embodiment of the invention, the plant material is stored for a period of less than one week. In another embodiment, the plant material is stored for a period between one week to one month. In a further embodiment, the plant material is stored for a period of between one month to six months. In other embodiments, the plant material is stored for periods of between four months to one year and for a period over one year in duration.

### 1.1.3 The Extraction Process

Various extraction processes are known in the art and can be employed in the methods of the present invention (see, for example, International Patent Application WO 02/06992). The extract is generally produced by contacting the solid plant material with a solvent with adequate mixing and for a period of time sufficient to ensure adequate exposure of the solid plant material to the solvent such that inhibitory activity present in the plant material can be taken up by the solvent.

In one embodiment of the present invention the plant material is subjected to an extraction process as depicted in FIG. 1. In accordance with this embodiment, three basic extraction processes are performed in sequence to generate potential extracts A, B and C.

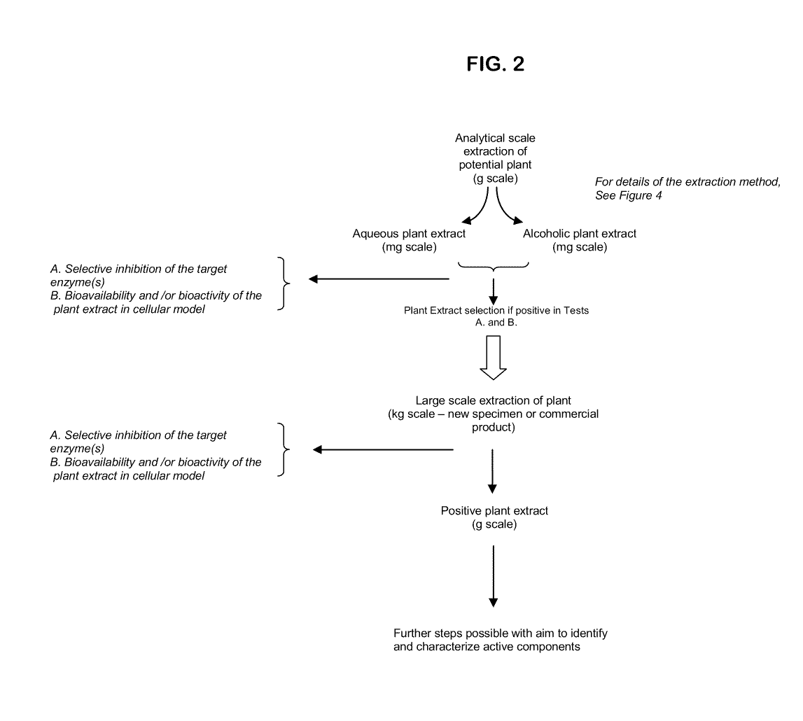

In other embodiments of the present invention, greater or fewer extraction processes are contemplated. For example, in an alternative embodiment, the plant material is subjected to an extraction process as depicted in FIG. 2. In accordance with this embodiment, the plant material is subjected to two separate extraction processes concurrently resulting in two separate potential extract A′s.

Regardless of the number of extraction processes, each extraction process typically is conducted over a period of time between about 10 minutes and about 24 hours at a temperature between about 4° C. and about 50° C. Adequate contact of the solvent with the plant material can be encouraged by shaking the suspension. The liquid fraction is then separated from the solid (insoluble) matter resulting in the generation of two fractions: a liquid fraction, which is a potential extract, and a solid fraction. Separation of the liquid and solid fractions can be achieved by one or more standard processes known to those skilled in the art.

In accordance with the embodiment depicted in FIG. 1, the extraction process is then repeated with a second and a third solvent. Solvents A, B and C in FIG. 1 generally represent separate classes of solvents, for example, aqueous, alcoholic and organic. The solvents can be applied in specific order, for example, a polar to non-polar order or in a non-polar to polar order. Alternatively, the solvents can be applied in a random sequence. In all cases, however, the solid matter should be dried prior to contact with the subsequent solvent.

The plant material employed in the extraction process can be the entire potential plant, or it can be one or more distinct tissues from the plant, for example, leaves, seeds, roots, stems, flowers, or various combinations thereof. The plant material can be fresh, dried or frozen. If desired, the plant material can be treated prior to the extraction process in order to facilitate the extraction of the inhibitory activity.

Typically such treatment results in the plant material being fragmented by some means such that a greater surface area is presented to the solvent. For example, the plant material can be crushed or sliced mechanically, using a grinder or other device to fragment the plant parts into small pieces or particles, or the plant material can be frozen liquid nitrogen and then crushed or fragmented into smaller pieces.

The solvent used for each extraction process can be aqueous, alcoholic or organic, or a combination thereof. In one embodiment of the present invention, plant material is extracted with an aqueous solvent. Examples of suitable aqueous solvents include, but are not limited to, water, buffers, cell media, dilute acids or bases and the like. Various buffers are known in the art and can be utilised as extractants in the context of the present invention. Examples include, but are not limited to, TRIS, BIS-TRIS, HEPES, PIPES, MES, BICINE, TRICINE, and CAPS. Examples of suitable cell media include, but are not limited to, 10% serum DMEM, serumless DMEM, RPMI 1640, HAM's F12, CMRL 1066, McCoy's 5A, Medium 199, Waymouth's MB752, Eagle's or Joklile's MEM, α-MEM. In another embodiment, an aqueous solvent comprising an aqueous TRIS-HCl buffer at pH 6-8 for a period of between 30 minutes to 8 hours at a temperature between about 4° C. to about 50° C. is used for the extraction.

In an alternate embodiment of the invention, plant material is extracted with an alcoholic solvent. Examples of suitable alcoholic solvents include, but are not limited to, methanol, ethanol, n-propanol, iso-propanol, n-butanol, 2-butanol, tert-butanol, and combinations thereof. In one embodiment, a combination of ethanol and methanol is used as the alcoholic solvent, wherein the range of ethanol:methanol is between about 50:50 and about 85:15. In a further embodiment, the plant material is contacted with an alcoholic solvent for a time period between about 10 minutes to one hour at a temperature between about 4° C. to about 25° C.

In an alternate embodiment, plant material is extracted with an organic solvent. Examples of suitable organic solvents include, but are not limited to, diethylether, hexane, heptane, dichloromethane, ethyl acetate, butyl alcohol, dimethylsulfoxide (DMSO), chloroform, ether, acetone, and combinations thereof. In one embodiment, dichloromethane is used as the solvent and the plant material is shaken for one to twenty-four hours with the solvent.

In an alternate embodiment, plant material is extracted with an alcoholic solvent in combination with a co-solvent, which may be aqueous or organic. In one embodiment, a combination of ethanol and water is used as the solvent, wherein the range of ethanol:water is between about 50:50 and about 85:15.

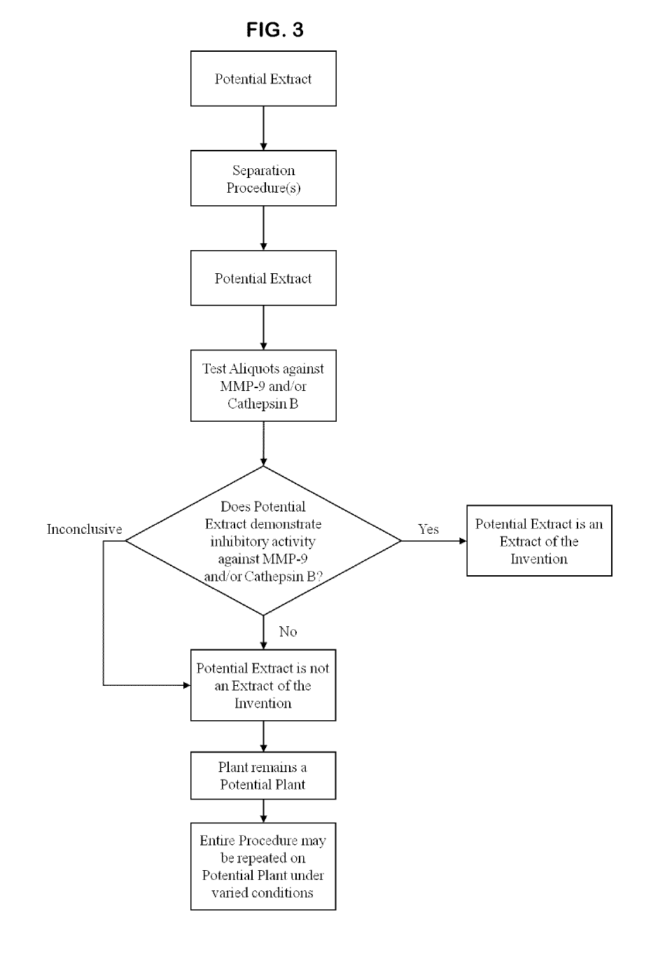

Once the potential extracts have been isolated, they can be tested directly (after being dissolved or dispersed in a suitable solvent) for their ability to inhibit extracellular protease activity, or they may be subjected to further procedures as described below and outlined in FIGS. 2 and 3. For example, the potential extracts can be subjected to procedures to remove fatty acids or chlorophyll components that may interfere with the protease activity or other assays. Various procedures known in the art may be employed. In one embodiment, one or more additional partitioning step using an organic solvent, such as hexane, heptane or ethyl acetate, is included. The liquid potential extract can be concentrated and solubilised in an appropriate solvent prior to the one or more partitioning step, if desired.

The present invention contemplates that the extraction process may be carried out on various scales including known large, medium and small-scale methods of preparing extracts.

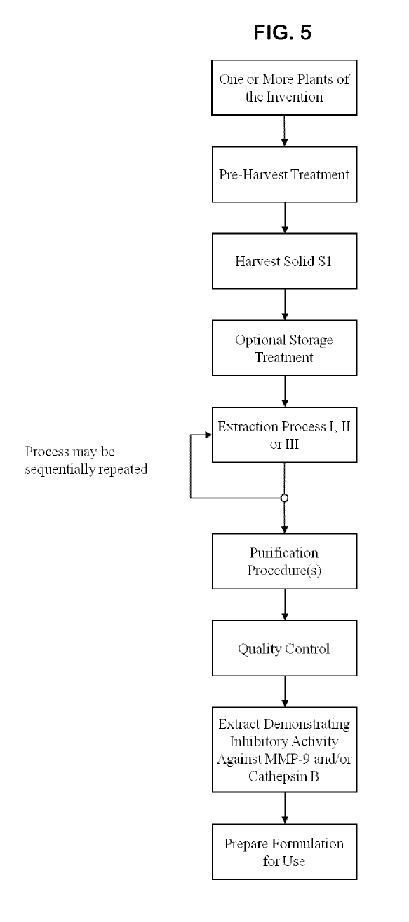

The present invention contemplates the large-scale preparation of selected plant extracts of the invention. Such extracts can be prepared on a commercial scale by repeating the extraction process that lead to the isolation of the extract of interest. One embodiment of this aspect of the invention is presented in FIG. 5. In this embodiment, the small-scale extraction procedure is simply scaled-up and additional steps of quality control are included to ensure reproducible results for the resulting extracts. Similarly the process outlined in FIG. 4 can be scaled up for commercial purposes, as indicated in FIG. 2.

Also contemplated by the present invention are modifications to the small-scale procedure that may be required during scale-up for industrial level production of the extract. Such modifications include, for example, alterations to the solvent being used or to the extraction procedure employed in order to compensate for variations that occur during scale-up and render the overall procedure more amenable to industrial scale production, or more cost effective. Modifications of this type are standard in the industry and would be readily apparent to those skilled in the art.

### 1.1.4 Purification/Fractionation of Extracts

The plant extracts of the present invention can be further purified or concentrated if desired. By “purified” it is meant that the extract has been subjected to additional purification, partial purification, and/or fractionation steps.

Such purification, partial purification, and/or fractionation can be performed using a variety of techniques known in the art including, for example, solid-liquid extraction, liquid-liquid extraction, solid-phase extraction (SPE), membrane filtration, ultrafiltration, dialysis, electrophoresis, solvent concentration, centrifugation, ultracentrifugation, liquid or gas phase chromatography (including size exclusion, affinity, etc.) with or without high pressure, lyophilisation, evaporation, precipitation with various “carriers” (including PVPP, carbon, antibodies, etc.), or various combinations thereof. One skilled in the art, would appreciate how to use such options, in a sequential fashion, in order to enrich each successive fraction in the activity of interest (i.e. inhibition of MMP-9 and/or cathepsin B) by following the activity throughout the purification procedure.

Solid-liquid extraction means include the use of various solvents in the art, and includes the use of supercritical solvents, soxhlet extractors, vortex shakers, ultrasounds and other means to enhance extraction, as well as recovery by filtration, centrifugation and related methods as described in the literature (see, for example, R. J. P. Cannell, Natural Products Isolation, Humana Press, 1998). Examples of solvents that may be used include, but are not limited to, hydrocarbon solvents, chlorinated solvents, organic esters, organic ethers, alcohols, water, and mixtures thereof. In the case of supercritical fluid extraction, the invention also covers the use of modifiers such as those described in V. H. Bright (Supercritical Fluid Technology, ACS Symp. Ser. Vol. 488, ch. 22, 1999).

Liquid-liquid extraction means include the use of various mixtures of solvents known in the art, including solvents under supercritical conditions. Typical solvents include, but are not limited to, hydrocarbon solvents, chlorinated solvents, organic esters, organic ethers, alcohols, water, various aqueous solutions, and mixtures thereof. The liquid-liquid extraction can be effected manually, or it can be semi-automated or completely automated, and the solvent can be removed or concentrated by standard techniques in the art (see, for example, S. Ahuja, Handbook of Bioseparation, Academic Press, 2000).

Solid-phase extraction (SPE) techniques include the use of cartridges, columns or other devices known in the art. The sorbents that may be used with such techniques include, but are not limited to, silica gel (normal phase), reverse-phase silica gel (modified silica gel), ion-exchange resins, and fluorisil. The invention also includes the use of scavenger resins or other trapping reagents attached to solid supports derived from organic or inorganic macromolecular materials.

Membrane, reverse osmosis and ultrafiltration means include the use of various types of membranes known in the art, as well as the use of pressure, vacuum, centrifugal force, and/or other means that can be utilised in membrane and ultrafiltration processes (see, for example, S. Ahuja, Handbook of Bioseparations, Academic Press, 2000).

Dialysis means include membranes having a molecular weight cut-off varying from less than about 0.5 KDa to greater than about 50 KDa. The invention also covers the recovery of purified and/or fractionated extracts from either the dialysate or the retentate by various means known in the art including, but not limited to, evaporation, reduced pressure evaporation, distillation, vacuum distillation, and lyophilization.

Chromatographic means include various means of carrying out chromatography known by those skilled in the art and described in the literature (see, for example, G. Sofer, L. Hagel, Handbook of Process Chromatography, Academic Press, 1997). Examples include, but are not limited to, regular column chromatography, flash chromatography, high performance liquid chromatography (HPLC), medium pressure liquid chromatography (MPLC), supercritical fluid chromatography (SFC), countercurrent chromatography (CCC), moving bed chromatography, simulated moving bed chromatography, expanded bed chromatography, and planar chromatography. With each chromatographic method, examples of sorbents that may be used include, but are not limited to, silica gel, alumina, fluorisil, cellulose and modified cellulose, various modified silica gels, ion-exchange resins, size exclusion gels and other sorbents known in the art (see, for example, T. Hanai, HPLC: A Practical Guide, RSC Press, UK 1999). The present invention also includes the use of two or more solvent gradients to effect the fractionation, partial purification, and/or purification steps by chromatographic methods. Examples of solvents that may be utilised include, but are not limited to, hexanes, heptane, pentane, petroleum ethers, cyclohexane, heptane, diethyl ether, methanol, ethanol, isopropanol, propanol, butanol, isobutanol, tert-butanol, water, dichloromethane, dichloroethane, ethyl acetate, tetrahydrofuran, dioxane, tert-butyl methyl ether, acetone, and 2-butanone. When water or an aqueous phase is used, it may contain varying amounts of inorganic or organic salts, and/or the pH may be adjusted to different values with an acid or a base such that fractionation and/or purification is enhanced.

In the case of planar chromatography, the present invention includes the use of various forms of this type of chromatography including, but not limited to, one- and two dimension thin-layer chromatography (1D- and 2D-TLC), high performance thin-layer chromatography (HPTLC), and centrifugal thin-layer chromatography (centrifugal TLC).

In the case of countercurrent chromatography (CCC), the present invention includes the use of manual, semi-automated, and automated systems, and the use of various solvents and solvent combinations necessary to effect the fractionation and/or purification steps (see, for example, W. D. Conway, R. J. Petroski, Modern Countercurrent Chromatography, ACS Symp. Ser. Vol. 593, 1995). Solvent removal and/or concentration can be effected by various means known in the art including, but not limited to, reduced pressure evaporation, evaporation, reduced pressure distillation, distillation, and lyophilization.

The present invention includes fractionation, partial purification, and purification by expanded bed chromatography, moving and simulated moving bed chromatography, and other related methods known in the art (see, for example, G. Sofer, L. Hagel, Handbook of Process Chromatography, Academic Press, 1997 and S. Ahuja, Handbook of Bioseparations, Academic Press, 2000).

Selective precipitation means includes the use of various solvents and solvent combinations, the use of temperature changes, the addition of precipitant and/or modifiers, and/or modification of the pH by addition of base or acid to effect a selective precipitation.

The invention also includes fractionation, partial purification, and/or purification by steam distillation, hydrodistillation, or other related methods of distillation known in the art (see, for example, L. M. Harwood, C. J. Moody, Experimental Organic Chemistry, Blackwell Scientific Publications, UK, 1989).

The process of purifying also includes the concentration of purified or partially purified extracts by solvent removal from the original extract and/or fractionated extract, and/or purified extract. The techniques of solvent removal are known to those skilled in the art and include, but are not limited to, rotary evaporation, distillation (normal and reduced pressure), centrifugal vacuum evaporation (speed-vac), and lyophilization.

1.2 Determination of the Ability of the Plant Extracts to Inhibit MMP-9 and/or Cathepsin B Activity

As indicated above, potential plant extracts for inclusion in the therapeutic compositions of the invention are capable of inhibiting the activity of MMP-9 and/or cathepsin B. Potential extracts can be tested for their ability to inhibit these proteases using a variety of techniques known in the art, including, but not limited to, those described herein. In the context of the present invention, a plant extract that decreases the activity of MMP-9 and/or cathepsin B by at least 20% is considered to be capable of inhibiting the activity of that protease. Thus, in accordance with one embodiment of the invention there is provided a method of screening for plant extracts suitable for inclusion in the therapeutic compositions, the method comprising:
(a) providing one or more plant extracts;
(b) analysing the one or more extracts for inhibitory activity against MMP-9 and/or cathepsin B; and
(c) selecting extracts that decrease the activity of MMP-9 and/or cathepsin B by at least 20%, as plant extracts suitable for inclusion in the therapeutic compositions.

Potential extracts can be tested directly against MMP-9 and/or cathepsin B or they may have been submitted to a preliminary screen, for example, against a panel of known extracellular proteases (EPs) with those extracts that are capable of inhibiting at least one EP being selected for further testing. EPs that may be used in such a preliminary screening step include, but are not limited to, matrix metalloproteinases (MMPs), cathepsins, elastase, plasmin, TPA, uPA, kallikrein, ADAMS family members, neprilysin, gingipain, clostripain, thermolysin, serralysin, and other bacterial and viral proteases.

One skilled in the art would appreciate that there are a variety of methods and techniques for measuring qualitatively and/or quantitatively the ability of a plant extract to inhibit the activity of MMP-9 and/or cathepsin B.

For example, there are currently several assays to measure the activity of various MMPs, including MMP-9, elastases and cathepsins (for a review of these methods, see Murphy and Crabbe, In Barrett (ed.) Methods in Enzymology. Proteolytic Enzymes: Aspartic Acid and Metallopeptidases, New York: Academic Press, 1995, 248: 470), including the gelatinolytic assay (which is based on the degradation of radio-labelled type I collagen), the zymography assay (which is based on the presence of negatively-stained bands following electrophoresis through substrate-impregnated SDS polyacrylamide gels) and a microtitre plate assay developed by Pacmen et al., (Biochem. Pharm. (1996) 52:105-111).

Other methods include those that employ auto-quenched fluorogenic substrates. Many fluorogenic substrates have been designed for quantification of the activity of MMPs, elastase, and cathepsins through fluorescent level variation measuring (reviewed by Nagase and Fields (1996) Biopolymers 40: 399-416). For example, the auto-quenched fluorogenic peptide substrate MCA-Pro-Leu-Gly-Leu-Dpa-Ala-Arg-NH₂ can be used for assaying the activity of MMP-9 and is commercially available from Calbiochem (San Diego, Calif., USA). The auto-quenched peptide substrate Z-Arg-Arg-AMC, also commercially available from Calbiochem, is suitable for the assessment of cathepsin B activity. Cathepsin B activity can also be assayed using haemoglobin that is heavily labelled with Alexa-488 dye (Molecular Probes, Eugene, Oreg.).

Fluorescence polarization assays are based on the principle that when fluorescent molecules are excited with plane polarized light, they will emit light in the same polarized plane provided that the molecule remains stationary throughout the excited state. However, if the excited molecule rotates or tumbles during the excited state, then light is emitted in a plane different from the excitation plane. If vertically polarized light is used to excite the fluorophore, the emission light intensity can be monitored in both the original vertical plane and also the horizontal plane. The degree to which the emission intensity moves from the vertical to horizontal plane is related to the mobility of the fluorescently labelled molecule. If fluorescently labelled molecules are very large, they move very little during the excited state interval, and the emitted light remains highly polarized with respect to the excitation plane. If fluorescently labelled molecules are small, they rotate or tumble faster, and the resulting emitted light is depolarized relative to the excitation plane. Therefore, FP can be used to follow any biochemical reaction that results in a change in molecular size of a fluorescently labelled molecule (e.g. protein-DNA interactions; immunoassays; receptor-ligand interactions; degradation reactions). (Adapted from Bolger R, Checovich W. (1994) Biotechniques 17(3):585-9.).

Another method of measuring extracellular protease activity makes use of the fluorescent activated substrate conversion (FASC) assay described in Canadian Patent No. 2,189,486 (1996) and in St-Pierre et al., (1996) Cytometry 25: 374-380.

Various formats known in the art may be employed if the potential extracts are to be tested against a panel of EPs, or if a plurality of extracts are to be tested against a single EP, such as MMP-9 or cathepsin B, or both MMP-9 and cathepsin B simultaneously. For example, the potential extracts may be tested against one or more protease in a sequential fashion or against a plurality of proteases, such as an array of extracellular proteases, simultaneously, or a plurality of plant extracts can be tested simultaneously against one or more EPs. The assays may be adapted to high throughput in order to facilitate the simultaneous testing of potential extracts. High throughput techniques are constantly being developed and the use of such techniques to adapt the assays in the future is also considered to be within the scope of the present invention.

In accordance with one embodiment of the present invention, plant extracts that are capable of selectively inhibiting MMP-9 or cathepsin B are selected. By “selectively inhibiting” it is meant that the extract inhibits MMP-9 or cathepsin B to a greater extent than other EPs. Selective inhibition can be determined by measurement of IC₅₀ values as is known in the art. An IC₅₀ is defined as the concentration of extract at which 50% inhibition of protease catalytic activity occurs. In accordance with the present invention, a plant extract is considered to selectively inhibit MMP-9 or cathepsin B when it inhibits the selected protease with an IC₅₀ value at least one half log lower than the IC₅₀ value against other EPs. In order to determine whether an extract is capable of selectively inhibiting MMP-9 and/or cathepsin B, the extract should be tested against MMP-9 and/or cathepsin B and at least one other EP using methods such as those described above and the IC₅₀ values determined. If, on comparison of the IC₅₀ values, the IC₅₀ value for the extract against MMP-9/cathepsin B is at least one half log lower than the IC₅₀ value for the extract against the at least one other EP, then the extract is considered to selectively inhibit MMP-9/cathepsin B.

### 2. Synthetic MMP-9 and Cathepsin B Inhibitors

As indicated above, the therapeutic compositions of the present invention can further comprise one or more synthetic MMP-9 and/or cathepsin B inhibitor. As these phyto-synthetic compositions simultaneously target MMP-9 and cathepsin B, they are also useful in the treatment of cancer. A number of synthetic compounds capable of inhibiting MMP-9 or cathepsin B are known in the art and can be included in the compositions of the invention. Examples include, but are not limited to, marimastat, prinomastat, tanomastat, metastat, E-64, CA-074 methyl-ester, leupeptin, 1-phenyl-1,4-epoxy-1H,4H-naphtho[1,8-de][1,2]dioxepin (ANO-2) and ilomastat (also known as N-[(2R)-2-(hydroxamidocarbonylmethyl)-4-methylpentanoyl]-L-tryptophan methylamide, Galardin™ or GM-6001). It will be understood that other synthetic inhibitors may be developed in the future that will also be suitable for use in the compositions of the present invention.

### Anti-Cancer Therapeutics

As indicated above, the present invention contemplates therapeutic combinations comprising a therapeutic composition in combination with one or more anti-cancer therapeutics. In the context of the present invention, “anti-cancer therapeutics” include a wide variety of compounds, compositions and treatments that prevent or delay the growth and/or metastasis of cancer cells. Such anti-cancer therapeutics include, for example, chemotherapeutic drugs, radiation therapy, gene therapy, hormonal manipulation, immunotherapeutics, alternative therapy (including the use of naturopathic preparations), and antisense oligonucleotide therapy.

In one embodiment of the present invention, the compositions are used in combination with one or more chemotherapeutic drugs, one or more immunotherapeutics, or one or more naturopathic preparations.

### 1. Chemotherapeutics

Suitable chemotherapeutics for use in combination with the therapeutic compositions of the invention can be selected from a wide range of cancer chemotherapeutic agents known in the art. Known chemotherapeutic agents include those that are applicable to the treatment of a range of cancers (i.e. broad-spectrum chemotherapeutics), such as doxorubicin, capecitabine, mitoxantrone, irinotecan (CPT-11), cisplatin and gemcitabine, as well as those that are specific for the treatment of a particular type of cancer.

For example, etoposide is generally applicable in the treatment of leukaemias (including acute lymphocytic leukaemia and acute myeloid leukaemia), germ cell tumours, Hodgkin's disease and various sarcomas. Cytarabine (Ara-C) is also applicable in the treatment of various leukaemias, including acute myeloid leukaemia, meningeal leukaemia, acute lymphocytic leukaemia, chronic myeloid leukaemia, erythroleukaemia, as well as non-Hodgkin's lymphoma.

The present invention contemplates the use of both types of chemotherapeutic agent in combinations with the therapeutic compositions of the invention. In one embodiment of the invention, the therapeutic compositions are used in combination with one or more broad spectrum chemotherapeutic. In another embodiment of the invention, the therapeutic combination comprises cisplatin or doxorubicin.

Exemplary chemotherapeutics that can be used alone or in various combinations for the treatment specific cancers are provided in Table 1. One skilled in the art will appreciate that many other chemotherapeutics are available and that the following list is representative only.

TABLE 1
Exemplary Chemotherapeutics Used in the Treatment of Some
Common Cancers

| CANCER | CHEMOTHERAPEUTIC | |
|---|---|---|
| Acute lymphocytic | Pegaspargase (e.g. Oncaspar ®) | L-asparaginase |
| leukaemia (ALL) | Cytarabine | |
| Acute myeloid | Cytarabine | Idarubicin |
| leukaemia (AML) | | |
| Brain cancer | Procarbazine (e.g. Matulane ®) | Nitrosoureas |
| | Platinum analogues | Temozolomide |
| Breast cancer | Capecitabine (e.g. Xeloda ®) | Cyclophosphamide |
| | 5-fluorouracil (5-FU) | Carboplatin |
| | Paclitaxel (e.g. Taxol ®) | Cisplatin |
| | Docetaxel (e.g. Taxotere ®) | Ifosfamide |
| | Epi-doxorubicin (epirubicin) | Doxorubicin (e.g. Adriamycin ®) |
| | Tamoxifen | |
| Chronic myeloid | Cytarabine | |
| leukaemia (CML) | | |
| Colon cancer | Edatrexate (10-ethyl-10-deaza-aminopterin) | |
| | Methyl-chloroethyl-cyclohexyl-nitrosourea | |
| | 5-fluorouracil (5-FU) | Oxaliplatin |
| | Fluorodeoxyuridine (FUdR) | Vincristine |
| | Capecitabine (e.g. Xeloda ®) | |
| Colorectal cancer | Irinotecan (CPT-11, e.g. Camptosar ®) | |
| | Loperamide (e.g. Imodium ®) | Levamisole |
| | Topotecan (e.g. Hycamtin ®) | Methotrexate |
| | Capecitabine (e.g. Xeloda ®) | Oxaliplatin |
| | 5-fluorouracil (5-FU) | |
| Gall bladder | 5-fluorouracil (5-FU) | |
| Genitourinary cancer | Docetaxel (e.g. Taxotere ®) | |
| Head and neck | Docetaxel (e.g. Taxotere ®) | Cisplatin |
| cancer | | |
| Non-Hodgkin's | Procarbazine (e.g. Matulane ®) | Cytarabine |
| Lymphoma | Etoposide | |
| Non-small-cell lung | Vinorelbine Tartrate (e.g. Navelbine ®) | |
| (NSCL) cancer | Irinotecan (CPT-11, e.g. Camptosar ®) | |
| | Docetaxel (e.g. Taxotere ®) | Paclitaxel (e.g. Taxol ®) |
| | Gemcitabine (e.g. Gemzar ®) | Topotecan |
| Oesophageal cancer | Porfimer Sodium (e.g. Photofrin ®) | |
| | Cisplatin | |
| Ovarian cancer | Irinotecan (CPT-11, e.g. Camptosar ®) | |
| | Topotecan (e.g. Hycamtin ®) | |
| | Docetaxel (e.g. Taxotere ®) | Paclitaxel (e.g. Taxol ®) |
| | Gemcitabine (e.g. Gemzar ®) | Amifostine (e.g. Ethyol ®) |
| Pancreatic cancer | Irinotecan (CPT-11, e.g. Camptosar ®) | |
| | Gemcitabine (e.g. Gemzar ®) | 5-fluorouracil (5-FU) |
| Promyelocytic | Tretinoin (e.g. Vesanoid ®) | |
| leukaemia | | |
| Prostate cancer | Goserelin Acetate (e.g. Zoladex ®) | |
| | Mitoxantrone (e.g. Novantrone ®) | |
| | Prednisone (e.g. Deltasone ®) | Liarozole |
| | Nilutamide (e.g. Nilandron ®) | Flutamide (e.g. Eulexin ®) |
| | Finasteride (e.g. Proscar ®) | Terazosin (e.g. Hytrin ®) |
| | Doxazosin (e.g. Cardura ®) | Cyclophosphamide |
| | Docetaxel (e.g. Taxotere ®) | Estramustine |
| | Luteinizing hormone releasing hormone agonist | |
| Renal cancer | Capecitabine (e.g. Xeloda ®) | |
| | Gemcitabine (e.g. Gemzar ®) | |
| Small cell lung | Cyclophosphamide | Vincristine |
| cancer | Doxorubicin | Etoposide |
| Solid tumours | Gemicitabine (e.g. Gemzar ®) | Cyclophosphamide |
| | Capecitabine (e.g. Xeloda ®) | Ifosfamide |
| | Paclitaxel (e.g. Taxol ®) | Cisplatin |
| | Docetaxel (e.g. Taxotere ®) | Carboplatin |
| | Epi-doxorubicin (epirubicin) | Doxorubicin (e.g. Adriamycin ®) |
| | 5-fluorouracil (5-FU) | |

As indicated above, more than one chemotherapeutic may be employed in the combinations. It is well known in the art that standard cancer chemotherapeutics are frequently combined in order to treat a specific cancer and such combinations can be further combined with the therapeutic compositions of the invention.

Exemplary chemotherapeutic combination therapies include, for the treatment of breast cancers the combination of epirubicin with paclitaxel or docetaxel, or the combination of doxorubicin or epirubicin with cyclophosphamide. Polychemotherapeutic regimens are also useful and may consist, for example, of doxorubicin/cyclophosphamide/5-fluorouracil or cyclophosphamide/epirubicin/5-fluorouracil. Many of the above combinations are useful in the treatment of a variety of other solid tumours.

Combinations of etoposide with either cisplatin or carboplatin are used in the treatment of small cell lung cancer. In the treatment of stomach or oesophageal cancer, combinations of doxorubicin or epirubicin with cisplatin and 5-fluorouracil are useful. For colorectal cancer, CPT-11 in combination with 5-fluorouracil-based drugs, or oxaliplatin in combination with 5-fluorouracil-based drugs can be used. Oxaliplatin may also be used in combination with capecitabine.

Other examples include the combination of cyclophosphamide, doxorubicin, vincristine and prednisone in the treatment of non-Hodgkin's lymphoma; the combination of doxorubicin, bleomycin, vinblastine and dacarbazine (DTIC) in the treatment of Hodgkin's disease and the combination of cisplatin or carboplatin with any one, or a combination, of gemcitabine, paclitaxel, docetaxel, vinorelbine or etoposide in the treatment of non-small cell lung cancer.

Various sarcomas are treated by combination therapy, for example, for osteosarcoma combinations of doxorubicin and cisplatin or methotrexate with leucovorin are used; for advanced sarcomas etoposide can be used in combination with ifosfamide; for soft tissue sarcoma doxorubicin or dacarbazine can be used alone or, for advanced sarcomas doxorubicin can be used in combination with ifosfamide or dacarbazine, or etoposide in combination with ifosfamide.

Ewing's sarcoma/peripheral neuroectodermal tumour (PNET) or rhabdomyosarcoma can be treated using etoposide and ifosfamide, or a combination of vincristine, doxorubicin and cyclophosphamide. The alkylating agents cyclophosphamide, cisplatin and melphalan are also often used in combination therapies with other chemotherapeutics in the treatment of various cancers.

### 2. Immunotherapeutics

The present invention further contemplates the use of a therapeutic compositions of the invention in combination with one or more immunotherapeutic agents. Combinations comprising a therapeutic composition, chemotherapeutic(s) and immunotherapeutic(s) are also contemplated. As is known in the art, immunotherapeutic agents can be non-specific, i.e. boost the immune system generally so that it becomes more effective in fighting the growth and/or spread of cancer cells, or they can specific, i.e. targeted to the cancer cells themselves. Immunotherapy regimens may combine the use of non-specific and specific immunotherapeutic agents.

Non-specific immunotherapeutic agents are substances that stimulate or indirectly augment the immune system. Some of these agents can be used alone as the main therapy for the treatment of cancer. Alternatively, non-specific immunotherapeutic agents may be given in addition to a main therapy and thus function as an adjuvant to enhance the effectiveness of other therapies (e.g. cancer vaccines) or reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

Suitable cytokines for use in the combination therapies of the present invention include interferons, interleukins and colony-stimulating factors. Interferons (IFNs) include the common types of IFNs, IFN-alpha (IFN-α.), IFN-beta (IFN-β) and IFN-gamma (IFN-γ). Recombinant IFN-α is available commercially as Roferon (Roche Pharmaceuticals) and Intron A (Schering Corporation). Interleukins include IL-2 (or aldesleukin), IL-4, IL-11 and IL-12 (or oprelvekin). Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. An interleukin-immunotoxin conjugate known as denileukin diftitox (or Ontak; Seragen, Inc), which comprises IL-2 conjugated to diptheria toxin, has been approved by the FDA for the treatment of cutaneous T cell lymphoma. Colony-stimulating factors (CSFs) include granulocyte colony stimulating factor (G-CSF or filgrastim), granulocyte-macrophage colony stimulating factor (GM-CSF or sargramostim) and erythropoietin (epoetin alfa, darbepoietin). Various recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erythropoietin).

Non-cytokine adjuvants suitable for use in the combinations of the present invention include, but are not limited to, levamisole, alum hydroxide (alum), bacillus Calmette-Guerin (BCG), incomplete Freund's Adjuvant (IFA), QS-21, DETOX, Keyhole limpet hemocyanin (KLH) and dinitrophenyl (DNP).

The present invention further contemplates the use of one or more monoclonal antibodies in combination with therapeutic composition for the treatment of cancer. Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22.

### 3. Naturopathic Therapy

The present invention further contemplates the use of therapeutic compositions, for example as a nutraceutical formulation, in combination with one or more naturopathic preparations as part of a naturopathic therapy. For the purposes of the present invention, the term “naturopathic therapy” is intended to encompass various naturopathic, herbal, nutritional, botanical, homeopathic, alternative, and complementary therapies available for the treatment of cancer.

Examples of suitable naturopathic preparations include, but are not limited to, herbal preparations and teas including comfrey, ginseng, green tea, sassafras, Manchurian (or Kombucha) tea, Chaparral tea, Taheebo tea, Essaic, and Iscador; antineoplastons; vitamins; coenzymes; minerals; “Cancell;” 714-X; Hoxsey herbal tonic; hydrazine sulphate; dimethyl sulphoxide (DMSO); ozone; hydrogen peroxide; bioflavanoids, and shark cartilage.

### Efficacy of the Therapeutic Compositions

In accordance with the present invention, therapeutic compositions which are capable of simultaneously inhibiting MMP-9 and cathepsin B activity, are useful in the treatment of cancer. The therapeutic compositions of the invention are capable of inhibiting one or more of neoplastic cell migration, endothelial cell migration, tumour growth, and tumour metastasis, and the activity of the compositions can be initially determined in vitro if desired. The present invention thus contemplates a preliminary in vitro screening step to further characterise candidate plant extracts suitable for incorporation into the therapeutic compositions. A number of standard tests to determine the ability of a test compound or composition to inhibit cell migration, invasion and/or proliferation are known in the art and can be employed to test the plant extracts and therapeutic compositions. Exemplary procedures are described herein. When a composition comprises more than one plant extract, each extract may be tested in vitro and/or in vivo prior to combining the extracts to form the final composition if desired. The inhibitory ability of combinations of therapeutic compositions and one or more anti-cancer therapeutics can be tested by similar methods.

### 1. In Vitro Testing

Representative examples of methods of testing the activity of the compositions in vitro are outlined below and described in Examples V, VIII and IX.

In general, the ability of a plant extract or a therapeutic composition of the invention to inhibit migration/invasion of endothelial and/or neoplastic cells can be assessed in vitro using standard cell migration assays. Typically, such assays are conducted in multi-well plates, the wells of the plate being separated by a suitable membrane into top and bottom sections. The membrane is coated with an appropriate compound, the selection of which is dependent on the type of cell being assessed and can be readily determined by one skilled in the art. Examples include collagen or gelatine for endothelial cells and Matrigel for neoplastic cell lines. An appropriate chemoattractant, such as EGM-2, IL-8, α-FGF, β-FGF, fetal calf serum or the like, is added to the bottom chamber. An aliquot of the test cells together with the plant extract or therapeutic composition are added to the upper chamber, typically various dilutions of the plant extract/composition are tested. After a suitable incubation time, the membrane is rinsed, fixed and stained. The cells on the upper side of the membrane are wiped off, and then randomly selected fields on the bottom side are counted using standard methodology.

Inhibition of cell migration can also be assessed using a cord formation assay. For example, endothelial cells with or without the plant extract or composition are plated onto a suitable matrix, such as Matrigel™. After a suitable incubation period (for example, between 18 and 24 hours), the formation of any 3-dimensional capillary-like structures, or “cords,” is determined by visual inspection and/or image analysis.

The cytotoxicity of the extracts and compositions can be assayed in vitro using a suitable cancer cell line. In general, cells of the selected test cell line are grown to an appropriate density and the candidate compound is added. After an appropriate incubation time (for example, about 48 to 72 hours), cell survival is assessed. Methods of determining cell survival are well known in the art and include, but are not limited to, the resazurin reduction test (see Fields & Lancaster (1993) Am. Biotechnol. Lab. 11:48-50; O'Brien et al., (2000) Eur. J. Biochem. 267:5421-5426 and U.S. Pat. No. 5,501,959), the sulforhodamine assay (Rubinstein et al., (1990) J. Natl. Cancer Inst. 82:113-118) or the neutral red dye test (Kitano et al., (1991) Euro. J. Clin. Investg. 21:53-58; West et al., (1992) J. Investigative Derm. 99:95-100). Cytotoxicity is determined by comparison of cell survival in the treated culture with cell survival in one or more control cultures, for example, untreated cultures and/or cultures pre-treated with a control compound (typically a known therapeutic).

Similarly the ability of the plant extracts and compositions to inhibit cell proliferation can be assessed in vitro using standard techniques. Typically cells from a cell line of interest, such as a cancer or endothelial cell line, in a suitable medium. After an appropriate incubation time, the cells can be treated with the plant extract/composition and incubated for a further period of time. Cells are then counted and compared to an appropriate control. Suitable controls include, for example, cells treated with a standard therapeutic and/or untreated cells. Alternatively, the effect of the extract/composition on cell proliferation can be determined using a ³H-thymidine uptake assay. The MTT Cell Proliferation Assay can also be used to determine the effect of the plant extracts/compositions on cell proliferation rate and/or cell viability. Yellow tetrazolium MIT (3-(4,5-dimethylthiazolyl-2)-2,5-diphenyltetrazolium bromide) is reduced by metabolically active cells to generate formazan, which can be solubilized and quantified by spectrophotometric means.

Various cell lines can be used in the above assays. Examples of suitable endothelial cell lines include, but are not limited to, human umbilical vein endothelial cells (HUVECs), bovine aortic endothelial cells (BAECs), human coronary artery endothelial cells (HCAECs), bovine adrenal gland capillary endothelial cells (BCE), bovine choroidal endothelial cells and vascular smooth muscle cells. HUVECs can be isolated from umbilical cords using standard methods (see, for example, Jaffe et al. (1973) J. Clin. Invest. 52: 2745), or they can be obtained from the ATCC or various commercial sources, as can other suitable endothelial cell lines. Suitable neoplastic cell lines are available from the American Type Culture Collection (ATCC), which currently provides 950 cancer cell lines, and other commercial sources.

One skilled in the art will appreciate that it may be desirable to determine the ability of the compositions to inhibit cell migration of certain specific cancer cell lines, for example drug-resistant or highly metastatic cell lines and that appropriate cell lines can be selected accordingly.

### 2. In Vivo Testing

The ability of the therapeutic compositions of the invention to inhibit cell migration, tumour growth and/or tumour metastasis in vivo can be assessed using various standard techniques. For example, the ability of the therapeutic compositions to inhibit endothelial cell migration can be determined using the chick chorioallantoic membrane (CAM) assay, Matrigel plug assay and/or corneal micropocket assay, and the ability of the compositions to inhibit neoplastic cell migration can be assessed using various murine models of tumour growth and metastasis.

The CAM assay is a standard assay that is used to evaluate the ability of a test compound to inhibit the growth of blood vessels into various tissues, i.e. both angiogenesis and neovascularization (see Brooks et al., in Methods in Molecular Biology, Vol. 129, pp. 257-269 (2000), ed. A. R. Howlett, Humana Press Inc., Totowa, N.J.; Ausprunk et al, (1975) Am. J. Pathol., 79:597-618; Ossonski et al., (1980) Cancer Res., 40:2300-2309). Since the CAM assay measures neovascularization of whole tissue, wherein chick embryo blood vessels grow into the chorioallantoic membrane (CAM) or into the tissue transplanted on the CAM, it is a well-recognised assay model for in vivo angiogenesis.

The Matrigel™ plug assay is also a standard method for evaluating the anti-angiogenic properties of compounds in vivo (see, for example, Passaniti, et al., (1992) Lab. Invest. 67:519-528). In this assay, a test compound is introduced into cold liquid Matrigel which, after subcutaneous injection into a suitable animal model, solidifies and permits penetration by host cells and the formation of new blood vessels. After a suitable period of time, the animal is sacrificed and the Matrigel plug is recovered, usually together with the adjacent subcutaneous tissues. Assessment of angiogenesis in the Matrigel plug is achieved either by measuring haemoglobin or by scoring selected regions of histological sections for vascular density, for example by immunohistochemistry techniques identifying specific factors such as hemagglutinin (HA), CD31 (platelet endothelial cell adhesion molecule-1) or Factor VIII. Modifications of this assay have also been described (see, for example, Akhtar et al., (2002) Angiogenesis 5:75-80; Kragh et al., (2003) Int J Oncot 22:305-11).

The corneal micropocket assay is usually conducted in mice, rats or rabbits and has been described in detail by others (see D'Amato, et al., (1994)Proc. Nad Acad. Sci. USA, 91:4082-4085; Koch et al., (1991) Agents Actions, 34:350-7; Kenyon, et al., (1996) Invest. Opthalmol. Vis. Sci. 37:1625-1632). Briefly, pellets for implantation are prepared from sterile hydron polymer containing a suitable amount of the test compound. The pellets are surgically implanted into corneal stromal micropockets created at an appropriate distance medial to the lateral corneal limbus of the animal. Angiogenesis can be quantitated at various times after pellet implantation through the use of stereomicroscopy. Typically, the length of neovessels generated from the limbal vessel ring toward the centre of the cornea and the width of the neovessels are measured.

As indicated above, the therapeutic compositions alone or in combination with other anti-cancer therapeutic(s) can be used to attenuate the growth and/or metastasis of a tumour in vivo. A number of standard murine models of cancer known in the art can be used initially to assess the ability of the compositions to attenuate the growth and/or metastasis of tumours (see, for example, Enna, et al., Current Protocols in Pharmacology, J. Wiley & Sons, Inc., New York, N.Y.).

In general, current animal models for screening anti-tumour compounds are xenograft models, in which a human tumour has been implanted into a mouse. Examples of xenograft models of human cancer include, but are not limited to, human solid tumour xenografts, implanted by sub-cutaneous injection or implantation and used in tumour growth assays; human solid tumour isografts, implanted by fat pad injection and used in tumour growth assays; human solid tumour orthotopic xenografts, implanted directly into the relevant tissue and used in tumour growth assays; experimental models of lymphoma and leukaemia in mice, used in survival assays, and experimental models of lung metastasis in mice. Non-limiting examples of cancer cell lines that can be used in these assays are provided in Table 2.

TABLE 2
Examples of Xenograft Models of Cancer

| Cancer Model | Cell Type | |
|---|---|---|
| Tumour Growth Assay | Human: | Prostate (PC-3, DU145) |
| Human solid tumour xenografts in | | Breast (MDA-MB-231, MVB-9) |
| mice (sub-cutaneous injection) | | Colon (HT-29) |
| | | Lung (NCI-H460, NCI-H209) |
| | | Pancreatic (ASPC-1, SU86.86) |
| | | Pancreatic: drug resistant (BxPC-3) |
| | | Skin (melanoma: A2058, C8161) |
| | | Cervical (SIHA, HeLa-S3) |
| | | Cervical: drug resistant (HeLa S3- |
| | | HU-resistance) |
| | | Liver (HepG2) |
| | | Brain (U87-MG) |
| | | Renal (Caki-1, A498) |
| | | Ovary (SK-OV-3) |
| | Murine: | Melanoma (B16F10) |

| | |
|---|---|
| Tumour Growth Assay | Breast: drug resistant (MDA-CDDP-S4, |
| Human solid tumour isografts in | MDA-MB435-To.1) |
| mice (fat pad injection) | |
| Survival Assay | Human: Burkitts lymphoma (Non-Hodgkin's) |
| Experimental model of lymphoma | (raji) |
| and leukaemia in mice | Murine: erythroleukemia (CB7 Friend |
| | retrovirus-induced) |
| Experimental model of lung | Human: melanoma (C8161) |
| metastasis in mice | Murine: fibrosarcoma (R3) |
| | Murine: Lewis lung carcinoma |

For example, the compositions can be tested in vivo on solid tumours using mice that are subcutaneously grafted bilaterally with 30 to 60 mg of a tumour fragment, or implanted with an appropriate number of cancer cells, on day 0, Subcutaneous xenografts metastasize infrequently and seldom invade adjacent tissue, therefore, rate of tumour growth or delay of significant tumour growth are the endpoints used in this model. The animals bearing tumours are mixed before being subjected to the various treatments and controls. In the case of treatment of advanced tumours, tumours are allowed to develop to the desired size, animals having insufficiently developed tumours being eliminated. The selected animals are distributed at random to undergo the treatments and controls. Suitable controls will be dependent on the actual composition being tested and whether or not the composition is being evaluated in combination with a chemotherapeutic. Thus, for example, for testing a composition that comprises two plant extracts suitable controls could include animals receiving each of the extracts alone, animals receiving standard chemotherapy and untreated animals. Testing a composition in combination with a chemotherapeutic could include control animals receiving effective doses and sub-effective doses of the chemotherapeutic, animals receiving the plant extract(s) alone as well as untreated animals. Animals not bearing tumours may also be subjected to the same treatments as the tumour-bearing animals in order to be able to dissociate the toxic effect from the specific effect on the tumour. Experiments to test the efficacy of various compositions and combinations can readily be designed by a skilled technician.

Chemotherapy generally begins from 1 to 22 days after grafting, depending on the type of tumour, and the animals are observed every day. Alternatively, to evaluate the preventative properties of the compositions, the composition can be administered prior to tumour implantation, for example, about 7 days prior. The compositions of the present invention can be administered to the animals, for example, orally, by i.p. injection or bolus infusion. Anti-cancer therapeutics, if used, can be administered by similar routes. The different animal groups are weighed about 3 or 4 times a week until the maximum weight loss is attained, after which the groups are weighed at least once a week until the end of the trial.

The tumours are measured after a pre-determined time period, or they can be monitored continuously by measuring about 2 or 3 times a week until the tumour reaches a pre-determined size and/or weight, or until the animal dies if this occurs before the tumour reaches the pre-determined size/weight. The animals are then sacrificed and the tissue histology, size and/or proliferation of the tumour assessed.

Orthotopic xenograft models are an alternative to subcutaneous models and may more accurately reflect the cancer development process. In this model, tumour cells are implanted at the site of the organ of origin and develop internally. Daily evaluation of the size of the tumours is thus more difficult than in a subcutaneous model. A recently developed technique using green fluorescent protein (GFP) expressing tumours in non-invasive whole-body imaging can help to address this issue (Yang and al, Proc. Nat. Aca. Sci, (2000), pp 1206-1211). This technique utilises human or murine tumours that stably express very high levels of the Aqueora vitoria green fluorescent protein. The GFP expressing tumours can be visualised by means of externally placed video detectors, allowing for monitoring of details of tumour growth, angiogenesis and metastatic spread. Angiogenesis can be measured over time by monitoring the blood vessel density within the tumour(s). The use of this model thus allows for simultaneous monitoring of several features associated with tumour progression and has high preclinical and clinical relevance.

For the study of the effect of the compositions on leukaemias, the animals are grafted with a particular number of cells, and the anti-tumour activity is determined by the increase in the survival time of the treated mice relative to the controls.

To study the effect of the compositions of the present invention on tumour metastasis, various models of experimental metastasis known in the art can be employed. Typically, this involves the treatment of neoplastic cells with the extract ex vivo and subsequent injection or implantation of the cells into a suitable test animal. Alternatively, the animals are treated before or after injection or implantation of the neoplastic cells into the animal. The spread of the neoplastic cells from the site of injection, for example spread to the lungs and/or lymphoid nodes, is then monitored over a suitable period of time by standard techniques.

An alternative in vivo model of metastasis utilises highly metastatic, chemotherapy-resistant cultured Lewis lung (LLC1) cells. The cells are administered intravenously to normal non-immune-compromised mice thus allowing for immediate dissemination of cancerous cells. Treatment can be initiated several days before injection of the LLC1 cells in order to observe a preventive effect or immediately after injection of the cells in order to observe an attenuating effect. After about 14 days, the mice are sacrificed, the lungs removed and fixed and the number and size of lung tumours determined. The intravenous route of administration for the LLC1 cells in this model allows for rapid evaluation of treatments.

In another model, LLC1 cells are injected subcutaneously to allow the growth of a primary tumour, which is then surgically removed once a certain size is obtained. Following removal of the primary tumour, treatment is initiated for about 14 days, after which the animals are sacrificed and tumours counted as in the intravenous model. The primary tumour is removed in this model is recommended as it can be metastasis-suppressing.

When a therapeutic combination of the invention is evaluated utilising this model, a lower (sub-optimal) dose of the chemotherapeutic can be evaluated with and without the therapeutic composition in order to evaluate potential therapeutic synergy between the two treatments and/or the ability of the therapeutic composition to potentiate sub-optimal doses of a chemotherapeutic. Similarly, for compositions comprising more than one extract, each extract can optionally be evaluated separately in order to evaluate potential therapeutic synergy.

In vivo toxic effects of the compositions can also be evaluated from the above experiments by measuring their effect on animal body weight during treatment and by performing haematological profiles and liver enzyme analysis after the animal has been sacrificed. Alternatively, separate tests to evaluate the toxicity of the extracts or compositions can be conducted.

### 3. Additional Tests

In addition to the above tests, the therapeutic compositions of the invention can be submitted to other standard tests, such as cytotoxicity tests, stability tests, bioavailability tests and the like. As will be readily apparent to one skilled in the art, the therapeutic compositions of the invention will need to meet certain criteria in order to be suitable for human or animal use and to meet regulatory requirements. Thus, once a composition of the invention has been found to be suitable for animal administration, standard in vitro and in vivo tests can be conducted to determine information about the metabolism and pharmacokinetics (PK) of the compositions, including data on drug-drug interactions where appropriate, which can be used to design human clinical trials. Toxicity and dosing information can likewise be obtained through standard pre-clinical evaluations. Appropriate dosages can be readily determined from such pre-clinical data and, when necessary, the therapeutic compositions can be evaluated for their efficacy in standard clinical trials procedures such as those described below.

### 4. Therapeutic Effect of Combination Therapies

In accordance with one embodiment of the present invention, the therapeutic compositions are used in combination with one or more standard anti-cancer therapeutics in the treatment of cancer. Such combinations of a therapeutic composition of the invention with one or more anti-cancer therapeutics have an improved therapeutic effect compared to the therapeutic effect of each of the individual components of the combination when administered alone.

An improved therapeutic effect can be manifested, for example, as an increase in the efficacy of the one or more component of the composition/combination in attenuating tumour growth and/or metastasis and/or a decrease or delay in the toxicity phenomena associated with one or more component.

An improved therapeutic effect can be measured, for example, by determining whether the combination of components results in an improved therapeutic index compared to each of the individual components.

The ratio of the median effective dose (ED₅₀) and the median lethal dose (LD₅₀) can be used as an indication of the therapeutic index of a compound. The ED₅₀ of a drug is the dose required to produce a specified effect in 50% of a test population and the LD₅₀ of a drug is the dose that has a lethal effect on 50% of a test population. The LD₅₀ is determined in preclinical trials, whereas the ED₅₀ can be tested in preclinical or clinical trials. Alternatively the therapeutic index can be determined based on doses that produce a therapeutic effect and doses that produce a toxic effect (for example, ED₉₀ and LD₁₀, respectively). During clinical studies, the dose, or the concentration (for example, in solution in blood, serum, or plasma), of a drug required to produce toxic effects can be compared to the concentration required for the therapeutic effects in the population to evaluate the clinical therapeutic index. Methods of clinical studies to evaluate the clinical therapeutic index are well known to workers skilled in the art.

In one embodiment of the present invention, use of a combination results in an improved LD₅₀ for at least one of the components in the combination. In another embodiment use of a combination results in an improved ED₅₀ for at least one of the components in the combination.

An improved therapeutic effect can also be manifested as therapeutic synergy. A combination manifests therapeutic synergy when it is therapeutically superior to one of the components when used at that component's optimum dose [T. H. Corbett et al, (1982) Cancer Treatment Reports, 66, 1187]. To demonstrate the efficacy of a combination, it may be necessary to compare the maximum tolerated dose of the combination with the maximum tolerated dose of each of the separate components in the study in question. This efficacy may be quantified using techniques and equations commonly known to workers skilled in the art. [T. H. Corbett et al., (1977) Cancer, 40, 2660.2680; F. M. Schabel et al., (1979) Cancer Drug Development, Part B, Methods in Cancer Research, 17, 3-51, New York, Academic Press Inc.].

The combination, used at its own maximum tolerated dose, in which each of the components will be present at a dose generally not exceeding its maximum tolerated dose (MTD), will manifest therapeutic synergy when the efficacy of the combination is greater than the efficacy of the best component when it is administered alone. In one embodiment of the present invention, at least one component of the combination is used at less than its MTD. In another embodiment of the invention, the combination comprises a chemotherapeutic drug that is used at less than its MTD.

Thus, in one embodiment of the present invention, in order to prepare a therapeutic combination, one or more plant extract is first selected and the efficacy of the extract(s) in attenuating the growth and/or metastasis of a tumour is determined using standard techniques, such as those outlined above. The efficacy of the one or more plant extract alone is then compared to the efficacy of the one or more plant extract in combination with varying amounts of another component, i.e. another plant extract, synthetic inhibitor or anti-cancer therapeutic. A combination that demonstrates therapeutic synergy or an improved therapeutic index in comparison to the individual components is considered to be an effective combination.

### Commercial Processes for Preparing Plant Extracts of the Invention

The present invention contemplates the large-scale preparation of selected plant extracts of the invention. Such extracts can be prepared on a commercial scale by repeating the extraction process that lead to the isolation of the extract of interest. One embodiment of this aspect of the invention is presented in FIG. 5. In this embodiment, the small-scale extraction procedure is simply scaled-up and additional steps of quality control are included to ensure reproducible results for the resulting extracts. Similarly the process outlined in FIG. 4 can be scaled up for commercial purposes.

Also contemplated by the present invention are modifications to the small-scale procedure that may be required during scale-up for industrial level production of the extract. Such modifications include, for example, alterations to the solvent being used or to the extraction procedure employed in order to compensate for variations that occur during scale-up and render the overall procedure more amenable to industrial scale production, or more cost effective. Modifications of this type are standard in the industry and would be readily apparent to those skilled in the art.

Purification/Fractionation of Active Ingredients from Extracts of the Invention

The present invention also provides for purified/semi-purified active ingredients isolated from the plant extracts of the invention. In the context of the present invention an “active ingredient” is a compound that is capable of inhibiting MMP-9 or cathepsin B. The compound may be either proteinaceous or non-proteinaceous.

There are a number of techniques well known in the art for isolating active ingredients from mixtures. For example, purification, partial purification, and/or fractionation can be performed using solid-liquid extraction, liquid-liquid extraction, solid-phase extraction (SPE), membrane filtration, ultrafiltration, dialysis, electrophoresis, solvent concentration, centrifugation, ultracentrifugation, liquid or gas phase chromatography (including size exclusion, affinity, etc.) with or without high pressure, lyophilisation, evaporation, precipitation with various “carriers” (including PVPP, carbon, antibodies, etc.), or various combinations thereof. Such techniques are described in Section 1.1.4. above and are suitable for use in the purification, partial purification, and/or fractionation of active ingredients from an extract of the invention.

Thus an extract of the invention can be subjected to one or more of the above techniques, in a sequential fashion, in order to obtain a substantially purified compound, or compounds, therefrom that retains the activity of interest (i.e. the ability to inhibit MMP-9 and/or cathepsin B activity). Purified, partially purified and/or concentrated compounds can be tested for their ability to inhibit MMP-9 and/or cathepsin B according to one or more of the procedures described above. Furthermore, and where identification and/or quantification of key fractions or purified phytochemicals of the extracts of the invention is desired, analytical techniques including, but not limited to, NMR, GC-MS, TLC, spectrophotometry, microspray, X-ray diffraction and elemental analysis may be performed to elucidate the active components or fractions of the extract.

### Pharmaceutical and Naturopathic Formulations

For administration to a mammal, the therapeutic compositions can be formulated as pharmaceutical or naturopathic formulations such as phytoceuticals or nutraceuticals, for oral, topical, rectal or parenteral administration or for administration by inhalation or spray. The pharmaceutical/naturopathic formulations comprise the one or more plant extracts in dosage unit formulations containing conventional non-toxic physiologically acceptable carriers, adjuvants and vehicles. The term parenteral as used herein includes subcutaneous injections, intravenous, intramuscular, intrathecal, intrasternal injection or infusion techniques.

The formulations of the present invention contain at least an effective amount of the therapeutic composition. The effective amount is considered to be that amount of the composition, in weight percent of the overall formulation, which must be present in order to produce the desired therapeutic effect. As would be apparent to one skilled in the art, the effective amount may vary, depending upon, for example, the disease to be treated and the form of administration. In general, the therapeutic composition will be present in an amount ranging from about 1% to about 100% by weight of the formulation. In one embodiment of the present invention, the therapeutic composition is present in an amount ranging from about 10% to about 90% by weight of the formulation. In another embodiment, the therapeutic composition is present in an amount ranging from about 20% to about 80% by weight. In other embodiments, the therapeutic composition is present in an amount ranging from about 30% to about 70% by weight, from about 40 to about 60% by weight, and about 50% by weight of the formulation.

The pharmaceutical/naturopathic formulations may be in a form suitable for oral use, for example, as tablets, troches, lozenges, aqueous or oily suspensions, dispersible powders or granules, emulsion hard or soft capsules, or syrups or elixirs. The therapeutic compositions of the invention may be formulated as phytoceuticals, or nutraceuticals. Phytoceuticals may optionally comprise other plant-derived components and can therefore be delivered by such non-limiting vehicles as teas, tonics, juices or syrups. Nutraceuticals contemplated by the present invention may provide nutritional and/or supplemental benefits and can therefore be delivered, for example, as foods, dietary supplements, extracts, beverages or the like. Phytoceuticals and nutraceuticals can be administered in accordance with conventional treatment programs, naturopathic treatment programs, and or may from part of a dietary or supplemental program.

Formulations intended for oral use may be prepared according to methods known to the art for the manufacture of pharmaceutical compositions and may contain one or more agents selected from the group of sweetening agents, flavouring agents, colouring agents and preserving agents in order to provide palatable preparations. Tablets contain the active ingredient in admixture with suitable non-toxic physiologically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Various additives or carriers can be incorporated into the orally delivered pharmaceutical/naturopathic formulations or the invention. Optional additives of the present composition include, without limitation, phospholipids, such as phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins. Bioactive agent delivery particles including bilayer-forming and non-bilayer-forming lipids are also contemplated. Such lipids include phospholipids, dimyristoylphosphatidylcholine (DMPC) and dimyristoylphosphatidylglycerol (DMPG). Inclusion of apolipoprotein is also contemplated.

Pharmaceutical/naturopathic formulations for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the plant extract(s) in admixture with suitable excipients including, for example, suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the plant extract(s) in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These formulations can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical/naturopathic formulations of the invention may also be in the form of oil-in-water emulsions. The oil phase may be a vegetable oil, for example, olive oil or arachis oil, or a mineral oil, for example, liquid paraffin, or it may be a mixtures of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the partial esters with ethylene oxide, for example, polyoxyethylene sorbitan monoleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents.

The pharmaceutical/naturopathic formulations may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils, which are conventionally employed as a solvent or suspending medium, and a variety of bland fixed oils including, for example, synthetic mono- or diglycerides. In addition, fatty acids such as oleic acid find use in the preparation of injectables.

Other pharmaceutical formulations and methods of preparing the same are known in the art and are described, for example, in “Remington: The Science and Practice of Pharmacy” (formerly “Remington Pharmaceutical Sciences”); Gennaro, A., Lippincott, Williams & Wilkins, Philadelphia, Pa. (2000).

### Use of the Therapeutic Compositions

The present invention further provides for the use of therapeutic compositions for the targeted inhibition of MMP-9 and cathepsin B in the treatment of cancer. The therapeutic compositions may be used alone or in combination with one or more anti-cancer agents to inhibit one or more of neoplastic cell migration, endothelial cell migration, tumour growth, tumour metastasis, and tumour-induced angiogenesis.

The present invention further contemplates that where toxicity is a factor, for example, in patients that cannot tolerate optimal or standard chemotherapeutic doses (such as, obese or elderly patients), or in cases where the patient's metabolism is compromised (such as, individuals suffering from liver disease or disorder), the therapeutic compositions can be used in combination with sub-optimal doses of known anti-cancer therapeutic(s).

### 1. Methods of Treating Cancer

The present invention contemplates methods of treating cancer by administering an effective amount of a therapeutic composition which simultaneously inhibits MMP-9 and cathepsin B. The therapeutic compositions of the invention can be administered alone or in combination with one or more standard anti-cancer therapeutics for the treatment of cancer. The present invention further provides for methods of treating cancer by administration of sub-optimal doses of the anti-cancer therapeutic(s), for example, chemotherapeutic drug(s), in combination with the therapeutic composition. In this context, treatment with a composition of the invention may result in, for example, a reduction in the size of a tumour, the slowing or prevention of an increase in the size of a tumour, a reduction in tumour vascularisation, a reduction in tumour metastasis, a slowing or prevention of an increase in metastasis, an increase in the disease-free survival time between the disappearance or removal of a tumour and its reappearance, prevention of an initial or subsequent occurrence of a tumour (e.g. metastasis), an increase in the time to progression, reduction of one or more adverse symptom associated with a tumour, or an increase in the overall survival time of a subject having cancer.

In accordance with a further embodiment of the present invention, there is provided a method of treating cancer in a subject by administering to the subject effective amounts of a MMP-9 inhibitor in combination with a cathepsin B inhibitor. The inhibitors can be one or more plant extracts, or compounds purified therefrom, or they can be synthetic MMP-9 and cathepsin B inhibitors, or combinations thereof. Suitable synthetic MMP-9 and cathepsin B inhibitors include those known in the art and currently available, such as marimastat, prinomastat, tanomastat, metastat, E-64, CA-074 methyl-ester, leupeptin, 1-phenyl-1,4-epoxy-1H,4H-naphtho[1,8-de][1,2]dioxepin (ANO-2) and ilomastat (also known as N-[(2R)-2-(hydroxamidocarbonylmethyl)-4-methylpentanoyl]-L-tryptophan methylamide, Galardin™ or GM-6001). Other synthetic inhibitors that may be developed in the future are also suitable for use in the methods of the present invention.

### 1.1 Cancer Types

The therapeutic compositions of the invention can be used for the treatment of a variety of tumours. Exemplary tumours include, but are not limited to, haematologic neoplasms, including leukaemias and lymphomas; carcinomas, including adenocarcinomas; melanomas and sarcomas. Carcinomas, adenocarcinomas and sarcomas are also frequently referred to as “solid tumours,” examples of commonly occurring solid tumours include, but are not limited to, cancer of the brain, breast, cervix, colon, head and neck, kidney, lung, ovary, pancreas, prostate, stomach and uterus, non-small cell lung cancer and colorectal cancer. Various forms of lymphoma also may result in the formation of a solid tumour and, therefore, are also often considered to be solid tumours.

The term “leukaemia” refers broadly to progressive, malignant diseases of the blood-forming organs. Leukaemia is typically characterized by a distorted proliferation and development of leukocytes and their precursors in the blood and bone marrow but can also refer to malignant diseases of other blood cells such as erythroleukaemia, which affects immature red blood cells. Leukaemia is generally clinically classified on the basis of (1) the duration and character of the disease—acute or chronic; (2) the type of cell involved—myeloid (myelogenous), lymphoid (lymphogenous) or monocytic, and (3) the increase or non-increase in the number of abnormal cells in the blood—leukaemic or aleukaemic (subleukaemic). Leukaemia includes, for example, acute nonlymphocytic leukaemia, chronic lymphocytic leukaemia, acute granulocytic leukaemia, chronic granulocytic leukaemia, acute promyelocytic leukaemia, adult T-cell leukaemia, aleukaemic leukaemia, aleukocythemic leukaemia, basophylic leukaemia, blast cell leukaemia, bovine leukaemia, chronic myelocytic leukaemia, leukaemia cutis, embryonal leukaemia, eosinophilic leukaemia, Gross' leukaemia, hairy-cell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenous leukaemia, lymphoid leukaemia, lymphosarcoma cell leukaemia, mast cell leukaemia, megakaryocytic leukaemia, micromyeloblastic leukaemia, monocytic leukaemia, myeloblastic leukaemia, myelocytic leukaemia, myeloid granulocytic leukaemia, myelomonocytic leukaemia, Naegeli leukaemia, plasma cell leukaemia, plasmacytic leukaemia, promyelocytic leukaemia, Rieder cell leukaemia, Schilling's leukaemia, stem cell leukaemia, subleukaemic leukaemia, and undifferentiated cell leukaemia.

The term “lymphoma” generally refers to a malignant neoplasm of the lymphatic system, including cancer of the lymphatic system. The two main types of lymphoma are Hodgkin's disease (HD or HL) and non-Hodgkin's lymphoma (NHL). Abnormal cells appear as congregations which enlarge the lymph nodes, form solid tumours in the body, or more rarely, like leukemia, circulate in the blood. Hodgkin's disease lymphomas, include nodular lymphocyte predominance Hodgkin's lymphoma; classical Hodgkin's lymphoma; nodular sclerosis Hodgkin's lymphoma; lymphocyte-rich classical Hodgkin's lymphoma; mixed cellularity Hodgkin's lymphoma; lymphocyte depletion Hodgkin's lymphoma. Non-Hodgkin's lymphomas include small lymphocytic NHL, follicular NHL; mantle cell NHL; mucosa-associated lymphoid tissue (MALT) NHL; diffuse large cell B-cell NHL; mediastinal large B-cell NHL; precursor T lymphoblastic NHL; cutaneous T-cell NHL; T-cell and natural killer cell NHL; mature (peripheral) T-cell NHL; Burkitt's lymphoma; mycosis fungoides; Sézary Syndrome; precursor B-lymophoblastic lymphoma; B-cell small lymphocytic lymphoma; lymphoplasmacytic lymphoma; spenic marginal zome B-cell lymphoma; nodal marginal zome lymphoma; plasma cell myeloma/plasmacytoma; intravascular large B-cell NHL; primary effusion lymphoma; blastic natural killer cell lymphoma; enteropathy-type T-cell lymphoma; hepatosplenic gamma-delta T-cell lymphoma; subcutaneous panniculitis-like T-cell lymphoma; angioimmunoblastic T-cell lymphoma; and primary systemic anaplastic large T/null cell lymphoma.

The term “sarcoma” generally refers to a tumour which originates in connective tissue, such as muscle, bone, cartilage or fat, and is made up of a substance like embryonic connective tissue and is generally composed of closely packed cells embedded in a fibrillar or homogeneous substance. Sarcomas include soft tissue sarcomas, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumour sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented haemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumour arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colorectal carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, haematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, non-small cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “carcinoma” also encompasses adenocarcinomas. Adenocarcinomas are carcinomas that originate in cells that make organs which have glandular (secretory) properties or that originate in cells that line hollow viscera, such as the gastrointestinal tract or bronchial epithelia. Examples include, but are not limited to, adenocarcinomas of the breast, lung, pancreas and prostate.

Additional cancers encompassed by the present invention include, for example, multiple myeloma, neuroblastoma, rhabdomyosarcoma, primary thrombocytosis, primary macroglobulinemia, small-cell lung tumours, primary brain tumours, malignant pancreatic insulanoma, malignant carcinoid, urinary bladder cancer, premalignant skin lesions, gliomas, testicular cancer, thyroid cancer, esophageal cancer, genitourinary tract cancer, malignant hypercalcemia, endometrial cancer, adrenal cortical cancer, mesothelioma and medulloblastoma.

The cancer to be treated may be indolent or it may be aggressive. The present invention contemplates the use of the therapeutic compositions in the treatment of refractory cancers, advanced cancers, recurrent cancers and metastatic cancers. One skilled in the art will appreciate that many of these categories may overlap, for example, aggressive cancers are typically also metastatic.

“Aggressive cancer,” as used herein, refers to a rapidly growing cancer. One skilled in the art will appreciate that for some cancers, such as breast cancer or prostate cancer the term “aggressive cancer” will refer to an advanced cancer that has relapsed within approximately the earlier two-thirds of the spectrum of relapse times for a given cancer, whereas for other types of cancer, such as small cell lung carcinoma (SCLC) nearly all cases present rapidly growing cancers which are considered to be aggressive. The term can thus cover a subsection of a certain cancer type or it may encompass all of other cancer types. A “refractory” cancer or tumour refers to a cancer or tumour that has not responded to treatment. “Advanced cancer,” refers to overt disease in a patient, wherein such overt disease is not amenable to cure by local modalities of treatment, such as surgery or radiotherapy. Advanced disease may refer to a locally advanced cancer or it may refer to metastatic cancer. The term “metastatic cancer” refers to cancer that has spread from one part of the body to another. Advanced cancers may also be unresectable, that is, they have spread to surrounding tissue and cannot be surgically removed.

The therapeutic compositions may also be used to treat drug resistant cancers, including multidrug resistant tumours. As is known in the art, the resistance of cancer cells to chemotherapy is one of the central problems in the management of cancer.

Certain cancers, such as prostate and breast cancer, can be treated by hormone therapy, i.e. with hormones or anti-hormone drugs that slow or stop the growth of certain cancers by blocking the body's natural hormones. Such cancers may develop resistance, or be intrinsically resistant, to hormone therapy. The present invention further contemplates the use of the therapeutic compositions in the treatment of such “hormone-resistant” or “hormone-refractory” cancers.

The present invention also contemplates the use of the compositions as “sensitizing agents.” In this case, the composition alone does not have a cytotoxic effect on the cancer cells, but provides a means of weakening the cells, and thereby facilitates the benefit from conventional anti-cancer therapeutics.

### 1.2 Administration

The present invention contemplates the administration of an effective amount of a therapeutic composition of the invention to a subject, alone or in combination with one or more standard anti-cancer therapeutics, for the treatment or prevention of cancer. In the context of the present invention, “prevention of cancer” includes the prevention of the first occurrence of a tumour in an individual, for example an individual at risk of developing cancer, as well as the prevention of recurrence of a cancer in a patient, or the relapse of patient, after one or more other therapeutic interventions.

The present invention contemplates the use of the therapeutic compositions at various stages in tumour development and progression, including in the treatment of early stage, or advanced and/or aggressive neoplasias, metastatic disease, locally advanced disease and/or refractory tumours.

Thus, the compositions and combinations can be administered to a patient after initial diagnosis, i.e. as part of a neo-adjuvant therapy (to primary therapy). Exemplary primary therapies involve surgery, a wide range of chemotherapies and radiotherapy. The intention of primary therapy can be to remove the tumour (in the case of surgery) or to delay progression and/or metastasis of the disease.

The present invention contemplates that the therapeutic compositions can be administered to a mammal having early stage cancer to help attenuate the progression of the disease through their effect on tumour growth and/or metastasis. The latter effect is particularly useful in further slowing down a cancer that progresses relatively slowly, such as prostate cancer.

Alternatively, the compositions can be administered to a patient as part of an adjuvant therapy regimen to delay recurrence or relapse, prolong survival or cure a subject. Adjuvant systemic therapy is typically started soon after primary therapy.

It is further contemplated that the compositions can be administered to a patient prophylactically to attenuate the growth or metastasis of a tumour. This application is particularly useful for those patients having an aggressive disease that is known to metastasise readily.

As indicated above, the therapeutic compositions can be used in combination with one or more anti-cancer therapeutics with the intention of improving the efficacy of the anti-cancer therapeutic(s). In this context, the therapeutic composition is considered to be an “adjuvant” to the anti-cancer therapeutic(s). The composition can thus decrease the amount of the anti-cancer therapeutic required to achieve the desired effect and thereby lead to an increased efficacy, decreased side-effects and/or more cost-effective treatment regimens. Alternatively, this approach can be taken in the treatment of drug-resistant cancers unresponsive to standard treatment in order to weaken the tumour with the intention of rendering it susceptible to standard therapeutics. The therapeutic compositions can also be used to potentiate the effect of standard doses of the anti-cancer therapeutic, or to potentiate to effect of sub-optimal doses of the anti-cancer therapeutic in those patients who cannot tolerate standard doses.

When the therapeutic compositions are administered in combination with one or more anti-cancer therapeutics, the components of the composition can be administered together or sequentially. Typically in the treatment of cancer, chemotherapeutic agents are administered systemically to patients, for example, by bolus injection or continuous infusion into a patient's bloodstream. However, chemotherapeutic agents may also be administered orally. The therapeutic composition of the invention can be administered prior to, or after, administration of the therapeutic(s) of the combination, or they can be administered concurrently.

### 1.3 Dosing

The dosage of the therapeutic composition to be administered is not subject to defined limits, but it will usually be an effective amount. Daily dosages of a composition of the present invention will typically fall within the range of about 1 to about 2000 mg/kg of body weight, for example, about 10 to about 1000 mg/kg of body weight, in single or divided dose. However, it will be understood that the actual amount of the composition to be administered will be determined by a physician, in the light of the relevant circumstances, including the condition to be treated, the chosen route of administration, the actual composition administered, the age, weight, and response of the individual patient, and the severity of the patient's symptoms. The above dosage range is given by way of example only and is not intended to limit the scope of the invention in any way. In some instances dosage levels below the lower limit of the aforesaid range may be more than adequate, while in other cases still larger doses may be employed without causing harmful side effects, for example, by first dividing the larger dose into several smaller doses for administration throughout the day.

### 1.4 Sub-Optimal Dosing

For those patients for whom the toxicity associated with standard or optimal anti-cancer therapeutic treatment is intolerable or prohibitive (for example, elderly, overweight or obese patients, metabolically compromised individuals (such as those suffering from liver disease), or individuals suffering from neutropenia), the present invention also contemplates the use of the therapeutic compositions as part of effective alternatives to standard chemotherapeutic therapies. As described herein, use of a therapeutic composition of the invention in combination with one or more anti-cancer therapeutic(s) may result in a greater net therapeutic benefit, as compared with the use of either the therapeutic composition or the anti-cancer therapeutic(s) alone. This enhanced therapeutic index may come about, for example, as a result of the potentiation of an anti-cancer agent(s) by the therapeutic composition of the invention and, in turn, allows for the effective treatment of cancer through the administration of reduced levels of anti-cancer agent(s), in combination with a therapeutic composition of the invention. Accordingly, in one embodiment of the invention there is provided a method for treating cancer by administering to a subject a sub-optimal dose of one or more chemotherapeutic agents in combination with a therapeutic composition of the invention.

As noted above, elderly or overweight subjects, as well as those suffering from obesity, neutropenia or a liver disease or disorder, are suitable candidates for receiving the sub-optimal chemotherapeutic combinations of the invention. However, given that there is no loss in the efficacy associated with the sub-optimal chemotherapeutic combinations, as compared to standard chemotherapeutic therapies, the present invention also contemplates the use of the therapeutic combination of the invention to treat other cancer patients in order, for example, to decrease the side-effects of the standard therapy, allow for fewer administrations of the standard anti-cancer therapeutic and/or provide for more cost-effective treatment regimens.

### Clinical Trials

One skilled in the art will appreciate that, following the demonstrated effectiveness of the therapeutic compositions of the present invention in vitro and in animal models (i.e. pre-clinical efficacy), the safety profile of the compositions can be determined in at least two non-human species and then the compositions may, where necessary, progress into Clinical Trials in order to further evaluate their efficacy in attenuating the growth and/or metastasis of tumours and to obtain regulatory approval for therapeutic use. As is known in the art, clinical trials progress through phases of testing, which are identified as Phases I, II, III, and IV. In vitro and in vivo information about the metabolism and pharmacokinetics (PK) of the compositions, including data on drug-drug interactions where appropriate, determined from pre-clinical studies facilitates the design of initial Phase I and Phase II clinical studies.

### Phase I

Phase I clinical trials are normally performed in healthy human volunteers or in advanced cancer patients. These studies are conducted to investigate the safety, tolerability and PK of the compositions and to help design Phase II studies, for example, in terms of appropriate doses, routes of administration, administration protocols. Phase I studies could incorporate pharmacodynamic assays to evaluate proof of principle in inhibition of target in humans. An adequate pharmacodynamic endpoint would be to determine the inhibitory activity measured from the plasma of healthy volunteers. An exemplary Phase I study could be structured to determine the following information:
1. Safety, tolerance and PK in healthy subjects following single oral dose: a study composed of a suitable number of subjects, which should be a single blind, randomized, placebo controlled study.
2. Safety, tolerance and PK in healthy subjects following repeat dose (14 days): a study composed of a suitable number of subjects, which should be a single blind, randomized, placebo controlled study.
3. Effects on age, gender or other co-administered drugs on safety, tolerance and PK.

For combinations of a therapeutic composition of the invention with one or more anti-cancer therapeutics, placebo controlled confirmatory studies may need to be conducted in normal volunteers to study the PK modulation of the therapeutic composition when used in combination with a first-line chemotherapeutic agent. Variation in the PK of the first-line chemotherapeutic agent may also need to be investigated.

### Phase II

Phase I studies allow the selection of safe dose levels for Phase II studies. An important factor in the protocol design of the Phase II studies is the adequate recruitment of the patient population to be studied based on stringent selection criteria defining the demographics (age, race and sex) of the study, the previous medical history of the patient, the type of cancer and stage of its development as well as any previous cancer treatment history. The latter factor can be important when the composition is intended as an adjuvant to first line therapy rather than a treatment to refractory disease. A protocol for Phase II studies typically specifies baseline data that can be used to characterise the population, to evaluate the success of randomization in achieving balance of important prognostic factors, and to allow for consideration of adjusted analyses.

### Staging of the Cancers of Interest

Staging of the cancer being investigated can be important and, when possible, patients should be recruited such that the cancer stage is as homogeneous as possible across the population to facilitate statistical analysis and interpretation of the data. As is known in the art, methods and criteria for staging of a cancer vary depending on the particular cancer being investigated.

By way of example, for prostate cancer, initial staging is related to histologic evaluation of biopsies (TNM system; see Table 3). These biopsies are recommended according to blood prostate specific antigen (PSA) level, which is routinely monitored in patients at risk. According to the American Urological Society, the risk of cancer associated with increasing PSA levels is as follows:

PSA under 4 ng/mL: normal
PSA 4 to 10 ng/mL: 20 to 30% risk
PSA 10 to 20 ng/mL: 50 to 75% risk
PSA above 20 ng/mL: 90%

TABLE 3
Prostate cancer staging, TNM System

| Stage | Characteristic |
|---|---|
| T1a | Tumour incidental histologic finding less than or equal to 5% |
| | of resected tissue; not palpable; well differentiated |
| T1b | Tumour incidental histologic finding greater than 5% of |
| | resected tissue, moderately to poorly differentiated |
| T1c | Tumour identified by needle biopsy |
| T2a | Tumour involves one lobe |
| T2b | Tumour involves both lobes |
| T3a | Extracapsular extension (unilateral or bilateral) |
| T3b | Tumour invades seminal vesicle(s) |
| T4 | Bladder invasion, adhesion to pelvic side wall, or invasion of |
| | adjacent structures |

Staging of colorectal cancer in clinical studies is particularly important due to the wide variability in the rate of progression of this cancer. Unlike other cancers, staging of colorectal cancer is not related to the size of tumour but to the depth of penetration of the tumour into the bowel wall, which involves proteolysis. A staging system for colorectal cancer has been suggested in the American Joint Committee on Cancer Manual for staging of Cancer (AJCC): 2nd ed. Hagerstown Md., Lippincot (1983) and is presented in Table 4. Subjects for phase II clinical trials with a composition of the invention could include, for example, subjects who are at the point of chemotherapeutic intervention.

TABLE 4
Carcinoma of the colorectum staging: AJCC (1983)

| Stage | Characteristic |
|---|---|
| 0 | Carcinoma in situ |
| Ia | Tumour confined to mucosa and submucosa |
| Ib | Tumour involves muscularis propria but not beyond |
| II | Invasion of all layers of bowel wall with or without invasion of |
| | immediately adjacent structures |
| III | Any degree of bowel wall involvement with regional node |
| | metastasis |
| | OR: Extends beyond contiguous tissue with no regional lymph |
| | node metastasis |
| IV | Any invasion of bowel wall with or without regional lymph node |
| | metastasis but with evidence of distant metastasis |

For brain cancer, no formal staging system exists since brain cancer cannot be staged in the same way as other cancers. Initial diagnosis usually follows symptoms reported by the patient. When histology is possible, the primary brain tumour can be staged as Grade Ito IV (see Table 5), with severity frequently being related to the potential of the type of cells diagnosed to spread to other parts of the brain.

TABLE 5
Primary Brain Tumour Staging: World Health Organization Grading
system

| Grade | Characteristic |
|---|---|
| I | The least malignant, usually associated with long-term survival, |
| | slow-growth. Examples include: pilocyticastrocytoma, |
| | craniopharyngioma |
| II | Slow growth, abnormal microscopic appearance, can invade |
| | adjacent tissue and might recur at a higher grade after surgical |
| | removal. |
| III | Malignant tumours, infiltrate adjacent normal brain tissue, tend |
| | to recur often as a higher grade. |
| IV | The most malignant infiltrate widely, with blood vessels and |
| | areas of necrosis. Example: glioblastoma multiforme |

### Clinical Biomarkers

Selection of a clinical biomarker for evaluation of efficacy and/or prediction of outcome (including toxicity) is important for Phase II studies, often this clinical biomarker can be used as a selection criteria for inclusion of patient in the Phase II studies.

Clinical biomarkers can be defined as follows (Atkinson A et al: Clin. Pharmacol. Ther. 69, 89-95 (2001):

Biological marker (biomarker): a characteristic that is objectively measured and evaluated as an indicator of normal biological process, pathogenic process, or pharmacological response to a therapeutic intervention.
Clinical endpoint a characteristic or variable that reflects how a patient feels or functions, or how long a patient survives.
Surrogate endpoint: biomarker intended to substitute for a clinical endpoint. A clinical investigator uses epidemiological, therapeutic, pathophysiological, or other scientific evidence to select a surrogate endpoint that is expected to predict benefit, harm or the lack of benefit or harm. The FDA defines a surrogate endpoint, or marker, as a laboratory measurement or physical sign that is used in therapeutic trials as a substitute for a clinically meaningful endpoint that is a direct measure of how a patient feels, functions or survive and is expected to predict the effect of the therapy.

Biochemical biomarkers have long contributed to the assessment of risk and benefits in cancer and routine clinical assays are available for such markers as prostate-specific antigen and carcinoembryogenic antigen (Grizzle, W E et al, Arch. Pathol. Lab. Med. 125, 91-98, 2001). More recently, imaging of tumour size has gained acceptance (Therasse P et al, J. Natl. Cancer Inst. 92, 205-216, 2000) and this can be of particular importance for protease inhibition. Multi-dimensional imaging adds precision, whereas multi-modal imaging such as positron emission tomography-computed tomography (PET-CT) may allow for quantification of metabolic activity or receptor status. As compared with biopsies and biochemical biomarkers, imaging methods offer the benefit of staging or quantifying therapeutic response, both for single tumours and for global tumour burden, which can be a good broad clinical biomarker.

The potential use of biomarkers is related to the issue of patient selection, where the markers will also be applied to establish baseline values. Previous trials designed for MMP inhibitors may not have been optimally designed and were often targeted at advanced tumours (see, Coussens L M et al, Science 2002, 295:2387-2392; Chantrain C et DeClerck YA, Medecines/Science 2002, 18:565-75; and Overall M O and Lopez-Otin, Nature Reviews, 2002, 2:657-672). Future clinical trials could, therefore, include patients with early diagnosed cancers (nascent tumours) or patients in remission, this would be particularly relevant to cancers such as breast, prostate, melanoma and colorectal cancers for which detection methods are in place.

### Controls

As there are currently no marketed MMP-9, Cathepsin B, or angiogenesis inhibitors that can be used for comparison purposes in a control group, initial trials may need to be designed as placebo-controlled combination therapy trials, where one group would be allocated to receive a standard therapeutic plus a placebo and the second group to receive combination therapy comprising the therapeutic composition of the invention and a standard therapeutic. A positive outcome for a first Phase II would be a good safety profile combined with improvement of a well-defined oncology endpoint (such as lack of progression or regression as demonstrated by tumour imaging). The toxicity profile of the therapeutic combination could be gauged in function of the benefit of the therapy and compared to the toxicity profile of the standard first-line therapy (placebo group). Enhanced toxicity in the treated group could lead to decreased doses of the novel therapy in subsequent trials or to a reduced dose of the first-line chemotherapeutics if a favourable effect on tumour progression is observed during the combination therapy.

### Phase III

Phase III trials focus on determining how the therapeutic composition or combination compares to the standard, or most widely accepted, treatment. In Phase III trials, patients are randomly assigned to one of two or more “arms”. In a trial with two arms, for example, one arm will receive the standard treatment (control group) and the other arm will be treated with the therapeutic composition/combination (investigational group).

### Phase IV

Phase IV trials can be used to further evaluate the long-term safety and effectiveness of the composition. Phase IV trials are less common than Phase I, II and III trials and would take place after the therapeutic composition has been approved for standard use.

### Kits

The present invention additionally provides for therapeutic kits comprising the therapeutic compositions for use in the treatment, stabilization and/or prevention of cancer. Such kits can be pharmaceutical kits intended for use in the clinic or under the guidance of a physician, or they can be naturopathic kits that can be used with or without medical supervision. The kits may additionally comprise one or more other anti-cancer therapeutics or naturopathic preparations for use in combination with the therapeutic compositions of the invention.

Individual components of the kit would be packaged in separate containers and, associated with such containers, can be, when required, instructions and/or a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a patient or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye-dropper or other such medically approved delivery vehicle.

To gain a better understanding of the invention described herein, the following examples are set forth. It should be understood that these examples are for illustrative purposes only. Therefore, they should not limit the scope of this invention in any way.

### EXAMPLES

### Example I

### Preparation of Stressed and Non-Stressed Plant Extracts (Method A)

Pre-Harvest Treatment: Aerial parts of a living plant were sprayed with an aqueous solution of gamma linolenic acid (6,9,12-Octadecatrienoic acid, Sigma L-2378) (stress G) or arachidonic acid (5,8,11,14-Eicosatetraenoic acid, Sigma A-3925) (stress A) (400 μM in water with 0.125% (v/v) Triton X-100) to completely cover the leaves. Twenty to twenty-four hours after the stress, plants were harvested.

Harvest Solid S1 and Optional Storage Treatment: Twenty to twenty-four hours after the stress, more than 4 grams of leaves, stems, fruit, flowers, seeds or other plant parts were harvested and frozen immediately in dry ice, then transferred as soon as possible to a −20° C. freezer until use. Plant materials may be stored at −20 C for a long period of time, more than a year, without losing inhibitory activity. Temperature was monitored to ensure a constant condition.

Stressed and non-stressed plant specimens were collected as wet samples and stored at −20° C. for various periods of time, and were submitted to a process which generates 3 subfractions: aqueous, ethanolic and organic fractions. The complete extraction process was performed in a continuous cycle using the following steps. An initial 5 g of plant specimen was homogenized in liquid nitrogen with a blender. The resulting powder was weighed.

Extraction Process 1—Aqueous Extraction: To each 4.5 grams of plant powder, 12 ml of a cold solution of 100 mM Tris, pH 7.0 was added. The mixture was thoroughly vortexed for 2 minutes. The mixture was kept on ice for 30 minutes and vortexed after each 10 minute period of time. The sample was centrifuged in a Corex™ 30 ml tube for 5 minutes at 4500 rpm. The resulting supernatant was decanted in a 15 ml tube after filtration with a Miracloth™ filter. This extract represents Potential Extract A. The pellet, referred to as Solid S2, was kept for ethanolic extraction.

The aqueous extract (Potential Extract A) was further purified in order to determine its EP inhibition capability. The Potential Extract A was purified by size-exclusion chromatography, wherein the aqueous extract was chromatographed on a calibrated Sephadex G-25 column (1×10 cm) using a 20 mM Tris-HCl, 150 mM NaCl, pH 7.5 buffer as eluant. Fractions corresponding to compounds that appeared to have a molecular weight (MW) less than 1500 daltons (D) were pooled to constitute the purified aqueous extract that was tested for inhibitory activity as described in Example II.

Prior to this analysis, the extract was treated with 10% gelatin-Sepharose (Pharmacia Biotech, Uppsala, Sw.) in order to remove unspecific enzyme ligands. To 1 mL of extract, 100 μL of gelatin-Sepharose resin was added in a microassay tube, the solution in the tube was mixed, kept on ice for 30 minutes, and then centrifuged 5 minutes at 5,000 rpm. The supernatant was removed and used directly for assays.

Extraction Process II—Alcoholic Extraction: To the pellet, Solid S2, collected from the previous aqueous extraction, 12 ml of cold ethanol:methanol (85:15) was added and the mixture was thoroughly vortexed for 2 minutes. The mixture was kept on ice for 30 minutes and vortexed every 10 minutes. The sample was centrifuged in a Corex™ 30 ml tube for 5 minutes at 4,500 rpm. The resulting supernatant was decanted in a 15 ml tube after filtration with a Miracloth™ filter. The pellet, referred to as Solid S3, was kept for the subsequent organic extraction. This extract represents Potential Extract B. The ethanolic extract, Potential Extract B, was purified by liquid/liquid extraction prior to analysis by enzymatic assay. For this purpose, 1 ml of ethanolic extract was evaporated under vacuum, dissolved in 150 μl of dimethylsulfoxide (DMSO), and completed to a final volume of 1.5 ml with Tris buffer (final concentration: Tris-HCl 20 mM; pH 7.5). Four ml of hexane was added to the Tris phase in a glass tube and the tube was thoroughly vortexed, then allowed to form a biphasic liquid. The organic phase was removed and the extract was submitted to a second round of liquid/liquid extraction. The aqueous phase was removed and treated with 10% gelatin-Sepharose (Pharmacia Biotech, Uppsala, Sw) to remove unspecific enzyme ligands prior to conducting subsequent assays. To 1 ml of extract, 100 μL of gelatin-Sepharose resin was added in a microassay tube, the tube was mixed, kept on ice for 30 minutes, and then centrifuged 5 minutes at 5,000 rpm. Supernatant was removed and used directly for assays as described in Example H.

Extraction Process III—Organic Extraction: To the pellet, Solid S3, collected from previous ethanolic extraction, 12 ml of cold dichloromethane was added and the mixture was thoroughly vortexed for 2 minutes. The mixture was kept on ice for 30 minutes and vortexed after each 10 minutes period. The sample was centrifuged in a Corex™ 30 ml tube for 5 minutes at 4,500 rpm. The resulting supernatant was decanted in a 15 ml glass tube after filtration with a Miracloth™ filter. The final pellet was discarded. The organic solvent was evaporated under vacuum and the phase was dissolved with dimethylsulfoxide (DMSO). This extract represents Potential Extract C, which was further purified by solid phase extraction prior to analysis by enzymatic assay.

In order to assay the Potential Extract C, the organic extract was diluted 1:10 in a solution of DMSO:Methanol:Tris (20 mM, pH 7.5) (10:50:40) (Solution A), i.e., 220 μl of extract was added to 2.0 ml of solution A. After 10 seconds of vigorous vortex, the mix was sonicated for 10 seconds. Dissolved extracts were subsequently applied to a solid phase extraction plate (Discovery SPE-96, Sigma Chemical Co, St-Louis, Mo.). After initial conditioning of the columns with 1 ml of methanol, columns were equilibrated with solution A, and extract samples were deposited on the columns. Elution was completed with solution A (final volume of 2 ml) and this fraction was used directly in assays as described in Example II.

### Example II

### In Vitro Enzyme Inhibition Assays

The inhibitory activity of sample compositions towards human MMP-9 or human cathepsin-B were determined using either fluorogenic substrates or the FASC assay.

Measurement of Human MMP-9 Activity with Fluorogenic Peptidic Substrates

MMP-9 was purified from natural sources (THP-1 cells (ATCC, Manassas, Va.) for MMP-9) as described in literature and based on protocols found in I. M. Clark: “Matrix metalloproteinases protocols”, Humana Press (2001). Proteolytic activity of MMP-9 was evaluated with the assay based on the cleavage of auto-quenched peptide substrate: (MCA-Pro-Leu-Gly-Leu-Dpa-Ala-Arg-NH₂.TFA [Dpa=N-3-(2,4-dinitrophenyl)-L-2,3-diaminopropionyl]); In the intact peptide, Dpa or DNP quenches the MCA fluorescence. Cleavage of the peptide causes release of the fluorescent MCA group which was then quantitated on a fluorometer (Gemini X S, Molecular Devices, Sunnyvale, Calif.). The assay was performed in TNCZ assay buffer (20 mM Tris-HCl; NaCl 150 mM; CaCL₂ 5 mM; ZnCl₂ 0.5 mM; pH 7.5) with human purified proteases (I. M. Clark: Matrix metalloproteinases protocols, Humana Press (2001)). The substrate, primarily dissolved in DMSO was then redissolved in TNCZ buffer for the assay. In a typical assay, 10 μl of purified enzyme (1-50 ng) and 5 μl of dissolved substrate (final concentration of 10 μM) was mixed in a final volume of 75 μl (completed with TNCZ). All assays were performed in 96 well plate and the reaction was started by the addition of substrate. Assays were measured (excitation 325 nm, emission 392 nm) for 20, 40 and 60 minutes.

### Measurement of Human MMP-9, Cathepsin B Activity Using the FASC Assay

Human Cathepsin B was obtained from Calbiochem (San Diego, Calif.). Human MMP-9 was purified as previously described. The assay was based on the method described in Canadian Patent No. 2,189,486 (1996) and by St-Pierre et al., (Cytometry (1996) 25:374-380. For the assay, 5 μl of the purified enzyme (1-100 ng), 5 μl of concentrated buffer solution (20 mM Tris-HCl; NaCl 150 mM; CaCL₂ 5 mM; ZnCl₂ 0.5 mM; pH 7.5), and 5 μl of gelatin-FITC beads were typically used in a final volume of 100 μl. The assay was performed by incubation of the reaction mixture for 90 minutes at 37° C. The reaction was stopped by the transfer of the mix in 0.5 ml of 20 mM Tris, 150 mM NaCl; pH 9.5 buffer. This tube was analyzed in a flow cytometer (Epics MCL, Beckman Coulter, Mississauga, Ontario) as described in Canadian Patent No. 2,189,486 (1996).

Measurement of Human Cathepsin B Activity with a Fluorogenic Proteic Substrate

Cathepsin B was obtained as previously described. The activities of Cathepsin B was measured by an assay based on the increase of fluorescence of a proteic substrate (Haemoglobin) heavily labelled with Alexa-488 dye (Molecular Probes, Eugene, Oreg.). The substrate, when highly labelled with the dye, will almost quench the dye fluorescence. Cleavage of the substrate will result in an increase of the fluorescence which can be measured with a spectrofluorometer, and which was proportional to protease activity. Typically, 10 μl of purified human Cathepsin B, and 10 μL of Hemoglobin-Alexa488 or beta-casein-Alexa488 (100 ng) were assayed in final volume of 75 μl adjusted with 20 mM citrate pH 3.3 buffer. The reaction was performed as already described except that the fluorescence was read at excitation 488 nm/emission 525 nm wavelengths.

### Extract Inhibition Assay

Before a typical assay, aqueous extracts prepared as described in Example I were preincubated with 1:10 of gelatin-Sepharose 4B™ for 30 minutes to remove fluorescence quenching. For the ethanolic extract, an initial hexane extraction was performed and samples were treated with 1:10 of gelatin-Sepharose 4B™ to remove quenching.

In a typical fluorescent assay, 10 μl of purified enzyme at concentrations previously mentioned for the enzymatic assay, 5 μl of dissolved fluorogenic peptide or 10 μl of dissolved fluorescent proteic substrate (final concentration of 10 μM) and 40 μL, of the aqueous, ethanolic or organic extract to be tested and prepared as described in Example I were mixed in a final volume of 75 μl (completed with TNCZ for fluorogenic peptide substrate assay or 20 mM citrate pH 3.3 buffer for fluorescent protein substrate assay). All assays were performed in 96 well plates and the reaction was started by the addition of substrate. Assays were measured (excitation 325 nm, emission 392 nm for peptide and excitation 488 nm/emission 525 μm wavelengths for protein) for 20, 40 and 60 minutes. Activity and inhibition values were determined from the increase in fluorescence

For the FASC assay, 35 μl of the treated extract prepared as described in Example I, 5 μl of the purified enzyme prepared as described previously, 5 μl of concentrated buffer solution (TNCZ), and 5 μl of gelatin-FITC beads were typically used. The initial step of the assay was the incubation of the reaction without beads for a 30 minutes period on ice to allow the binding of inhibitors to enzyme. Fluorescent beads were added and the reaction mix was incubated for 90 minutes at 37° C. The reaction was stopped by transfer of the mix in 0.5 ml of 20 mM Tris, 150 mM NaCl; pH 9.5 buffer. This tube was analyzed in the flow cytometer (Epics MCL, Beckman Coulter, Mississauga, Ontario) as described in Canadian Patent Application No. 2,189,486 (1996).

The results from the above assays for MMP-9 and cathepsin B are presented in Tables 6 and 7, respectively. In these tables the following abbreviations are used:

Str=Stress. In this column the following abbreviations represent the stress applied during preparation of the extract: A:Arachidonic Acid; G:Gamma-Linolenic Acid; N: No stress treatment
Extr=Extract. In this column the following abbreviations represent the solvent used to prepare the extract: S:Organic; O:Aqueous; R:Alcoholic.

TABLE 6
Plant Extracts Capable of Inhibiting MMP-9

| | | | Inhibition | | | | Inhibition |
|---|---|---|---|---|---|---|---|
| Latin name | Str | Extr | (%) | Latin name | Str | Extr | (%) |
| Abelmochus esculentus | A | S | 26.8 | | | | |
| Achillea millefolium | A | S | 41.6 | | | | |
| Aconitum napellus | A | O | 47.7 | | | | |
| Acorus calamus | A | O | 83.2 | | | | |
| Actinidia arguta | A | S | 26.8 | | | | |
| Adiantum pedatum | A | O | 20.7 | | | | |
| Agastache foeniculum | A | S | 100.0 | | | | |
| Agrimonia eupatoria | A | R | 21.4 | | | | |
| Agropyron cristatum | A | R | 51.4 | | | | |
| Agropyron repens | A | S | 27.3 | | | | |
| Agrostis alba | A | R | 40.6 | | | | |
| Agrostis stofonifera | A | R | 35.4 | | | | |
| Alcea rosea | A | S | 45.8 | | | | |
| Alkanna tinctoria | A | S | 42.5 | | | | |
| Allium cepa | A | O | 49.7 | | | | |
| Allium grande | A | R | 71.4 | Arrhenatherum elatius | A | R | 40.4 |
| Allium porrum | A | S | 28.0 | Artemisia dracunculus | A | S | 51.1 |
| Allium porrum | A | O | 82.0 | Asparagus officinalis | A | S | 20.9 |
| Allium sativum | A | S | 23.7 | Asparagus officinalis | A | S | 32.6 |
| Allium schoenoprasum | A | O | 45.5 | Aster sp | A | O | 29.5 |
| Allium tuberosum | A | O | 20.1 | Aster sp | A | R | 80.0 |
| Allium tuberosum | A | O | 91.5 | Atropa belladonna | A | S | 47.4 |
| Althaea officinalis | A | S | 29.6 | Beta vulgaris | A | S | 25.3 |
| Amaranthus gangeticus | A | O | 25.1 | Beta vulgaris | A | R | 26.6 |
| Amaranthus gangeticus | A | R | 31.1 | Beta vulgaris | A | R | 34.0 |
| Amaranthus gangeticus | A | S | 73.2 | Beta vulgaris | A | O | 42.0 |
| Amaranthus retroflexus | A | S | 20.4 | Beta vulgaris | A | O | 44.0 |
| Ambrosia artemisiifolia | A | R | 50.1 | Beta vulgaris subsp.. | A | R | 44.0 |
| | | | | Maritima | | | |
| Amelanchier sanguinea | A | R | 37.6 | Beta vulgaris var. | A | R | 35.4 |
| | | | | Condivata | | | |
| Anthemis nobilis | A | O | 40.4 | Brassica napus | A | S | 24.6 |
| Anthemis nobilis | A | R | 66.7 | Brassica napus | A | R | 53.1 |
| Anthemis tinctorium | A | S | 30.3 | Brassica napus | A | O | 100.0 |
| Apium graveolens | A | R | 71.2 | Brassica nigra | A | S | 24.2 |
| Arachis hypogaea | A | O | 23.5 | Brassica oleracea | A | R | 33.0 |
| Aralia cordata | A | S | 21.2 | Brassica oleracea | A | R | 36.0 |
| Aralia cordata | A | S | 56.3 | Brassica oleracea | A | R | 36.2 |
| Arctium minus | A | R | 31.1 | Brassica oleracea | A | S | 73.1 |
| Arctostaphylos uva-ursi | A | S | 31.2 | Brassica oleracea | A | O | 100.0 |
| Arctostaphylos uva-ursi | A | O | 31.2 | Brassica rapa | A | R | 31.0 |
| Arctostaphylos uva-ursi | A | R | 59.7 | Brassica rapa | A | R | 38.6 |
| Armoracia rusticana | A | R | 25.1 | Brassica rapa | A | O | 42.8 |
| Armoracia rusticana | A | S | 56.2 | Brassica rapa | A | R | 48.8 |
| Aronia melanocarpa | A | S | 26.8 | Brassica rapa | A | S | 68.2 |
| Aronia melanocarpa | A | S | 41.3 | Brassica rapa | A | O | 89.2 |
| Aronia melanocarpa | A | O | 44.8 | Bromus inermis | A | R | 51.4 |
| Aronia melanocarpa | A | R | 47.7 | Campanula rapunculus | A | O | 25.1 |
| Aronia melanocarpa | A | R | 55.7 | Canna edulis | A | S | 31.1 |
| Aronia melanocarpa | A | O | 100.0 | Canna edulis | A | O | 47.6 |
| Canna edulis | A | R | 68.9 | Cosmos sulphureus | A | O | 37.0 |
| Capsella bursa-pastoris | A | R | 32.5 | Crataegus sp | A | O | 32.4 |
| Capsicum annuum | A | O | 22.0 | Crataegus sp | A | S | 45.5 |
| Capsicum annuum | A | R | 24.0 | Crataegus sp | A | R | 100.0 |
| capsicum annuum | A | S | 55.7 | Crataegus submollis | A | S | 45.5 |
| Capsicum frutescens | A | S | 30.3 | Cryptotaenia canadensis | A | R | 26.4 |
| Capsicum frutescens | A | O | 34.7 | Cucumis anguria | A | R | 27.2 |
| Carthamus tinctorius | A | R | 28.5 | Cucumis anguria | A | S | 36.6 |
| Carum carvi | A | S | 38.6 | Cucumis anguria | A | O | 38.5 |
| Chelidonium majus | A | O | 27.9 | Cucumis melo | A | O | 59.2 |
| Chenopodium bonus- | A | R | 47.4 | Cucumis sativus | A | R | 39.8 |
| henricus | | | | | | | |
| Chenopodium bonus- | A | O | 20.7 | Cucumis sativus | A | O | 49.4 |
| henricus | | | | | | | |
| Chenopodium bonus- | A | R | 23.2 | Cucumis sativus | A | S | 54.4 |
| henricus | | | | | | | |
| chenopodium bonus- | A | S | 62.8 | Cucurbita maxima | A | O | 46.7 |
| henricus | | | | | | | |
| Chenopodium quinoa | A | O | 23.1 | Cucurbita moschata | A | S | 32.1 |
| Chenopodium quinoa | A | R | 34.7 | Cucurbita pepo | A | O | 37.0 |
| Chrysanthemum | A | O | 20.6 | Curburbita pepo | A | R | 41.0 |
| leucanthemum | | | | | | | |
| Chrysanthemum | A | R | 30.9 | Curburbita pepo | A | S | 43.9 |
| leucanthemum | | | | | | | |
| Chrysanthemum | A | R | 26.4 | Curcuma zedoaria | A | S | 67.6 |
| coronarium | | | | | | | |
| var. spatiosum | | | | | | | |
| Chrysanthenum | A | S | 66.6 | Curcurbita maxima | A | S | 25.8 |
| coronarium | | | | | | | |
| Cichorium intybus | A | S | 44.7 | Cymbopogon citratus | A | O | 26.7 |
| Citrullus lanatus | A | S | 62.1 | Dactylis glomerata | A | R | 27.2 |
| Citrullus lanatus | A | O | 70.6 | Datisca cannabina | A | S | 26.9 |
| Cornus canadensis | A | S | 48.5 | Datisca cannabina | A | O | 38.0 |
| Cosmos sulphureus | A | S | 23.4 | Daucus carota | A | R | 30.8 |
| Daucus carota | A | O | 31.9 | Hamamelis virginiana | A | S | 41.0 |
| Dirca palustris | A | O | 27.3 | Hamamelis virginiana | A | R | 74.6 |
| Dirca palustris | A | S | 34.2 | Hedeoma pulegioides | A | O | 22.0 |
| Dolicos lablab | A | S | 22.0 | Helianthus tuberosus | A | R | 21.2 |
| Dolicos lablab | A | R | 25.3 | Helianthus tuberosus | A | R | 51.5 |
| Dryopteris filix-mas | A | S | 24.9 | Helichrysum | A | O | 21.0 |
| | | | | Angustifolium | | | |
| Dryopteris filix-mas | A | R | 40.6 | Heliotropium | A | S | 54.1 |
| | | | | Arborescens | | | |
| Eleusine coracana | A | S | 20.2 | Helleborus niger | A | S | 37.8 |
| Eleusine coracana | A | R | 20.9 | Hordeum hexastichon | A | R | 38.0 |
| Eleusine coracana | A | O | 71.1 | Hyssopus officinalis | A | O | 25.1 |
| Elymus junceus | A | R | 45.4 | Inula helenium | A | S | 29.7 |
| Erigeron canadensis | A | S | 35.7 | Isatis tinctoria | A | S | 41.5 |
| Eruca vesicaria | A | R | 59.9 | Lactuca serriola | A | R | 41.3 |
| Fagopyrum esculentum | A | O | 20.7 | Lactuca serriola | A | S | 46.6 |
| Fagopyrum tartaricum | A | R | 30.3 | Laportea canadensis | A | S | 26.3 |
| Fagopyrum tartaricum | A | O | 33.2 | Lathyrus sativus | A | O | 22.2 |
| Festuca rubra | A | R | 31.8 | Lathyrus sativus | A | R | 50.2 |
| Foeniculum vulgare | A | R | 27.4 | Lathyrus sylvestris | A | O | 31.3 |
| Foeniculum vulgare | A | O | 50.6 | Lathyrus sylvestris | A | R | 31.8 |
| Forsythia x intermedia | A | O | 100.0 | Laurus nobilis | A | S | 25.7 |
| Fragaria x ananassa | A | O | 30.0 | Laurus nobilis | A | O | 30.0 |
| Fragaria x ananassa | A | S | 36.3 | Lavandula latifolia | A | S | 40.3 |
| Galium odoratum | A | R | 26.9 | Leonurus cardiaca | A | R | 27.0 |
| Gaultheria hispidula | A | R | 28.4 | Lepidium sativum | A | S | 41.8 |
| Gaultheria hispidula | A | S | 40.7 | Levisticum officinale | A | S | 29.0 |
| Gentiana lutea | A | R | 34.7 | Levisticum officinale | A | O | 44.9 |
| Glechoma hederacea | A | S | 37.6 | Linaria vulgaris Miller | A | O | 23.6 |
| Glycine max | A | R | 38.1 | Linum usitatissimum | A | R | 33.3 |
| Glycine max | A | O | 56.4 | Lolium multiflorum | A | S | 29.0 |
| Glycine max | A | S | 71.4 | Lolium perenne | A | R | 52.0 |
| Glycyrrhiza glabra | A | S | 62.6 | Lotus corniculatus | A | R | 62.9 |
| Glycyrrhiza glabra | A | R | 100.0 | Lotus tetragonolobus | A | S | 62.9 |
| Guizotia abyssinica | A | R | 91.9 | Lycopersicon esculentum | A | S | 26.1 |
| Lycopersicon esculentum | A | R | 33.0 | Pastinaca sativa | A | R | 46.9 |
| Malva moschata | A | S | 31.8 | Phalaris canariensis | A | R | 20.3 |
| Malva sylvestris | A | S | 21.4 | Phalaris canariensis | A | O | 80.5 |
| Malva verticillata | A | R | 43.4 | Phaseolus mungo | A | O | 51.3 |
| Matteucia pensylvanica | A | R | 26.9 | Phaseolus mungo | A | S | 74.1 |
| Medicago sativa | A | O | 20.4 | Phaseolus vulgaris | A | O | 23.0 |
| Melilotus albus | A | R | 53.9 | Phaseolus vulgaris | A | O | 51.4 |
| Melissa officinalis | A | S | 21.4 | Phaseolus vulgaris | A | S | 62.6 |
| Melissa officinalis | A | O | 36.8 | Phlox paniculata | A | O | 41.0 |
| Melissa officinalis | A | R | 53.7 | Physalis alkekengi | A | R | 31.6 |
| Mentha piperita | A | S | 57.7 | Physalis ixocarpa | A | S | 45.2 |
| Mentha pulegium | A | S | 66.1 | Physalis ixocarpa | A | O | 65.3 |
| Mentha spicata | A | S | 67.7 | Physalis pruinosa | A | O | 87.3 |
| Mentha suaveolens | A | S | 51.8 | Phytolacca americana | A | S | 49.6 |
| Momordica charantia | A | R | 29.7 | Phytolacca americana | A | O | 89.8 |
| Momordica charantia | A | S | 72.1 | Pimpinella anisum | A | S | 100.0 |
| Nicotiana rustica | A | O | 30.3 | Plantago coronopus | A | S | 48.3 |
| Nicotiana rustica | A | S | 59.1 | Plantago coronopus | A | O | 89.3 |
| Nicotiana tabacum | A | S | 39.0 | Plantago major | A | S | 21.8 |
| Nicotiana tabacum | A | R | 47.6 | Poa compressa | A | R | 22.4 |
| Nicotiana tabacum | A | O | 100.0 | Poa compressa | A | S | 49.3 |
| Nigella sativa | A | R | 59.4 | Poa pratensis | A | R | 22.4 |
| Oenothera biennis | A | O | 21.3 | Polygonum | A | S | 43.3 |
| | | | | pensylvanicum | | | |
| Oenothera biennis | A | O | 36.7 | Polygonum persicaria | A | O | 21.6 |
| Origanum vulgare | A | R | 21.3 | Polygonum persicaria | A | S | 38.5 |
| Origanum vulgare | A | O | 42.7 | Potentilla anserina | A | S | 26.3 |
| Oryza sativa | A | R | 56.5 | Potentilla anserina | A | O | 31.2 |
| Oxyria digyna | A | R | 35.1 | Poterium sanguisorba | A | S | 29.2 |
| Oxyria digyna | A | O | 76.4 | Pteridium aquilinum | A | S | 27.3 |
| Pastinaca sativa | A | O | 20.3 | Raphanus sativus | A | R | 22.7 |
| Pastinaca sativa | A | R | 23.2 | Raphanus sativus | A | R | 30.8 |
| Pastinaca sativa | A | O | 42.1 | Raphanus sativus | A | R | 40.2 |
| Raphanus sativus | A | S | 71.5 | Rumex acotosa | A | R | 25.5 |
| Raphanus sativus | A | O | 100.0 | Rumex crispus | A | R | 73.3 |
| Rheum rhabarbarum | A | S | 21.3 | Rumex acetosa | A | R | 25.5 |
| Rheum rhabarbarum | A | O | 67.9 | Rumex crispus | A | R | 73.3 |
| Rheum rhabarbarum | A | R | 72.4 | Rumex crispus | A | O | 60.5 |
| Ribes nidigrolaria | A | R | 32.6 | Rumex patientia | A | O | 49.4 |
| Ribes nidigrolaria | A | O | 64.6 | Rumex patientia | A | S | 65.8 |
| Ribes nigrum | A | R | 23.6 | Rumex scutatus | A | R | 25.5 |
| Ribes nigrum | A | O | 27.2 | Rumex scutatus | A | O | 61.9 |
| Ribes nigrum | A | S | 41.0 | Rumex scutatus | A | O | 93.8 |
| Ribes nigrum | A | O | 65.8 | Ruta graveolens | A | S | 25.8 |
| Ribes nigrum | A | R | 100.0 | Ruta graveolens | A | R | 27.1 |
| Ribes sativum | A | R | 75.4 | Salix purpurea | A | S | 22.1 |
| Ribes sylvestre | A | O | 27.7 | Salix purpurea | A | R | 33.8 |
| Ribes sylvestre | A | R | 100.0 | Salvia elegans | A | R | 23.7 |
| ribes uva-crispa | A | S | 24.4 | Salvia officinalis | A | O | 20.8 |
| Ribes uva-crispa | A | R | 36.6 | Salvia officinalis | A | S | 31.4 |
| Ricinus communis | A | R | 21.6 | Salvia sclarea | A | S | 28.0 |
| Rosa rugosa | A | O | 30.6 | Satureja montana | A | R | 21.7 |
| Rosa rugosa | A | S | 36.2 | Scuttellaria lateriflora | A | S | 54.1 |
| Rosa rugosa | A | R | 39.3 | Secale cereale | A | O | 22.6 |
| Rosmarinus officinalis | A | R | 27.2 | Secale cereale | A | S | 22.9 |
| Rosmarinus officinalis | A | R | 45.7 | Secale cereale | A | R | 26.9 |
| Rubus allegheniensis | A | S | 53.7 | Sesamum indicum | A | O | 21.2 |
| Rubus canadensis | A | O | 27.0 | Setaria italica | A | O | 27.0 |
| Rubus canadensis | A | S | 41.0 | Sium sisarum | A | R | 32.6 |
| Rubus canadensis | A | R | 41.2 | Sium sisarum | A | O | 42.7 |
| Rubus canadensis | A | S | 45.1 | Solanum dulcamara | A | S | 43.3 |
| Rubus idaeus | A | O | 24.3 | Solanum dulcamara | A | O | 48.6 |
| Rubus idaeus | A | S | 39.7 | Solanum melanocerasum | A | O | 21.3 |
| Rubus idaeus | A | R | 62.2 | Solanum melongena | A | R | 20.5 |
| Rubus idaeus | A | R | 37.0 | Solanum melongena | A | O | 35.6 |
| Rumex acetosella | A | O | 75.8 | Solanum melongena | A | O | 49.4 |
| Spinacia oleracea | A | S | 41.0 | Solanum melongena | A | S | 65.2 |
| Stachys affinis | A | R | 22.5 | Solidago sp | A | R | 32.7 |
| Stachys affinis | A | S | 43.9 | Vaccinum macrocarpon | A | S | 100.0 |
| Stachys affinis | A | O | 92.0 | Veratrum viride | A | S | 29.1 |
| Symphytum officinale | A | S | 28.0 | Veratrum viride | A | O | 31.8 |
| Tanacetum | A | O | 20.3 | Verbascum thapsus | A | S | 42.6 |
| cinerariifolium | | | | | | | |
| Tanacetum | A | R | 69.7 | Verbascum thapsus | A | O | 75.2 |
| cinerariifolium | | | | | | | |
| Tanacetum vulgare | A | O | 20.2 | Viburnum trilobum | A | O | 97.4 |
| Tanacetum vulgare | A | S | 84.2 | Vicia sativa | A | R | 53.3 |
| Teucrium chamaedrys | A | O | 20.4 | Vicia villosa | A | R | 48.9 |
| Teucrium chamaedrys | A | R | 20.4 | Vigna unguiculata | A | R | 27.0 |
| Thymus serpyllum | A | R | 24.3 | Vigna unguiculata | A | O | 44.8 |
| Thymus vulgaris | A | S | 42.5 | Vigna unguiculata | A | S | 55.5 |
| Thymus x citriodorus | A | R | 27.4 | Vinca minor | A | S | 35.1 |
| Tragopogon porrifolius | A | R | 21.9 | Vitis sp. | A | O | 52.2 |
| Tragopogon porrifolius | A | O | 26.2 | Vitis sp. | A | S | 59.6 |
| Trifolium hybridum | A | R | 30.9 | Vitis sp. | A | R | 87.8 |
| Trifolium pannonicum | A | R | 41.0 | Xanthium sibiricum | A | S | 57.1 |
| Trifolium repens | A | R | 51.3 | Zea mays | A | O | 26.1 |
| Trigonella foenum- | A | S | 44.2 | Zea mays | A | R | 32.1 |
| graecum | | | | | | | |
| Triticum spelta | A | S | 30.0 | Zea mays | A | O | 38.7 |
| Triticum turgidum | A | S | 31.3 | Achillea millefolium | G | S | 45.5 |
| Typha latifolia | A | S | 57.7 | Aconitum napellus | G | S | 24.0 |
| Urtica dioica | A | O | 26.5 | Aconitum napellus | G | O | 53.9 |
| Urtica dioica | A | S | 50.2 | Acorus calamus | G | O | 87.6 |
| Vaccinium corymbosum | A | R | 39.9 | Acorus calamus | G | S | 100.0 |
| Vaccinium corymbosum | A | S | 64.8 | Actinidia arguta | G | S | 33.8 |
| Vaccinum augustifolium | A | R | 44.8 | Adiantum pedatum | G | R | 31.6 |
| | | | | Adiantum pedatum | G | S | 31.7 |
| | | | | Ageratum conyzoides | G | S | 23.1 |
| Agropyron cristatum | G | R | 64.1 | Armoracia rusticana | G | S | 62.7 |
| Agropyron repens | G | S | 29.2 | Aronia melanocarpa | G | O | 26.7 |
| Agropyron repens | G | O | 32.6 | Aronia melanocarpa | G | O | 100.0 |
| Agrostis stolonifera | G | R | 34.4 | Aronia melanocarpa | G | R | 100.0 |
| Alcea rosea | G | S | 22.7 | Aronia melanocarpa | G | R | 39.1 |
| | | | | (Michx.) Ell. | | | |
| Alchemilla mollis | G | S | 30.5 | Artemisia dracunculus | G | O | 44.3 |
| Alchemilla mollis | G | R | 33.2 | Artemisia dracunculus | G | S | 65.4 |
| Allium ampeloprasum | G | O | 53.4 | Asclepias incarnata | G | R | 20.3 |
| Allium cepa | G | S | 22.5 | Asparagus officinalis | G | O | 22.3 |
| Allium cepa | G | O | 60.7 | Asparagus officinalis | G | S | 26.6 |
| Allium schoenoprasum | G | S | 21.1 | Asparagus officinalis | G | R | 28.7 |
| Allium schoenoprasum | G | O | 60.4 | Aster sp | G | O | 34.3 |
| Allium tuberosum | G | S | 38.8 | Aster sp | G | R | 62.6 |
| Allium tuberosum | G | O | 74.4 | Atropa belladonna | G | S | 34.9 |
| Althaea officinalis | G | S | 54.9 | Beta vulgaris | G | R | 28.3 |
| Amaranthus candathus | G | O | 42.6 | Beta vulgaris | G | R | 42.2 |
| Amaranthus caudathus | G | R | 27.1 | Beta vulgaris | G | O | 47.0 |
| Amaranthus gangeticus | G | S | 56.8 | Beta vulgaris spp. | G | O | 46.7 |
| | | | | Maritima | | | |
| Amaranthus gangeticus | G | S | 74.4 | Brassica cepticepa | G | R | 26.7 |
| Ambrosia artemisiifolia | G | R | 49.0 | Brassica cepticepa | G | S | 68.3 |
| Amelanchier sanguinea | G | R | 45.2 | Brassica juncea | G | O | 45.0 |
| Angelica archangelica | G | S | 20.9 | Brassica juncea | G | S | 66.1 |
| Anthemis nobilis | G | R | 58.9 | Brassica napus | G | S | 27.5 |
| Apium graveolens | G | O | 30.4 | Brassica napus | G | R | 37.6 |
| Apium graveolens | G | S | 36.4 | Brassica napus | G | O | 94.8 |
| Apium graveolens | G | R | 60.6 | Brassica nigra | G | S | 36.4 |
| Arachis hypogaea | G | R | 26.0 | Brassica oleracea | G | R | 38.7 |
| Aralia cordata | G | S | 66.0 | Brassica oleracea | G | R | 39.0 |
| Arctium minus | G | O | 26.6 | Brassica oleracea | G | R | 49.4 |
| Arctium minus | G | R | 30.8 | Brassica oleracea | G | S | 76.1 |
| Arctostaphylos uva-ursi | G | S | 29.3 | Brassica oleracea | G | O | 100.0 |
| Arctostaphylos uva-ursi | G | O | 38.8 | Brassica rapa | G | R | 21.1 |
| Arctostaphylos uva-ursi | G | R | 80.2 | Brassica rapa | G | S | 64.0 |
| Brassica rapa | G | O | 100.0 | Coix Lacryma-Jobi | G | O | 21.0 |
| Bromus inermis | G | R | 36.7 | Cornus canadensis | G | S | 34.8 |
| Campanula rapunculus | G | O | 59.9 | Crataegus sp | G | R | 54.0 |
| Canna edulis | G | O | 20.8 | Crataegus submollis | G | S | 31.3 |
| Canna edulis | G | O | 83.1 | Cryptotaenia canadensis | G | R | 32.1 |
| Capsicum annuum | G | R | 20.2 | Cucumis anguria | G | S | 27.3 |
| Capsicum annuum | G | S | 29.6 | Cucumis anguria | G | O | 32.5 |
| Capsicum annuum | G | O | 51.5 | Cucumis sativus | G | O | 39.4 |
| Capsicum annuum | G | S | 60.8 | Cucumis sativus | G | S | 69.4 |
| Capsicum frutescens | G | S | 32.8 | Cucurbita maxima | G | O | 34.1 |
| Carthamus tinctorius | G | R | 29.8 | Cucurbita maxima | G | S | 42.6 |
| Carum carvi | G | S | 30.4 | Cucurbita moschata | G | S | 32.0 |
| Chelidonium majus | G | O | 39.9 | Cucurbita moschata | G | O | 39.2 |
| Chenopodium bonus- | G | O | 63.0 | Cucurbita pepo | G | S | 28.8 |
| henricus | | | | | | | |
| Chenopodium quinoa | G | O | 34.1 | Cucurbita pepo | G | O | 32.6 |
| Chenopodium quinoa | G | R | 42.8 | Curcuma zedoaria | G | O | 23.3 |
| Chenopodium quinoa | G | O | 46.1 | Curcuma zedoaria | G | S | 57.6 |
| Chichorium endivia | G | R | 22.0 | Cymbopogon citratus | G | O | 70.1 |
| subsp. endivia | | | | | | | |
| Chichorium endivia | G | S | 22.9 | Cynara scolymus | G | S | 20.2 |
| subsp. endivia | | | | | | | |
| Chrysanthemum | G | R | 23.2 | Cynara scolymus | G | O | 37.5 |
| coronarium | | | | | | | |
| Chrysanthemum | G | S | 68.4 | Cynara scolymus | G | R | 88.7 |
| coronarium | | | | | | | |
| Chrysanthemum | G | R | 20.5 | Cyperus esculentus | G | S | 66.7 |
| leucanthemum | | | | | | | |
| Cicer arietinum | G | S | 25.7 | Datura metel | G | S | 29.2 |
| Cichorium intybus | G | R | 51.1 | Datura stramonium | G | O | 27.6 |
| Cichorium intybus | G | S | 53.4 | Daucus carota | G | O | 24.2 |
| Citrullus lanatus | G | S | 36.5 | Daucus carota | G | R | 29.3 |
| Citrullus lanatus | G | O | 71.5 | Dipsacus sativus | G | S | 48.7 |
| Coix lacryma-jobi | G | O | 21.0 | | | | |
| Dirca palustris | G | O | 29.9 | Glycyrrhiza glabra | G | R | 100.0 |
| Dirca palustris | G | S | 36.4 | Guizotia abyssinica | G | R | 91.4 |
| Dolichos lablab | G | S | 35.8 | Hamamelis virginiana | G | O | 39.8 |
| Dolichos lablab | G | R | 74.5 | Hamamelis virginiana | G | R | 78.8 |
| Dryopteris filix-mas | G | S | 27.9 | Hamamelis virginiana | G | S | 96.6 |
| Dryopteris filix-mas | G | R | 42.6 | Hedeoma pulegioides | G | S | 45.4 |
| Echinochloa frumentacea | G | O | 68.4 | Helenium hoopesii | G | S | 22.6 |
| Eleusine coracana | G | O | 47.8 | Helenium hoopesii | G | O | 52.8 |
| Elymus junceus | G | R | 42.7 | Helianthus annuus | G | R | 22.0 |
| Erigeron canadensis | G | S | 37.8 | Helianthus annuus | G | S | 31.6 |
| Erigeron speciosus | G | R | 34.6 | Helianthus strumosus | G | R | 30.5 |
| Errhenatherum elatius | G | R | 34.4 | Helianthus strumosus | G | O | 71.7 |
| Fagopyrum tartaricum | G | R | 31.4 | Helianthus tuberosus | G | R | 21.2 |
| Foeniculum vulgare | G | R | 28.0 | Helianthus tuberosus | G | S | 50.7 |
| Foeniculum vulgare | G | S | 44.6 | Helianthus tuberosus L. | G | R | 24.9 |
| Foeniculum vulgare | G | O | 68.9 | Heliotropium | G | S | 40.0 |
| | | | | Arborescens | | | |
| Foeniculum vulgare | G | R | 100.0 | Heliotropium | G | O | 45.6 |
| | | | | Arborescens | | | |
| Forsythia x intermedia | G | O | 100.0 | Helleborus niger | G | S | 38.0 |
| Forsythia x intermedia | G | O | 79.5 | Hordeum vulgare | G | S | 21.5 |
| Galium odoratum | G | S | 32.4 | Humulus lupulus | G | O | 35.1 |
| Galium odoratum | G | R | 100.0 | Hypericum sp. | G | R | 26.1 |
| Gaultheria hispidula | G | R | 48.4 | Hyssopus officinalis | G | S | 74.5 |
| Gaultheria hispidula | G | S | 80.4 | Iberis amara | G | O | 20.9 |
| Gaultheria hispidula | G | O | 100.0 | Iberis amara | G | S | 21.7 |
| Gaultheria procumbens | G | S | 26.9 | Inula helenium | G | S | 27.6 |
| Gaultheria procumbens | G | R | 54.3 | Ipomoea batatas | G | S | 37.5 |
| Glechoma hederacea | G | S | 26.6 | Isatis tinctoria | G | S | 48.0 |
| Glycine max | G | R | 52.5 | Lactuca serriola | G | R | 53.0 |
| Glycine max | G | O | 67.9 | Lactuca sativa | G | R | 24.5 |
| Glycine max | G | O | 75.8 | Laportea canadensis | G | S | 36.0 |
| Glycyrrhiza glabra | G | R | 21.4 | Laportea canadensis | G | O | 81.7 |
| Glycyrrhiza glabra | G | O | 21.6 | Lathyrus sativus | G | R | 37.8 |
| Lathyrus sylvestris | G | R | 40.7 | Oenothera biennis | G | O | 48.0 |
| Lathyrus sylvestris | G | O | 79.1 | Oenothera biennis | G | R | 76.6 |
| Laurus nobilis | G | S | 22.7 | Origanum vulgare | G | O | 41.3 |
| Lavandula angustifolia | G | S | 31.7 | Oryza sativa | G | O | 22.1 |
| Lavandula latifolia | G | O | 27.2 | Oxyria digyna | G | O | 26.5 |
| Ledum groenlandicum | G | S | 61.1 | Oxyria digyna | G | O | 70.3 |
| Leonurus cardiaca | G | O | 22.6 | Panicum miliaceum | G | O | 94.4 |
| Lepidium sativum | G | S | 23.3 | Pastinaca sativa | G | R | 29.4 |
| Levisticum officinale | G | S | 23.1 | Pastinaca sativa | G | S | 79.2 |
| Levisticum officinale | G | R | 27.5 | Pennisetum | G | O | 22.0 |
| | | | | alopecuroides | | | |
| Levisticum officinale | G | O | 41.3 | Petasites japonicus | G | S | 29.2 |
| Linum usitatissimum | G | R | 21.4 | Peucedanum oreaselinum | G | O | 21.3 |
| Lolium perenne | G | R | 32.7 | Phacelia tanacetifolia | G | R | 23.5 |
| Lotus corniculatus | G | R | 54.2 | Phalaris arundinacea | G | R | 47.5 |
| Malus hupehensis | G | R | 26.4 | Phalaris canariensis | G | R | 23.1 |
| Malva verticillata | G | R | 37.9 | Phalaris canariensis | G | O | 100.0 |
| Matricaria recutita | G | O | 50.3 | Phaseolus coccineus | G | O | 37.0 |
| Medicago sativa | G | R | 29.1 | Phaseolus coccineus | G | R | 74.1 |
| Melilotus albus | G | R | 52.1 | Phaseolus mungo | G | O | 42.2 |
| Melissa officinalis | G | O | 22.7 | Phaseolus mungo | G | S | 52.2 |
| Melissa officinalis | G | S | 35.9 | Phaseolus vulgaris | G | O | 35.5 |
| Melissa officinalis | G | R | 38.6 | Phaseolus vulgaris | G | S | 48.0 |
| Mentha piperita | G | S | 64.4 | Phaseolus vulgaris | G | O | 58.1 |
| Mentha suaveolens | G | R | 22.5 | Phlox paniculata | G | S | 32.2 |
| Momordica charantia | G | R | 29.3 | Phlox paniculata | G | O | 40.1 |
| Momordica charantia | G | S | 90.6 | Physalis ixocarpa | G | O | 20.6 |
| Nepeta cataria | G | R | 50.5 | Physalis pruinosa | G | O | 80.0 |
| Nicotiana rustica | G | O | 35.3 | Phytolacca americana | G | S | 62.0 |
| Nicotiana rustica | G | S | 100.0 | Phytolacca americana | G | O | 100.0 |
| Nicotiana tabacum | G | S | 31.6 | Pimpinella anisum | G | S | 37.3 |
| Nicotiana tabacum | G | O | 100.0 | Pisum sativum | G | R | 34.4 |
| Nigella sativa | G | R | 24.2 | Pisum sativum | G | O | 63.3 |
| Ocimum basilicum | G | S | 30.6 | Plantago coronopus | G | O | 42.7 |
| Plantago coronopus | G | S | 46.4 | Rosmarinus officinalis | G | R | 60.3 |
| Plantago major | G | O | 28.3 | Rubus idaeus | G | O | 32.5 |
| Plantago major | G | S | 41.4 | Rubus idaeus | G | S | 47.0 |
| Plectranthus sp. | G | S | 29.3 | Rubus occidentalis | G | S | 39.4 |
| Poa compressa | G | R | 22.1 | Rubus occidentalis | G | R | 74.1 |
| Poa compressa | G | S | 45.5 | Rumex acetosa | G | R | 45.6 |
| Poa pratensis | G | R | 35.7 | Rumex acetosella | G | R | 22.8 |
| Polygonum | G | S | 38.3 | Rumex acetosella | G | O | 31.5 |
| pensylvanicum | | | | | | | |
| Polygonum persicaria | G | S | 31.0 | Rumex crispus | G | O | 25.9 |
| Potentilla anserina | G | O | 46.8 | Rumex crispus | G | R | 70.3 |
| Poterium sanquisorba | G | S | 24.7 | Rumex patientia | G | O | 39.8 |
| Poterium sanquisorba | G | R | 30.6 | Rumex patientia | G | S | 54.2 |
| Prunus cerasifera | G | R | 45.9 | Rumex scutatus | G | R | 23.8 |
| Pteridium aquilinum | G | S | 22.4 | Rumex scutatus | G | O | 69.9 |
| Raphanus raphanistrum | G | S | 36.5 | Rumex scutatus | G | O | 78.8 |
| Raphanus raphanistrum | G | O | 75.0 | Ruta graveolens | G | R | 30.7 |
| Raphanus sativus | G | R | 20.8 | Ruta graveolens | G | S | 61.5 |
| Raphanus sativus | G | R | 27.5 | Salvia elegans | G | R | 25.4 |
| Raphanus sativus | G | S | 35.4 | Salvia elegans | G | S | 31.1 |
| Rheum rhabarbarum | G | S | 27.0 | Sambucus canadensis | G | R | 80.6 |
| Ribes grossularia | G | R | 33.7 | Sambucus ebulus | G | R | 26.1 |
| Ribes nidigrolaria | G | S | 30.7 | Sambucus ebulus | G | O | 34.4 |
| Ribes nidigrolaria | G | O | 40.5 | Sambucus ebulus | G | S | 37.8 |
| Ribes nigrum | G | O | 35.9 | Sanguisorba officinalis | G | R | 100.0 |
| Ribes nigrum | G | R | 58.6 | Santolina | G | R | 21.7 |
| | | | | chamaecyparissus | | | |
| Ribes silvestris | G | O | 26.9 | Santolina | G | S | 25.2 |
| | | | | chamaecyparissus | | | |
| Ribes silvestris | G | R | 100.0 | Satureja montana | G | O | 21.2 |
| Ricinus communis | G | R | 21.8 | Scuttellaria lateriflora | G | S | 37.0 |
| Rosmarinus officinalis | G | S | 24.7 | Secale cereale | G | S | 26.7 |
| Rosmarinus officinalis | G | R | 30.9 | Secale cereale | G | R | 27.3 |
| Serratula tinctoria | G | S | 36.2 | Tanacetum | G | R | 52.4 |
| | | | | cinerariifolium | | | |
| Serratula tinctoria | G | O | 70.3 | Tanacetum vulgare | G | R | 27.1 |
| Sesamum indicum | G | O | 27.6 | Tanacetum vulgare | G | S | 72.7 |
| Sesamum indicum | G | S | 44.3 | Teucrium chamaedrys | G | R | 24.6 |
| Silybum marianum | G | S | 34.7 | Teucrium chamaedrys | G | O | 52.8 |
| Sium sisarum | G | O | 79.0 | Thymus fragantissumus | G | R | 100.0 |
| Solanum dulcamara | G | R | 25.2 | Thymus vulgaris | G | O | 24.2 |
| Solanum dulcamara | G | S | 64.6 | Thymus x citriodorus | G | S | 23.7 |
| Solanum melongena | G | S | 36.6 | Tiarella cordifolia | G | S | 20.8 |
| Solanum melongena | G | O | 40.1 | Tiarella cordifolia | G | O | 30.8 |
| Solanum melongena | G | O | 50.0 | Tragopogon porrifolius | G | O | 22.8 |
| Solanum melongena | G | S | 74.9 | Trifolium hybridum | G | R | 24.7 |
| Solanum tuberosum | G | S | 39.1 | Trifolium pannonicum | G | R | 65.5 |
| Solanum tuberosum | G | O | 39.2 | Trifolium repens | G | R | 57.5 |
| Solidago sp. | G | R | 30.7 | Trigonella | G | S | 37.6 |
| | | | | foenumgraecum | | | |
| Sorghum caffrorum | G | O | 87.9 | Triticum furgidum | G | S | 56.5 |
| Sorghum dochna | G | R | 20.6 | Triticum spelta | G | S | 40.8 |
| Sorghum dochna | G | O | 20.6 | Tropaeolum majus | G | O | 76.1 |
| Sorghum dochna | G | S | 34.1 | Typha latifolia | G | S | 43.3 |
| Sorghum dochna | G | O | 97.0 | Urtica dioica | G | S | 40.3 |
| Sorghum durra | G | O | 30.6 | Vaccinium angustifolium | G | S | 42.4 |
| Sorghum durra | G | S | 30.6 | Vaccinium corymbosum | G | S | 61.5 |
| Sorghum durra | G | O | 48.0 | Vaccinium macrocarpon | G | S | 43.7 |
| Sorghum sudanense | G | S | 21.7 | Vaccinum angustifolium | G | R | 23.1 |
| Sorghum sudanense | G | O | 24.6 | Veratrum viride | G | S | 43.6 |
| Sorghum sudanense | G | O | 32.1 | Verbascum thapsus | G | S | 37.8 |
| Spinacia oleracea | G | S | 53.2 | Verbascum thapsus | G | O | 87.0 |
| Stachys affinis | G | S | 25.0 | Veronica officinalis | G | S | 30.5 |
| Stachys affinis | G | R | 27.8 | Viburnum trilobum | G | S | 49.4 |
| Stachys affinis | G | O | 100.0 | Viburnum trilobum | G | R | 100.0 |
| Symphytum officinale | G | R | 21.7 | Viburnum trilobum | G | O | 100.0 |
| Symphytum officinale | G | O | 25.2 | Vicia faba | G | R | 50.5 |
| Symphytum officinale | G | S | 34.6 | Vicia sativa | G | R | 42.4 |
| Vicia villosa | G | R | 89.2 | Agaricus bisporus | N | R | 44.0 |
| Vigna angularia | G | R | 28.1 | Agaricus bisporus | N | S | 46.0 |
| Vigna angularia | G | S | 71.5 | Agastache foeniculum | N | S | 70.0 |
| Vigna unguiculata | G | R | 21.0 | Ageratum conyzoides | N | S | 31.7 |
| Vigna unguiculata | G | O | 38.7 | Agropyron cristatum | N | R | 86.9 |
| Vigna unguiculata | G | S | 61.1 | Agropyron repens | N | O | 49.6 |
| Vinca minor | G | O | 33.6 | Agrostis alba | N | R | 21.9 |
| Vinca minor | G | S | 34.3 | Agrostis stolonifera | N | R | 35.8 |
| Vitis sp. | G | O | 29.0 | Alcea rosea | N | S | 35.2 |
| Vitis sp. | G | R | 50.2 | Alchemilla mollis | N | S | 37.9 |
| Vitis sp. | G | S | 53.3 | Allium ampeloprasum | N | O | 48.0 |
| Vitis sp. | G | O | 63.0 | Allium ascalonicum | N | S | 26.2 |
| Vitis sp. | G | R | 86.6 | Allium ascalonicum | N | O | 77.2 |
| Withania somnifera | G | S | 20.3 | Allium cepa | N | O | 92.6 |
| Xanthium sibiricum | G | S | 34.7 | Allium grande | N | R | 60.4 |
| Xanthium strumarium | G | S | 23.2 | Allium schoenoporasum | N | O | 65.8 |
| Zea mays | G | O | 20.1 | Allium schoenoprasum | N | R | 31.0 |
| Zea mays | G | S | 45.9 | Allium tuberosum | N | S | 22.8 |
| Zea mays | G | O | 97.5 | Allium tuberosum | N | O | 99.7 |
| Abelmochus esculentus | N | S | 24.8 | Althaea officinalis | N | S | 22.8 |
| Abies lasiocarpa | N | R | 44.7 | Althaea officinalis | N | O | 22.1 |
| Achillea millefolium | N | O | 24.1 | Amaranthus candathus | N | R | 43.9 |
| Achillea millefolium | N | S | 59.2 | Amaranthus gangeticus | N | O | 30.3 |
| Aconitum napellus | N | S | 40.6 | Amaranthus gangeticus | N | S | 66.0 |
| Aconitum napellus | N | O | 41.6 | Ambrosia artemisiifolia | N | R | 58.7 |
| Acorus calamus | N | O | 47.1 | Amelanchier alnitolia | N | R | 70.5 |
| Actinidia arguta | N | S | 21.8 | Amelanchier sanguinea | N | R | 37.3 |
| Adiantum pedatum | N | S | 26.8 | Ananas comosus | N | R | 23.8 |
| Adiantum pedatum | N | O | 45.8 | Ananas comosus | N | O | 95.0 |
| Adiantum pedatum | N | R | 86.0 | Ananas comosus | N | O | 99.6 |
| Agaricus bisporus | N | S | 26.3 | Angelica archangelica | N | S | 30.5 |
| Agaricus bisporus | N | O | 29.8 | Angelica archangelica | N | R | 38.9 |
| Agaricus bisporus | N | R | 36.9 | Anthemis nobilis | N | O | 41.4 |
| Anthemis nobilis | N | R | 72.8 | Averrhoa carambola | N | R | 23.4 |
| Anthemis tinctorium | N | S | 27.3 | Cyperus esculentus | N | S | 46.2 |
| Anthriscus cerefolium | N | R | 35.8 | Beta vulgaris | N | R | 28.2 |
| Apium graveolens | N | S | 31.7 | Beta vulgaris | N | S | 30.4 |
| Apium graveolens | N | R | 32.4 | Beta vulgaris | N | O | 56.8 |
| Apium graveolens | N | R | 56.6 | Beta vulgaris subsp.. | N | R | 23.6 |
| | | | | maritima | | | |
| Aralia cordata | N | R | 29.2 | Betula glandulosa | N | O | 22.2 |
| Aralia cordata | N | S | 45.0 | Betula glandulosa | N | O | 22.2 |
| Arctium minus | N | R | 25.8 | Betula glandulosa | N | S | 25.7 |
| Arctostaphylos uva-ursi | N | O | 31.0 | Betula glandulosa | N | R | 32.9 |
| Arctostaphylos uva-ursi | N | S | 35.2 | Boletus edulis | N | S | 36.2 |
| Arctostaphylos uva-ursi | N | R | 58.6 | Boletus edulis | N | O | 90.2 |
| Armoracia rusticana | N | R | 24.9 | Borago officinalis | N | S | 27.9 |
| Armoracia rusticana | N | S | 52.9 | Borago officinalis | N | O | 76.1 |
| Aronia melanocarpa | N | R | 40.0 | Brassica cepticepa | N | O | 65.4 |
| Aronia melanocarpa | N | O | 91.9 | Brassica cepticepa | N | S | 71.5 |
| Aronia prunifolia | N | R | 100.0 | Brassica Chinensis | N | R | 27.1 |
| Arrhenatherum elatius | N | R | 22.8 | Brassica juncea | N | O | 51.0 |
| Artemisia draculus | N | S | 74.9 | Brassica juncea | N | R | 66.0 |
| Artemisia dracunculus | N | S | 47.8 | Brassica juncea | N | S | 74.1 |
| Asclepias incarnata | N | R | 20.5 | Brassica napus | N | S | 22.0 |
| Asctinidia chinensis | N | O | 43.4 | Brassica napus | N | R | 34.0 |
| Asctinidia chinensis | N | O | 66.4 | Brassica napus | N | O | 100.0 |
| Asparagus officinalis | N | O | 91.3 | Brassica nigra | N | S | 26.7 |
| Asparagus officiralis | N | R | 23.3 | Brassica nigra | N | O | 27.4 |
| Asparagus officiralis | N | S | 44.7 | Brassica nigra | N | R | 82.5 |
| Aster Linné. | N | S | 47.5 | Brassica oleracea | N | O | 21.2 |
| Aster sp. | N | R | 62.0 | Brassica oleracea | N | S | 22.1 |
| Atriplex hortensis | N | R | 54.6 | Brassica oleracea | N | R | 26.2 |
| Atropa belladonna | N | R | 20.1 | Brassica oleracea | N | R | 27.2 |
| Atropa belladonna | N | S | 51.0 | Brassica oleracea | N | O | 31.3 |
| Avena sativa | N | R | 24.8 | Brassica oleracea | N | R | 46.5 |
| Avena sativa | N | R | 26.4 | Brassica oleracea | N | S | 71.2 |
| Brassica oleracea | N | O | 93.5 | Chrysanthenum | N | R | 38.2 |
| | | | | coronarium | | | |
| Brassica rapa | N | R | 25.6 | Chrysanthenum | N | S | 63.9 |
| | | | | coronarium | | | |
| Brassica rapa | N | R | 33.9 | Cicer arietinum | N | S | 20.0 |
| Brassica rapa | N | R | 56.0 | Cichorium endivia | N | S | 25.6 |
| Brassica rapa | N | S | 69.7 | Cichorium endivia crispa | N | O | 38.4 |
| Brassica rapa | N | O | 100.0 | Cichorium intybus | N | S | 30.2 |
| Bromus inermis | N | R | 57.3 | Cimicifuga racemosa | N | S | 33.7 |
| Campanula rapunculus | N | O | 77.5 | Citrullus colocynthus | N | S | 20.4 |
| Canne edulis | N | O | 75.6 | Citrullus lanatus | N | O | 68.3 |
| Cantharellus ciparium | N | O | 52.5 | Citrullus lanatus | N | S | 31.9 |
| Capsella bursa-pastoris | N | O | 35.9 | Citrus limettoides | N | R | 20.4 |
| Capsicum annuum | N | S | 43.9 | Citrus limettoides | N | O | 37.5 |
| Capsicum annuum | N | S | 50.1 | Citrus limon | N | O | 47.7 |
| Capsicum frutescens | N | S | 28.9 | Citrus limon | N | O | 72.4 |
| Carica papaya | N | R | 31.1 | Citrus paradisi | N | R | 23.8 |
| Carthamus tinctorius | N | R | 37.3 | Citrus paradisi | N | O | 33.4 |
| Carum carvi | N | S | 30.1 | Citrus reticulata | N | O | 20.4 |
| Castanea spp. | N | R | 21.7 | Citrus reticulata | N | O | 20.9 |
| Chaerophyllum | N | S | 46.0 | Citrus reticulata | N | R | 26.0 |
| bulbosum | | | | | | | |
| Chamaemelum nobile | N | R | 36.8 | Citrus reticulata | N | S | 40.4 |
| Chamaemelum nobile | N | R | 48.4 | Citrus reticulata | N | O | 50.0 |
| Chelidonium majus | N | O | 46.6 | Citrus reticulata | N | O | 79.2 |
| Chenapodium bonus- | N | R | 22.4 | Citrus sinensis | N | R | 25.3 |
| henricus | | | | | | | |
| Chenopodium bonus- | N | S | 57.6 | Citrus sinensis | N | O | 59.8 |
| henricus | | | | | | | |
| Chenopodium quinoa | N | O | 35.5 | Coix lacryma-jobi | N | R | 20.0 |
| Chenopodium quinoa | N | R | 54.4 | Corchorus olitorius | N | S | 38.9 |
| Chrysanthemum | N | R | 26.5 | Cornus canadensis | N | S | 35.6 |
| leucanthemum | | | | | | | |
| Chrysanthemun | N | R | 48.4 | Cosmos sulphureus | N | S | 51.4 |
| coronarium | | | | | | | |
| var. spatiosum | | | | | | | |
| Crataegus sp | N | O | 28.0 | Dioscorea batatas | N | O | 83.1 |
| | | | | Dioscorea batatas | N | O | 47.6 |
| Crataegus sp | N | R | 60.9 | Diospiros kaki | N | R | 34.9 |
| Crataegus submollis | N | O | 25.5 | Dirca palustris | N | S | 27.6 |
| Crithmum maritima | N | S | 50.6 | Dirca palustris | N | O | 90.4 |
| Cryptotaenia canadensis | N | O | 21.2 | Dolichos lablab | N | R | 66.4 |
| Cryptotaenia canadensis | N | R | 26.0 | Dolichos lablab | N | O | 85.3 |
| Cryptotaenia canadensis | N | O | 40.0 | Dryopteris filix-mas | N | S | 21.9 |
| Cucumis anguria | N | S | 38.7 | Dryopteris filix-mas | N | R | 77.9 |
| Cucumis anguria | N | O | 46.6 | Echinacea purpurea | N | S | 48.6 |
| Cucumis melo | N | S | 30.3 | Eleusine coracana | N | O | 45.2 |
| Cucumis melo | N | O | 46.2 | Elymus junceus | N | R | 41.0 |
| Cucumis metuliferus | N | R | 32.0 | Erigeron canadensis | N | S | 31.4 |
| Cucumis sativus | N | O | 40.3 | Eriobotrya japonica | N | R | 28.3 |
| Cucurbita maxima | N | S | 23.6 | Eruca vesicaria | N | R | 44.9 |
| Cucurbita maxima | N | S | 33.1 | Fagopyrum esculentum | N | R | 76.7 |
| Cucurbita maxima | N | O | 55.2 | Fagopyrum tartaricum | N | R | 42.6 |
| Cucurbita moschata | N | S | 20.1 | Festuca rubra | N | R | 29.6 |
| Cucurbita moschata | N | S | 26.7 | Festuca rubra | N | S | 42.9 |
| Cucurbita moschata | N | O | 41.7 | Foeniculum vulgare | N | O | 22.1 |
| Cucurbita pepo | N | S | 41.9 | Foeniculum vulgare | N | S | 21.6 |
| Cucurbita pepo | N | O | 82.9 | Foeniculum vulgare | N | O | 84.8 |
| Curcuma zedoaria | N | S | 100.0 | Forsythia x intermedia | N | O | 70.8 |
| Cydonia oblonga | N | R | 42.9 | Forsythia x intermedia | N | O | 60.2 |
| Cynara scolymus | N | R | 51.6 | Fortunella spp | N | S | 35.7 |
| Cynara scolymus | N | S | 60.9 | Fortunella spp | N | R | 50.7 |
| Dactilis glomerata | N | R | 25.7 | Fortunella spp | N | O | 74.5 |
| Datura stramonium | N | R | 21.9 | Fragaria | N | R | 24.8 |
| Daucus carota | N | R | 25.9 | Fragaria | N | O | 52.4 |
| Dioscorea batatas | N | O | 47.6 | Fragaria | N | O | 100.0 |
| Fragaria x ananassa | N | S | 29.3 | Hibiscus cannabinus | N | S | 48.9 |
| Galium odoratum | N | R | 26.0 | Hordeum vulgare | N | S | 29.2 |
| Gaultheria hispidula | N | R | 40.3 | Humulus lupulus | N | R | 22.4 |
| Ginkgo biloba | N | O | 27.0 | Humulus lupulus | N | R | 39.1 |
| Ginkgo biloba | N | R | 68.9 | Humulus lupulus | N | O | 63.1 |
| Glechoma hederacea | N | R | 20.4 | Humulus lupulus | N | S | 100.0 |
| Glechoma hederacea | N | S | 30.4 | Hydrastis canadensis | N | S | 20.2 |
| Glycine max | N | O | 26.6 | Hydrastis canadensis | N | R | 31.0 |
| Glycine max | N | R | 47.4 | Hyoscyamus niger | N | O | 56.8 |
| Glycine max | N | S | 82.0 | Hypericum henryi | N | O | 48.8 |
| Glycyrrhiza glabra | N | S | 35.4 | Hypericum perforatum | N | S | 48.1 |
| Glycyrrhiza glabra | N | O | 40.5 | Hypericum perforatum | N | O | 63.7 |
| Glycyrrhiza glabra | N | R | 100.0 | Hypomyces lactiflorum | N | S | 44.8 |
| Gossypium herbaceum | N | S | 36.1 | Hypomyces lactiflorum | N | O | 60.9 |
| Guizotia abyssinica | N | R | 28.9 | Hyssops officinalis | N | R | 22.9 |
| Guizotia abyssinica | N | S | 40.4 | Inula helenium | N | S | 24.6 |
| Hamamelis virginiana | N | O | 52.4 | Juniperus communis | N | S | 33.0 |
| Hamamelis virginiana | N | S | 67.5 | Juniperus communis | N | O | 38.2 |
| Hamamelis virginiana | N | R | 84.1 | Lactuca sativa | N | S | 44.5 |
| Hedeoma pulegiodes | N | S | 57.4 | Lactuca sativa | N | R | 50.7 |
| Helenium hoopesii | N | O | 33.7 | Laportea canadensis | N | S | 30.2 |
| Helenium hoopesii | N | S | 49.0 | Lathyrus sativus | N | O | 20.4 |
| Helianthus annuus | N | S | 53.4 | Lathyrus sativus | N | R | 52.5 |
| Helianthus strumosus | N | R | 20.3 | Lathyrus sylvestris | N | R | 27.7 |
| Helianthus strumosus | N | O | 71.7 | Lathyrus sylvestris | N | O | 36.8 |
| Helianthus tuberosa | N | R | 22.8 | Laurus nobilis | N | S | 52.0 |
| Helianthus tuberosus L. | N | O | 22.6 | Lavendula angustifolia | N | R | 26.4 |
| Helianthus tuberosus L. | N | S | 55.0 | Lavendula angustifolia | N | S | 53.2 |
| Helichrysum | N | S | 67.0 | Lavendula latifolia | N | S | 51.3 |
| angustifolium | | | | | | | |
| Heliotropium | N | S | 58.9 | Ledum groenlandicum | N | S | 44.4 |
| arborescens | | | | | | | |
| Helleborus niger | N | S | 31.9 | Lentinus edodes | N | R | 42.1 |
| Lentinus edodes | N | O | 100.0 | Manihot esculenta syn. | N | O | 86.5 |
| | | | | M. utilissima | | | |
| Lepidium sativum | N | S | 44.2 | Manihot esculenta syn. | N | S | 50.4 |
| | | | | M. utilissima | | | |
| Levisticum officinale | N | S | 20.8 | Melilotus alba | N | R | 30.4 |
| Levisticum officinale | N | O | 39.4 | Melilotus officinalis | N | R | 68.1 |
| Linum usitatissimum | N | R | 42.3 | Melissa officinalis | N | S | 33.7 |
| Litchi chinensis | N | R | 25.7 | Melissa officinalis | N | O | 34.7 |
| Lolium multiflorum | N | S | 20.6 | Mentha arvensis | N | R | 53.7 |
| Lolium perenne | N | R | 28.7 | Mentha suaveolens | N | S | 26.8 |
| Lonicera ramosissima | N | S | 26.3 | Menyanthes trifoliata | N | S | 32.8 |
| Lonicera ramosissima | N | O | 40.4 | Miscanthus sinensis | N | R | 22.7 |
| | | | | Andress | | | |
| Lonicera ramosissima | N | R | 53.2 | Momordica charantia | N | S | 55.5 |
| Lonicera syringantha | N | R | 95.8 | Monarda didyma | N | S | 26.8 |
| Lotus corniculatus | N | R | 100.0 | Monarda fistulosa | N | S | 21.5 |
| Lotus tetragonolubus | N | S | 65.4 | Montia perfoliata | N | R | 26.6 |
| Lunaria annua | N | O | 55.7 | Musa paradisiaca | N | R | 29.0 |
| Lunaria annua | N | S | 67.3 | Nasturtium officinale | N | S | 35.4 |
| Lycopersicon esculentum | N | R | 37.6 | Nepeta cataria | N | R | 26.5 |
| Malus sp. | N | R | 31.8 | Nepeta cataria | N | O | 27.5 |
| Malus sp. | N | O | 44.4 | Nepeta cataria | N | S | 41.9 |
| Malus hupehensis | N | R | 26.3 | Nephelium longana | N | R | 43.4 |
| (Pamp.) Rehd. | | | | | | | |
| Malus hupehensis | N | S | 67.0 | Nicotiana rustica | N | O | 26.0 |
| (Pamp.) Rehd. | | | | | | | |
| Malus sp. | N | R | 65.3 | Nicotiana rustica | N | S | 32.7 |
| Malva moschata | N | S | 41.1 | Nicotiana tabacum | N | S | 25.1 |
| Malva sylvestris | N | S | 36.4 | Nicotiana tabacum | N | O | 77.7 |
| Malva sylvestris | N | O | 47.4 | Nigella sativa | N | R | 59.3 |
| Malva verticillata | N | R | 42.7 | Nigella sativa | N | R | 100.0 |
| Mangifera indica | N | O | 30.5 | Ocimum basilicum | N | R | 20.2 |
| Manihot esculenta syn. | N | R | 38.3 | Ocimum basilicum | N | O | 20.2 |
| M. utilissima | | | | | | | |
| Ocimum basilicum | N | S | 32.8 | Phoenix dactylifera | N | O | 29.6 |
| Oenothera biennis Linné | N | R | 100.0 | Physalis alkekengi | N | R | 32.9 |
| Onobrychis viciafolia | N | R | 45.0 | Physalis ixocarpa | N | R | 26.6 |
| Optunia sp. | N | R | 33.4 | Physalis ixocarpa | N | O | 28.3 |
| Origanum marjonara | N | O | 20.5 | Physalis pruinosa | N | S | 27.3 |
| Origanum vulgare | N | O | 20.8 | Physalis pruinosa | N | R | 47.8 |
| Origanum vulgare | N | R | 21.6 | Physalis pruinosa | N | O | 93.1 |
| Oryza sativa | N | R | 42.4 | Physalis sp. | N | R | 39.1 |
| Oxyria digyna | N | O | 57.0 | Physalis sp. | N | O | 60.8 |
| Oxyria digyna | N | O | 77.9 | Phytolacca americana | N | S | 41.8 |
| Panax quinquefolius L. | N | O | 23.5 | Phytolacca americana | N | O | 100.0 |
| Panicum miliaceum | N | R | 36.5 | Phytolacca decandra syn. | N | O | 85.9 |
| | | | | P. americana | | | |
| Passiflora spp | N | S | 35.8 | Pimpinella anisum | N | S | 20.2 |
| Passiflora spp | N | O | 38.3 | Pimpinella anisum | N | O | 68.4 |
| Passiflora spp | N | R | 46.2 | Pisum sativum | N | R | 20.1 |
| Passiflora spp | N | O | 100.0 | Pisum sativum | N | S | 25.8 |
| Pastinaca sativa | N | O | 21.7 | Pisum sativum | N | O | 27.0 |
| Pastinaca sativa | N | R | 38.6 | Pisum sativum | N | O | 51.8 |
| Pastinaca sativa | N | S | 39.2 | Plantago coronopus | N | R | 21.9 |
| Persea americana | N | O | 32.5 | Plantago coronopus | N | O | 48.6 |
| Persea americana | N | O | 38.6 | Plantago coronopus | N | S | 66.8 |
| Petasites japonicus | N | S | 26.2 | Plantago major | N | S | 35.1 |
| Phalaris canariensis | N | O | 80.0 | Pleurotus spp | N | R | 25.3 |
| Phaseolus coccineus | N | S | 44.4 | Pleurotus spp | N | S | 59.3 |
| Phaseolus coccineus | N | R | 79.1 | Pleurotus spp | N | O | 85.2 |
| Phaseolus mungo | N | S | 27.0 | Poa compressa | N | R | 26.2 |
| Phaseolus mungo | N | O | 37.9 | Poa pratensis | N | O | 21.5 |
| Phaseolus vulgaris | N | R | 20.1 | Poa pratensis | N | R | 30.0 |
| Phaseolus vulgaris | N | S | 51.9 | Podophyllum peltatum | N | O | 33.9 |
| Phaseolus vulgaris | N | O | 61.7 | Podophyllum peltatum | N | S | 50.2 |
| Phlox paniculata | N | S | 22.9 | Polygonum aviculare | N | R | 31.0 |
| | | | | Linné | | | |
| Phlox paniculata | N | O | 44.5 | Polygonum | N | S | 56.6 |
| | | | | pennsylvanicum | | | |
| Polygonum persicaria | N | S | 20.1 | Rheum officinale | N | S | 100.0 |
| Populus incrassata | N | R | 54.9 | Rheum palmatum | N | R | 20.2 |
| Populus tremula | N | R | 31.0 | Rheum rhabarbarum | N | S | 33.8 |
| Populus x petrowskyana | N | R | 100.0 | Ricinus communis | N | S | 20.9 |
| Potentilla anserina | N | S | 22.1 | Ribes nidigrolaria | N | R | 44.5 |
| Potentilla anserina | N | O | 41.1 | Ribes nidigrolaria | N | O | 53.1 |
| Prunus cerasus | N | O | 30.1 | Ribes nigrum | N | S | 40.7 |
| Prunus persica | N | R | 26.6 | Ribes nigrum L. | N | R | 50.0 |
| Prunus persica | N | O | 38.5 | Ribes nigrum L. | N | O | 60.1 |
| Prunus spp | N | S | 24.0 | Ribes sativum | N | R | 47.9 |
| Prunus spp | N | O | 49.1 | Ribes sativum | N | R | 48.2 |
| Psidium guajaba | N | O | 22.5 | Ribes silvestre | N | O | 26.3 |
| Psidium guajaba | N | R | 44.3 | Ribes silvestre | N | R | 100.0 |
| Psidium guajaba | N | O | 95.4 | Ribes uva-crispa | N | O | 57.5 |
| Psidium spp | N | S | 36.6 | Rosa rugosa | N | S | 27.8 |
| Psidium spp | N | R | 47.6 | Rosa rugosa thunb. | N | R | 37.5 |
| Psidium spp | N | O | 87.6 | Rosa rugosa thunb. | N | O | 45.7 |
| Pteridium aquilinum | N | R | 22.0 | Rosmarinum officinalis | N | R | 44.2 |
| Punica granatum | N | O | 52.1 | Rosmarinum officinalis | N | R | 65.9 |
| Pyrus communis | N | O | 39.5 | Rubus canadensis | N | S | 45.5 |
| Pyrus pyrifolia | N | R | 33.7 | Rubus idaeus | N | R | 31.4 |
| Raphanus raphanistrum | N | O | 24.5 | Rubus idaeus | N | O | 57.2 |
| Raphanus raphanistrum | N | S | 44.8 | Rubus idaeus | N | S | 28.5 |
| Raphanus raphanistrum | N | S | 46.1 | Rubus idaeus | N | O | 38.0 |
| Raphanus sativus | N | O | 25.4 | Rubus occidentalis | N | O | 21.4 |
| Raphanus sativus | N | R | 32.1 | Rubus occidentalis | N | S | 36.5 |
| Raphanus sativus | N | R | 38.1 | Rubus occidentalis | N | R | 60.2 |
| Raphanus sativus | N | S | 63.6 | Rumex scutatus | N | O | 84.5 |
| Raphanus sativus | N | O | 93.4 | Rumex crispus Linné | N | O | 52.5 |
| Reseda luteola | N | S | 22.5 | Rumex crispus Linné | N | R | 100.0 |
| Rhamnus frangula | N | S | 34.2 | Rumex patientia | N | O | 23.1 |
| Rhamnus frangula | N | R | 39.5 | Rumex patientia | N | S | 65.8 |
| Ruta graveolens | N | S | 37.2 | Solanum melogena | N | S | 67.1 |
| Sabal serrulata syn. | N | O | 34.4 | Solanum Tuberosum | N | O | 68.6 |
| Serenoa repens | | | | | | | |
| Sabal serrulata syn. | N | S | 44.6 | Solidago canadensis | N | S | 48.4 |
| Serenoa repens | | | | | | | |
| Salix purpurea | N | R | 67.8 | Solidago sp | N | R | 31.4 |
| Salvia elegens | N | O | 51.1 | Solidago virgaurea | N | S | 56.2 |
| Sambucus canadensis | N | S | 44.8 | Sorghum caffrorum | N | O | 23.3 |
| Sambucus canadensis | N | O | 72.4 | Sorghum dochna bicolor | N | R | 20.8 |
| | | | | gr technicum | | | |
| Sambucus canadensis L. | N | R | 67.8 | Sorghum dochna var. | N | S | 21.4 |
| | | | | snowdrew | | | |
| Sambucus ebulus | N | O | 44.3 | Sorghum dochna var. | N | O | 27.7 |
| | | | | snowdrew | | | |
| Sanguisorba officinalis | N | R | 100.0 | Spinacia oleracea | N | O | 25.0 |
| Santolina | N | R | 37.9 | Spinacia oleracea | N | R | 32.1 |
| Satureja montana | N | S | 20.0 | Spinacia oleracea | N | S | 47.6 |
| Satureja montana | N | O | 21.3 | Spinacia oleracea | N | O | 63.1 |
| Satureja repandra | N | S | 36.3 | Stachys affinis | N | R | 31.7 |
| Scorzorera hipanica | N | R | 27.1 | Stachys affinis | N | O | 100.0 |
| Scorzorera hipanica | N | S | 31.7 | Stachys byzantina | N | R | 30.9 |
| Scuttellaria lateriflora | N | S | 44.3 | Stipa capillata L. | N | R | 20.1 |
| Secale cereale | N | S | 24.2 | Symphytum officinale | N | S | 24.1 |
| Secale cereale | N | R | 31.1 | Tanacetum | N | O | 24.2 |
| | | | | Cinerarifolium | | | |
| Sechium edule | N | S | 37.8 | Tanacetum | N | R | 84.4 |
| | | | | Cinerarifolium | | | |
| Sesamum indicum | N | S | 59.2 | Tanacetum vulgare | N | R | 25.7 |
| Setaria italica | N | R | 33.0 | Tanacetum vulgare | N | S | 75.6 |
| Silybum marianum | N | O | 92.4 | Taraxacum officinale | N | S | 21.1 |
| | | | | (Red ribe) | | | |
| Sium sisarum | N | O | 32.7 | Phaseolus acutifolius var. | N | R | 56.7 |
| | | | | latifolius | | | |
| Sium sisarum | N | S | 33.1 | Teucrium chamaedrys L. | N | R | 27.3 |
| Sium sisarum | N | O | 81.3 | Thlaspi arvense | N | S | 61.4 |
| Solanum melogena | N | O | 21.9 | Thymus fragantissumus | N | R | 100.0 |
| solanum melogena | N | O | 26.1 | Thymus herba-barona | N | R | 22.0 |
| Solanum melogena | N | R | 34.0 | Thymus | N | R | 36.8 |
| | | | | pseudolanuginosus | | | |
| Thymus | N | S | 37.1 | Vaccinium angustifolium | N | O | 25.2 |
| pseudolanuginosus | | | | | | | |
| Thymus serpyllum | N | S | 26.0 | Vaccinium angustifolium | N | R | 34.6 |
| Thymus serpyllum | N | R | 42.7 | Vaccinium angustifolium | N | O | 59.6 |
| Thymus x citriodorus | N | O | 22.7 | Vaccinium angustifolium | N | R | 65.7 |
| Tiarella cordifolia | N | R | 100.0 | Vaccinium macrocarpon | N | O | 30.2 |
| Tragopogon porrifolius | N | O | 26.8 | Vaccinium macrocarpon | N | S | 39.0 |
| Tragopogon porrifolius | N | O | 28.4 | Vaccinium macrocarpon | N | S | 56.9 |
| Tragopogon porrifolius | N | O | 42.1 | Vaccinum macrocarpon | N | O | 39.2 |
| Tragopogon sp. | N | O | 20.3 | Vaccinum macrocarpon | N | R | 42.3 |
| Tragopogon sp. | N | S | 32.0 | Veratrum viride | N | O | 20.5 |
| Tragopogon sp. | N | R | 66.3 | Veratrum viride | N | S | 33.1 |
| Trichosanthes kirilowii | N | O | 66.5 | Verbascum thapsus | N | S | 43.1 |
| Trifolium incarnatum | N | R | 47.9 | Verbascum thapsus | N | O | 70.2 |
| Trifolium repens | N | R | 81.7 | Veronica officinalis | N | O | 20.5 |
| Trigonella foenum | N | S | 39.6 | Viburnum trilobum | N | S | 40.6 |
| graecum | | | | Marsh. | | | |
| xTriticosecale sp. | N | O | 64.1 | Vicia faba | N | R | 61.5 |
| Triticum aestivum | N | R | 24.5 | Vicia sativa | N | R | 30.1 |
| Triticum aestivum | N | S | 29.4 | Vigna angularia | N | R | 32.6 |
| Triticum furgidumm | N | S | 35.8 | Vigna angularia | N | S | 64.2 |
| Triticum spelta | N | S | 34.7 | Vigna unguiculata | N | R | 32.4 |
| Tropaeolum majus | N | O | 90.3 | Vigna unguiculata | N | O | 47.4 |
| Tropaeolum malus | N | R | 20.1 | Vigna unguiculata | N | S | 51.0 |
| Tsuga canadensis | N | O | 21.5 | Vinca minor | N | S | 21.3 |
| Tsuga canadensis | N | R | 64.4 | Vitis sp. | N | O | 28.3 |
| Tsuga diversifolia | N | O | 45.9 | Vitis sp. | N | O | 29.4 |
| Tsuga diversifolia | N | R | 100.0 | Vitis sp. | N | S | 45.4 |
| Tsuga F. macrophylla | N | R | 28.1 | Vitis sp. | N | O | 50.7 |
| Typha latifolia L. | N | S | 30.6 | Vitis sp. | N | R | 61.6 |
| Urtica dioica | N | O | 31.4 | Vitis sp. | N | R | 100.0 |
| Urtica dioica | N | R | 36.9 | Weigela coracensis | N | R | 35.5 |
| Urtica dioica | N | S | 41.7 | Withania somnifera | N | S | 35.5 |
| Xanthium sibiricum | N | S | 38.6 | Zingiber officinale | N | S | 20.1 |
| Xanthium strumarium | N | S | 33.5 | Zingiber officinale | N | R | 58.9 |
| Zea mays | N | S | 37.1 | Zingiber officinale | N | O | 75.9 |
| Zea mays | N | O | 65.5 | | | | |

TABLE 7
Plant Extracts Capable of Inhibiting Cathepsin B

| | | | Inhibition | | | | Inhibition |
|---|---|---|---|---|---|---|---|
| Latin name | Str | Extr | (%) | Latin name | Str | Extr | (%) |
| Achillea millefolium | A | O | 61.9 | Athyrium asperum | A | O | 27.3 |
| Achillea tomentosa | A | O | 60.8 | Atropa belladonna | A | O | 37.7 |
| Aconitum | A | O | 38.6 | Begonia convolvulacea | A | O | 26.0 |
| Aconitum napellus | A | O | 61.1 | Begonia eminii | A | O | 34.2 |
| Alchemilla mollis | A | R | 26.7 | Begonia glabra | A | O | 38.9 |
| Allium | A | R | 43.0 | Begonia Hannii | A | O | 52.9 |
| Allium cepa gr. Cepa | A | O | 49.9 | Begonia polygonoides | A | O | 67.3 |
| Allium cepa gr. Cepa | A | O | 70.1 | Berberis vulgaris | A | O | 54.6 |
| Allium cepa gr. Cepa | A | R | 45.8 | Beta vulgaris | A | R | 39.9 |
| Allium sativum | A | O | 25.6 | Beta vulgaris | A | R | 30.4 |
| Allium Tuberosum | A | O | 91.5 | Beta vulgaris | A | O | 61.9 |
| Allium Tuberosum | A | O | 75.0 | Beta vulgaris | A | O | 43.0 |
| Allium victorialis | A | O | 31.1 | Beta vulgaris | A | R | 91.0 |
| Amaranthus gangeticus | A | O | 26.1 | Beta vulgaris | A | O | 46.7 |
| Amaranthus gangeticus | A | O | 29.0 | Beta vulgaris | A | R | 65.3 |
| Amelanchier canadensis | A | R | 28.7 | Beta vulgaris | A | R | 33.4 |
| Anthemis tinctoria | A | O | 26.8 | Beta vulgaris | A | O | 54.3 |
| Anthemis tinctoria | A | R | 32.4 | Beta vulgaris | A | O | 38.2 |
| Anthoxanthum odoratum | A | O | 24.9 | Beta vulgaris | A | R | 55.9 |
| Apium graveolens | A | O | 31.1 | Beta vulgaris | A | R | 28.5 |
| Apium graveolens | A | O | 20.6 | Beta vulgaris | A | O | 40.1 |
| Aralia cordata | A | R | 52.3 | Beta vulgaris spp. | A | O | 33.4 |
| | | | | Maritima | | | |
| Arctium lappa | A | O | 33.7 | Brassica juncea | A | O | 21.3 |
| Arctium lappa | A | R | 33.0 | Brassica Oleracea | A | O | 27.5 |
| Aronia melanocarpa | A | R | 41.2 | Brassica Oleracea | A | O | 48.2 |
| (Michx.) Ell. | | | | | | | |
| Aronia melanocarpa | A | O | 21.6 | Brassica rapa | A | O | 20.8 |
| (Michx.) Ell. | | | | | | | |
| Asarum europaeum | A | O | 24.9 | Calendula officinalis | A | O | 35.6 |
| Athaea officinalis | A | O | 57.7 | Camellia sinensis | A | R | 24.4 |
| Cana edulis | A | R | 100.0 | Geum rivale | A | O | 26.4 |
| Capsicum annuum | A | O | 25.0 | Glycyrrhiza glabra | A | R | 86.8 |
| Capsicum frutescens | A | O | 29.6 | Heliotropium | A | O | 29.5 |
| | | | | arborescens | | | |
| Chrysanthemum | A | O | 89.3 | Humulus Lupulus | A | O | 65.4 |
| balsamita | | | | | | | |
| Chrysanthemun | A | O | 55.0 | Humulus Lupulus | A | R | 100.0 |
| balsamina | | | | | | | |
| Chrysanthemun | A | O | 30.1 | Hylotelephium | A | R | 23.7 |
| coronarium (Chp Suey) | | | | | | | |
| Chrysanthemun | A | O | 36.4 | Hypericum henryi | A | R | 44.4 |
| coronarium (Chp Suey) | | | | | | | |
| Cichorium intybus | A | R | 100.0 | Iberis sempervirens | A | O | 84.6 |
| Citrullus lanatus | A | O | 24.4 | Jeffersonia diphylla | A | O | 35.4 |
| Convallaria maialis | A | O | 57.0 | Ligularia dentata | A | O | 30.3 |
| Coriandrum sativum | A | R | 20.8 | Lonicera ramosissima | A | R | 48.7 |
| Cryptotaenia canadensis | A | O | 20.4 | Miscanthus | A | O | 50.9 |
| | | | | sacchariflorus | | | |
| Cucumis Anguria | A | O | 26.8 | Nicotiana tabacum | A | O | 40.0 |
| Cucumis sativus | A | R | 45.6 | Nicotiana tabacum | A | O | 56.8 |
| Curburbita pepo | A | O | 30.8 | Nicotiana tabacum | A | O | 55.2 |
| Daucus carota | A | R | 68.8 | Nigella sativa | A | O | 40.3 |
| Daucus carota | A | O | 20.3 | Origanum majorana | A | O | 49.7 |
| Daucus carota | A | R | 72.5 | Origanum vulgare | A | O | 67.0 |
| Daucus carota | A | O | 22.6 | Origanum vulgare | A | O | 39.9 |
| Daucus carota | A | O | 25.6 | Panax quinquefolius L. | A | O | 24.0 |
| Daucus carota | A | R | 65.9 | Pastinaca sativa | A | R | 33.5 |
| Daucus carota | A | R | 77.3 | Petroselinum crispum | A | O | 70.2 |
| Daucus carota | A | R | 41.6 | Peucedanum cervaria | A | O | 21.5 |
| Dirca palustris | A | R | 100.0 | Phaseolus Vulgaris | A | O | 67.9 |
| Eruca vesicaria | A | O | 41.4 | Philadelphus coronarius | A | O | 24.0 |
| Filipendula rubra | A | R | 65.0 | Physostegia virginiana | A | O | 56.9 |
| Forsythia intermedia | A | R | 100.0 | Phytolacca americana | A | O | 100.0 |
| Forsythia x intermedia | A | R | 100.0 | Plantago major | A | O | 31.2 |
| Plectranthus fruticosus | A | O | 32.1 | Thymus praecox subsp | A | O | 23.9 |
| | | | | arctitus | | | |
| Polygonum | A | R | 70.1 | Tiarella | A | R | 34.4 |
| pennsylvanicum | | | | | | | |
| Pulmonaria saccharata | A | O | 31.1 | Vaccinum augustifolium | A | R | 67.2 |
| Raphanus sativus | A | O | 21.5 | Vaccinum macrocarpon | A | R | 37.1 |
| Raphanus sativus | A | O | 50.5 | Vitia sp. | A | R | 93.7 |
| Raphanus sativus | A | O | 58.9 | Xanthium strumarium | A | O | 83.2 |
| Ribes nigrum L. | A | O | 53.1 | Yucca filamentosa | A | O | 34.5 |
| Rubus Allegheniensis | A | O | 56.7 | Zea mays | A | O | 29.7 |
| Rubus ideaus | A | R | 89.0 | Zea mays | A | O | 93.2 |
| Rumex crispus linné | A | R | 65.2 | Achillea tomentosa | G | O | 41.0 |
| Salvia elegens | A | O | 32.6 | Adiantum tenerum | G | R | 30.2 |
| Salvia nemorosa | A | O | 26.2 | Alcea rosea | G | O | 37.7 |
| Salvia officianalis | A | O | 26.3 | Alchemilla mollis | G | R | 32.8 |
| Salvia sclarea | A | R | 51.6 | Allium schoenoporasum | G | O | 49.3 |
| Salvia sclarea | A | O | 21.5 | Allium tuberosum | G | O | 79.1 |
| Saponaria officinalis | A | O | 68.5 | Allium tuberosum | G | O | 77.4 |
| Satureja montana | A | O | 47.6 | Allium victorialis | G | O | 45.5 |
| Scorzonera hispanica | A | O | 29.9 | Althaea officinalis | G | O | 67.2 |
| Sesamum indicum | A | O | 84.8 | amaranthus gangeticus | G | O | 23.5 |
| Solanum dulcamara | A | O | 51.3 | Anaphalis margaritacea | G | R | 34.7 |
| Solidago canadensis | A | O | 95.3 | Angelica dahurica | G | R | 27.9 |
| Solidago hybrida | A | O | 94.5 | Anthemis nobilis | G | O | 42.3 |
| Solidago hybrida | A | O | 99.5 | Apium graveolens | G | O | 25.7 |
| Solidago sp. | A | O | 60.9 | Apium graveolens | G | O | 27.4 |
| Stellaria graminea linné | A | O | 40.2 | Arctostaphylos uva-ursi | G | R | 94.5 |
| Tamarindus indica | A | O | 59.2 | Aronia melanocarpa | G | R | 74.5 |
| Taraxacum officinale | A | O | 88.6 | Aronia melanocarpa | G | O | 21.3 |
| Thalictrum | A | O | 65.2 | Aronia melanocarpa | G | R | 79.9 |
| aquilegiifolium | | | | (Michx.) Ell. | | | |
| Thalictrum | A | O | 44.5 | Aronia melanocarpa | G | R | 28.3 |
| Aquilegiifolium | | | | (Michx.) Ell. | | | |
| Thuja occidentalis | A | O | 50.6 | Asarum europaeum | G | O | 55.4 |
| Atropa belladonna | G | O | 58.9 | Filipendula rubra | G | R | 100.0 |
| Begonia eminii | G | O | 24.7 | Filipendula ulmaria | G | O | 20.5 |
| Begonia glabra | G | O | 42.9 | Filipendula vulgaris | G | O | 26.2 |
| Begonia manii | G | O | 32.1 | Forsythia intermedia | G | R | 100.0 |
| Begonia polygonoides | G | O | 38.2 | Forsythia x intermedia | G | R | 100.0 |
| Berberis vulgaris | G | O | 42.3 | Galium odoratum | G | O | 21.0 |
| Beta vulgaris | G | R | 75.3 | Gaultheria hispidula (L.) | G | R | 39.3 |
| | | | | Muhl | | | |
| Beta vulgaris | G | O | 28.7 | Gaultheria procumbens | G | R | 43.4 |
| Beta vulgaris | G | O | 21.7 | Geum rivale | G | O | 21.7 |
| Beta vulgaris | G | R | 40.0 | Glycine max | G | O | 64.2 |
| Beta vulgaris spp. | G | O | 31.4 | Glycyrrhiza glabra | G | R | 53.4 |
| Maritima | | | | | | | |
| Betula glandulosa | G | R | 38.5 | Hamamelis virginiana | G | R | 88.4 |
| Calendula officinalis | G | O | 36.2 | Heliotropium | G | O | 23.0 |
| | | | | arborescens | | | |
| Capsicum annus | G | O | 49.9 | Humulus lupulus | G | R | 100.0 |
| Chrysanthemum | G | O | 100.0 | Humulus lupulus | G | O | 90.2 |
| balsamita | | | | | | | |
| Chrysanthemun | G | O | 33.1 | Hydrastis canadensis | G | O | 30.9 |
| balsamina | | | | | | | |
| Cynara scolymus | G | O | 51.9 | Hylotelephium | G | R | 43.8 |
| Daucus carota | G | O | 81.3 | Hypericum henryi | G | R | 50.3 |
| Daucus carota | G | O | 27.2 | Iberis sempervirens | G | O | 87.7 |
| Dirca palustris | G | R | 100.0 | Lathyrus sativus | G | R | 25.9 |
| Echinacea purpurea | G | O | 22.9 | Ligularia dentata | G | O | 31.5 |
| Equisetum hyemale | G | O | 100.0 | Lunaria annua | G | O | 59.7 |
| Erigeron canadensis | G | O | 73.3 | Lythrum salicaire | G | R | 33.1 |
| Erigeron speciosus | G | O | 22.9 | Melissa officinalis | G | O | 27.6 |
| (Lindl.) D.C. | | | | | | | |
| Eruca vesicaria | G | O | 29.2 | Miscanthus | G | O | 30.7 |
| | | | | sacchariflorus | | | |
| Erysimum perofskianum | G | O | 89.8 | Nicotiana rustica | G | O | 54.8 |
| Fish. S. | | | | | | | |
| Foeniculum purpureum | G | R | 23.7 | Nicotiana tabacum | G | O | 36.2 |
| Filipendula rubra | G | R | 93.2 | Nigella sativa | G | O | 40.3 |
| Origan | G | O | 98.8 | Tamarindus indica | G | O | 65.4 |
| Origanum majorana | G | O | 48.9 | Taraxacum officinale | G | O | 82.7 |
| Panax quinquefolius L. | G | O | 21.1 | taraxacum officinale | G | O | 42.7 |
| Panicum miliaceum | G | R | 100.0 | Tetradenia riparia | G | O | 32.5 |
| Passiflora caerula | G | O | 66.2 | Thalictrum | G | O | 62.1 |
| | | | | aquilegiifolium | | | |
| Petroselinum crispum | G | O | 65.0 | Thuja occidentalis | G | O | 57.7 |
| Phaseolus vulgaris | G | R | 40.3 | Thymus vulgaris | G | O | 40.7 |
| | | | | “Argenteus” | | | |
| Physostegia virginiana | G | O | 74.0 | Tiarella | G | R | 39.0 |
| Phytolacca americana | G | O | 100.0 | Tropaeolum majus | G | O | 36.6 |
| Plantago major | G | O | 60.9 | Tussilago farfara | G | O | 26.8 |
| Plectranthus fruticosus | G | O | 29.2 | Vaccinium angustifolium | G | R | 26.4 |
| Polygonum aviculare | G | R | 45.6 | Vaccinium angustifolium | G | R | 89.1 |
| linné | | | | | | | |
| Pongamia pinnata | G | O | 41.7 | Vaccinum macrocarpon | G | R | 33.9 |
| Pulmonaria officinalis | G | O | 36.9 | Vitia sp. | G | R | 100.0 |
| Pulmonaria saccharata | G | O | 24.7 | Vitia sp. | G | R | 90.9 |
| Raphanus sativus | G | O | 38.9 | Vitis sp. | G | O | 37.1 |
| Raphanus sativus | G | O | 86.4 | Achillea millefolium | N | O | 44.1 |
| Rhus aromatica | G | O | 49.1 | Aconitum napellus | N | O | 27.4 |
| Ribes nigrum L. | G | O | 20.6 | Aesculus hippocastanum | N | R | 84.2 |
| Rubus ideaus | G | R | 56.9 | Aesculus hippocastanum | N | O | 47.3 |
| Rubus occidentalis | G | R | 61.3 | Alcea rosea “Nigra” | N | O | 24.3 |
| Saponaria officinalis | G | O | 48.3 | Alchemilla mollis | N | R | 24.9 |
| Sarriette vivace | G | O | 44.6 | Allium ascalonicum | N | O | 31.1 |
| Satureja repandra | G | O | 72.3 | Allium cepa gr. Cepa | N | O | 39.4 |
| Sesamum indicum | G | O | 46.8 | Allium cepa gr. Cepa | N | R | 23.2 |
| Sidalcea | G | O | 55.2 | Allium cepa gr. Cepa | N | O | 45.5 |
| Silene vulgaris | G | O | 35.5 | Allium fistulosum | N | O | 21.9 |
| Solanum dulcamara | G | O | 56.9 | Allium grande | N | O | 39.5 |
| Solidago canadensis | G | O | 99.8 | Allium tuberosum | N | O | 26.6 |
| Solidago canadensis | G | O | 100.0 | Allium tuberosum | N | O | 33.1 |
| Solidago sp. | G | O | 71.8 | Allium tuberosum | N | O | 72.3 |
| Sorghum caffrorum | G | O | 34.5 | Allium tuberosum | N | R | 22.6 |
| Allium victorialis | N | O | 42.3 | Begonia eminii | N | O | 40.4 |
| Alpinia oficinarum | N | O | 57.4 | Begonia glabra | N | O | 84.3 |
| Alpinia oficinarum | N | R | 88.9 | Begonia manii | N | O | 64.2 |
| Althacea officinalis | N | O | 51.5 | Berberus vulgaris | N | O | 35.4 |
| Althaea officianalis | N | O | 25.2 | Beta vulgaris | N | O | 34.1 |
| Amelanchier canadensis | N | O | 20.8 | Beta vulgaris | N | R | 86.7 |
| Amelanchier canadensis | N | R | 42.1 | Beta vulgaris | N | O | 23.8 |
| Amsonia | N | O | 30.2 | Beta vulgaris | N | R | 79.4 |
| tabernaemontana | | | | | | | |
| Ananas comosus | N | R | 36.2 | Beta vulgaris | N | O | 34.2 |
| Anaphalis margaritacea | N | R | 33.9 | Beta vulgaris | N | R | 20.8 |
| Angelica dahurica | N | R | 40.7 | Beta vulgaris | N | R | 37.0 |
| Angelica sinensis syn. A. polymorpha | N | O | 91.0 | Beta vulgaris spp. | N | R | 83.6 |
| | | | | Maritima | | | |
| Anthriscus cerefolium | N | R | 23.3 | Betula glandulosa | N | R | 62.5 |
| Anthriscus cerefolium | N | O | 21.7 | Borago officinalis | N | O | 23.5 |
| Aralia cordata | N | R | 44.1 | Brassica Napus | N | O | 27.6 |
| Aronia melanocarpa | N | R | 33.1 | Brassica oleracea | N | O | 21.8 |
| Aronia melanocarpa | N | R | 100.0 | Brassica oleracea | N | O | 22.3 |
| Aronia melanocarpa | N | R | 35.0 | Butomus umbellatus | N | O | 20.8 |
| (Michx.) Ell. | | | | | | | |
| Aronia prunifolia | N | R | 50.4 | Canna edulis | N | R | 100.0 |
| Artemisia draculus | N | O | 42.5 | Cinnamomum sp. | N | R | 99.5 |
| Asarum europaeum | N | O | 39.4 | Carica papaya | N | R | 100.0 |
| Asclepias incarnata L. | N | O | 48.7 | Chrysanthemum | N | O | 89.3 |
| | | | | balsamita | | | |
| Asclepias tuberosa | N | O | 21.5 | Chrysanthemum | N | R | 44.6 |
| | | | | parthenium | | | |
| Asctinidia chinensis | N | O | 24.9 | chrysanthemun | N | O | 28.7 |
| | | | | coronarium (Chp Suey) | | | |
| Atriplex hortensis | N | O | 22.4 | chrysanthemun | N | O | 59.2 |
| | | | | coronarium (Chp Suey) | | | |
| Atropa belladonna | N | O | 94.1 | Citrus paradisi | N | R | 100.0 |
| Crataegus oxyacantha | N | R | 72.7 | Citrus sinensis | N | R | 100.0 |
| Begonia convolvulacea | N | O | 32.1 | Cocos nucifera | N | R | 100.0 |
| Cocos nucifera | N | O | 71.9 | Humulus lupulus | N | O | 100.0 |
| Convallaria majalis | N | O | 67.1 | Humulus lupulus | N | R | 100.0 |
| Corchorus olitorius | N | R | 26.0 | Hydrastis canadensis | N | I | 42.7 |
| Crataegus sanguinea | N | O | 33.1 | Hypericum henryi | N | R | 51.8 |
| Cryptotaenia canadensis | N | R | 23.1 | Hypericum perforatum | N | O | 52.3 |
| Cucumis anguria | N | O | 26.4 | Hypomyces lactiflorum | N | O | 30.1 |
| Cucumis sativus | N | O | 25.7 | Iberis sempervirens | N | O | 90.8 |
| (Fanfare) | | | | | | | |
| Cydonia oblonga | N | R | 23.6 | Jeffersonia diphylla | N | O | 43.0 |
| Datura stramonium | N | O | 61.4 | Juglans nigra | N | R | 66.7 |
| Daucus carota | N | R | 21.1 | Kochia scoparia (L.) | N | O | 38.4 |
| | | | | Schrad. | | | |
| Diospiros Kaki | N | R | 100.0 | Krameria Triandra | N | R | 63.6 |
| Echinacea purpurea | N | O | 27.8 | Lentinus edodes | N | R | 100.0 |
| Eriobotrya japonica | N | R | 25.2 | Lentinus edodes | N | R | 26.2 |
| Eruca vesicaria | N | O | 34.5 | Ligularia dentata | N | O | 34.9 |
| Erysimum perofskianum | N | O | 91.0 | Ligustrum vulgare | N | O | 29.5 |
| Fish. S. | | | | | | | |
| Fragaria x ananassa | N | R | 37.5 | Lunaria annua | N | O | 72.3 |
| Fucus vesiculosis | N | R | 87.1 | Lunaria annua | N | R | 51.1 |
| Fumaria officinalis | N | O | 44.4 | Lupinus polyphyllus | N | O | 47.4 |
| | | | | lindl. | | | |
| Gaultheria procumbens | N | R | 74.8 | Lychnis chalcedonica | N | O | 34.4 |
| Gentiana macrophylla | N | O | 44.5 | Lythrum salicaire | N | R | 53.8 |
| Glyceria maxima | N | O | 37.6 | Mangifera indica | N | R | 100.0 |
| Glycine max Envy | N | O | 40.3 | Mangifera indica | N | O | 29.3 |
| Glycyrrhiza glabra | N | R | 37.7 | Nigella sativa | N | O | 26.1 |
| Hamamelis virginiana | N | R | 78.3 | Nil | N | O | 73.6 |
| Helichrysum | N | R | 21.8 | Nil | N | R | 25.4 |
| angustifolium | | | | | | | |
| Heliotropium | N | O | 26.8 | Nil | N | R | 24.6 |
| arborescens | | | | | | | |
| Humulus lupulus | N | R | 84.7 | Nil | N | R | 49.8 |
| Humulus lupulus | N | O | 39.2 | Nil | N | O | 43.6 |
| Nil | N | R | 28.4 | Salvia nemorosa | N | O | 38.2 |
| Optunia sp. | N | R | 100.0 | Sambucus canadensis | N | O | 27.5 |
| Panax quinquefolius L. | N | O | 27.4 | Sambucus nigra | N | O | 30.8 |
| Passiflora caerula | N | O | 39.8 | Sanguisorba minor | N | R | 78.3 |
| Pastinaca sativa | N | O | 20.5 | Saponaria officinalis | N | O | 68.7 |
| Perroselinum crispum | N | O | 60.9 | Saponaria officinalis L. | N | O | 44.2 |
| Phaseolus vulgaris | N | O | 37.5 | Satureja hortensis | N | O | 62.1 |
| Physostegia virginiana | N | O | 64.2 | Sechium edule | N | O | 34.4 |
| Phytolacca americana | N | O | 51.9 | Sesamum indicum | N | O | 78.6 |
| Phytolacca americana | N | O | 100.0 | Sidalcea | N | O | 42.9 |
| Plectranthus fruticosus | N | O | 23.4 | Silene vulgaris | N | O | 51.3 |
| Polygonatum odoratum | N | O | 100.0 | Solidago hybrida | N | O | 92.8 |
| Polygonium chinense | N | R | 33.6 | Solidago Hybrida | N | O | 100.0 |
| Pontederia cordata | N | O | 26.2 | Solidago Hybrida | N | R | 100.0 |
| Portulacea oleracea | N | O | 20.7 | Solidago sp. | N | O | 39.6 |
| Primula veris | N | O | 58.2 | Tamarindus indica | N | O | 64.2 |
| Prunus persica | N | R | 100.0 | Tanacetum balsamila | N | O | 100.0 |
| Prunus persica (hybride | N | R | 100.0 | Tanacetum vulgare | N | O | 23.3 |
| de la pêche) | | | | | | | |
| Pulmonaria officinalis | N | O | 22.8 | Taraxacum officinale | N | O | 90.9 |
| Punica granatum | N | R | 100.0 | Taraxacum officinale | N | O | 34.5 |
| | | | | (Red ribe) | | | |
| Pyrus pyrifolia | N | R | 22.4 | Thuja occidentalis | N | O | 37.6 |
| Radix Paeonia rubra | N | O | 39.8 | Thymus serpyllum | N | O | 20.6 |
| Rahmnus frangula | N | R | 25.3 | Tiarella | N | R | 35.6 |
| Raphanus sativus | N | O | 45.8 | Tragopogon sp. | N | R | 21.1 |
| Rhus trilobata | N | O | 20.2 | Trigonella foenum | N | R | 97.3 |
| | | | | graecum | | | |
| Ribes uva-crispa | N | R | 34.2 | Tropaeolum majus | N | O | 58.8 |
| Rosa Rugosa “Alba” | N | O | 45.4 | Tropaeolum majus | N | R | 28.6 |
| Rubus idaeus | N | R | 31.2 | Tropaeolum majus | N | O | 36.7 |
| Rubus idaeus L. | N | O | 42.7 | Tsuga diversifolia | N | R | 64.0 |
| Rubus ideaus | N | R | 74.2 | Vaccinium angustifolium | N | R | 72.2 |
| Rubus occidentalis | N | R | 68.1 | Vaccinium angustifolium | N | R | 50.7 |
| Rumex crispus Linné | N | R | 37.9 | Vaccinium macrocarpon | N | R | 52.6 |
| Vitia sp. | N | O | 35.1 | Weigela coracensis | N | R | 24.6 |
| Vitia sp. | N | R | 98.9 | Zea mays | N | R | 100.0 |
| Vitis sp. | N | R | 32.6 | Zea mays | N | R | 48.1 |

### Example III

### Exemplary Purification of Inhibitory Activity Found in an Extract

Extracts can be separated by HPLC on an Agilent 1100 system (San Fernando, Calif.). Briefly, 100 μL of a crude extract prepared as described in Example I can be applied on a C18 reverse-phase column (Purospher RP-18 5 μm, 4.0×125 mm (HP), Agilent, San Fernando, Calif.). Elution of compounds is achieved with a linear gradient of 10-85% acetonitrile. Fractions are collected, evaporated, resuspended in aqueous buffer and reanalysed for their inhibition activity on specific enzymes as already described. Fractions of interest (demonstrating a biological activity) can be reisolated at a larger scale for further analysis and characterisation.

### Example IV

### Preparation of Plant Extracts (Method B)

Method B is summarized in general terms in FIGS. 2 and 4. The method can be divided into two main parts corresponding to preliminary analytical scale extraction and a second larger scale extraction process.

### 1. Analytical Scale Extraction—Selection of Plants/Extracts

The processed plant materials (leaves, roots, or seeds) are obtained by dedicated greenhouse cultivation (with or without physical/chemical stress), from commercial suppliers, or by gathering from non-cultivated natural sources. For each plant used in either analytical scale or large scale extraction, a properly identified and labelled sample is kept in storage in the laboratory.

The extraction protocols for both the preliminary analytical scale and large scale extractions are shown generally in FIG. 4.

The collected dried plant material (2-10 g) is first submitted to solid-liquid extractions to generate crude extract A (mg scale). Two different solvents are tested (ethanol/methanol or ethanol/water mixtures). The extracts are then defatted with hexane to yield hydroalcoholic or alcoholic extract B and hexane extract C. A partitioning of extract B with ethyl acetate is then performed after dilution with water to yield aqueous extract E and organic extract F.

The extracts are sampled and evaluated for their ability to inhibit MMP-9 and/or Cathepsin B and their ability to inhibit endothelial or neoplastic cell migration using the methods described below.

Analysis of the results allows for the selection of plant materials for the large-scale extraction. The selection includes a decision regarding part of the plant and quantity of dried material needed to obtain sufficient mass of extract for pure active compound isolation. The selection also involves a choice of solvent system (aqueous versus alcoholic) and active extract (B, E or F) to be used in further work.

The extracts are also analyzed by Thin Layer Chromatography (TLC) with different reagents specific to classical chemical groups of natural products (terpenes, alkaloids, phenolic acids, polyphenols) to evaluate the increase in concentration achieved by partitioning at each step, and also to remove any materials likely to produce false positive results (fatty acids, chlorophylls) and to provide an indication of which fractionation steps to use in further extractions.

### 2. Large Scale Extraction—Isolation

For each new specimen, a repeat analytical scale extraction is performed to confirm the biological activity before beginning the large-scale extraction process.

The first step is to release the secondary metabolites from the dried and powdered material by means of an all purpose solvent mixture which is selected based on the results obtained in the analytical scale preparation. This can be done by successive maceration/percolation operations using the same solvent which should dissolve most natural compounds at the same time. The bulk of the inert and insoluble material such as cellulose is then removed by filtration. Conditions of drying and grinding are controlled (temperature of drying less than 45° C., particles size).

The second step is to remove a portion of the unwanted material in a series of liquid-liquid low resolution extractions using solvents of different polarity with the aim of a multi-gram mixture containing all the natural products of interest and to remove the most of the undesired material.

The extraction protocol is illustrated in FIG. 4 and is essentially the same as the procedure for the analytical preparation. The dried and pulverized material (2-3 Kg for large scale) is extracted repeatedly (maceration/percolation) with ethanol/methanol [85:15] v/v (a) or ethanol/water [85:15] v/v (b) mixtures (3×5-10 L) at room temperature for 2×24-48 h, based on the analytical scale results (yield of extraction).

In the case of an alcoholic extraction (a), the combined alcoholic extracts (A) are concentrated under reduced pressure, diluted with water (10-15%) and extracted with hexane (or heptane) to yield hexane extract (C) and hydroalcoholic fraction (B). This is then concentrated and diluted with ethanol (20%) before being extracted with ethyl acetate to yield aqueous (E) and ethyl acetate extracts (F).

In the case of a hydroalcoholic extraction (b), the combined aqueous extracts (A) are extracted with hexane to yield hexane extract (C) and hydroalcoholic fraction (B). The latter is then concentrated until residual water and diluted with ethanol (20%) before extraction with ethyl acetate to yield aqueous (E) and ethyl acetate extracts (F).

All the extracts (A-F) are sampled to verify the process recovery and the aliquots are submitted to a biological evaluation (MMP-9 and/or cathepsin B inhibition). The results are compared with those obtained on the analytical scale section and the selected positive extract is then concentrated to dryness under reduced pressure.

All the extracts are analyzed by TLC to compare with analytical scale extracts.

### Example V

### Effect of MMP-9 and Cathepsin B Inhibiting Plant Extracts on Cell Migration

Plant extracts were prepared as described in Example IV and underwent further testing to ascertain that they contain stable, non-cytotoxic molecules that are appropriate for product development. Stability is ascertained by recovery of protease inhibition over time under various conditions, including physiological conditions. Cytotoxicity is ascertained by incubation of the therapeutic combinations or components thereof with various cell types, including those indicated below.

The effects of the MMP-9 and cathepsin B inhibiting plant extracts on cellular migration cellular migration and/or cord formation were assessed as described below. Concentrations of plant extracts are expressed as a function of the IC₅₀ concentration determined for protease inhibition, which is termed 1×. The extracts are, therefore, capable of decreasing the activity of at least one extracellular protease by at least 50% when measured according to one of the assays described herein. The 1× concentration can vary depending on the plant and the solvent used in the preparation of the extract. The average concentration of a 1× aqueous extract is about 1.6 mg/ml, whereas the average concentration of a 1× alcoholic extract is about 4 mg/ml. For each extract tested in the assays described below, 4 different concentrations were used (0.31×, 0.62×, 1.25× and 2.5×) in duplicate.

### Cell Migration Assays

Migration was assessed using a multi-well system (Falcon 1185, 24-well format), separated by a PET membrane (8 μm pore size) into top and bottom sections. Depending on the cells that are used in the assay, the membrane was coated with 10 μg/ml rat tail collagen (for HUVECs) or with 80 μg/cm² of Matrigel growth factor (BD Biosciences) (for cancer cell lines) and allowed to dry. All solutions used in top sections were prepared in DMEM-0.1% BSA, whereas all solutions used in the bottom sections were DMEM, or other media, containing 10% fetal calf serum.

For HUVECs (Clonetics), EGM-2 (700 μl) was added to the bottom chamber as a chemo-attractant. HUVEC (100 μl of 10⁶ cells/ml) and buffer containing the plant extract at the appropriate dilution were added to the upper chamber (duplicate wells of each plant extract at each dilution). After 5 h incubation at 37° C. in a 5% CO₂ atmosphere, the membrane was rinsed with PBS, fixed and stained. The cells on the upper side of the membrane were wiped off, three randomly selected fields were counted on the bottom side.

The percent inhibition of migration is calculated as follows:

[(A−B)/A]×100,

where A is the average number of cells per field in the control well and B is the average number of cells per field in the treated wells.

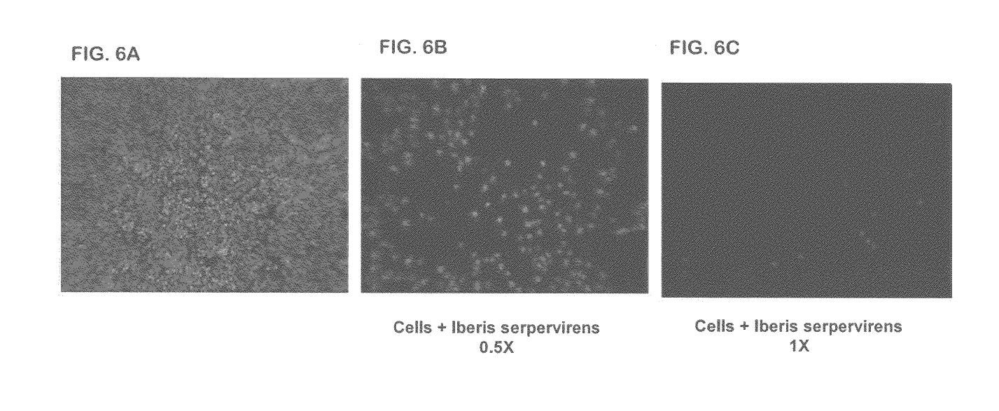

For cancer cell lines, prior to starting the experiment, the Matrigel impregnated filter was rehydrated with 200 μl of DMEM. A mixture of cells (100 μl of 2.5×10⁵/ml HT1080 or MDA-MB-231 cells, both from ATCC) and plant extracts were pipetted into the upper wells and 700 μl of DMEM-5% SVF was added to the bottom wells. The cells were incubated for 48 hours (HT1080 cells) or 72 hours (MDA-MB-231 cells), after which the membrane was treated as described above and inhibition of migration was determined as described above (see also FIG. 6, which shows the results using an extract from Iberis sempervirens).

### Cord Formation Assay

Matrigel (60 μl of 10 mg/ml) was added to a 96-well plate flat bottom plate (Costar 3096) and incubated for 30 minutes at 37° C. in a 5% CO₂ atmosphere. A mixture of HUVECs and plant extract, or positive controls (Fumagillin and GM6001) were added to each well. HUVECs were prepared as suspensions of 2.5×10⁵ cellsper ml in EGM-2, then 500 μl of HUVECs preparation was mixed with 5000 of 2× of the desired dilution of plant extract or control drug and 200 μl were added to each well. Four dilutions of each extract were tested in duplicate. After 18-24 hours at 37° C. in 5% CO₂, the cells had migrated and organized into cords.

The number of cell junctions were counted in 3 randomly selected fields and the inhibition of cord formation is calculated as follows:

[(A−B)/A]×100,

where A is the average number of cell junctions per field in the control well and B is the average number of cell junctions per field in the treated wells.

The results of the above experiments are presented in Tables 8 and 9. FIG. 6 shows cells treated with an extract from Iberis sempervirens.

TABLE 8
Effect of MMP-9 inhibiting plant extracts on endothelial cell migration

| | | | Endothelial Cell Migration | | | | | | | |
|---|---|---|---|---|---|---|---|---|---|---|
| | | | Cellular Migration Assay | | | | Cord Formation Assay | | | |
| | | | % inhibition | | | | % inhibition | | | |
| Plant | Stress¹ | Part of Plant² | 2.5 x | 1.25 x | 0.62 x | 0.31 x | 2.5 x | 1.25 x | 0.62 x | 0.31 x |
| Amaranthus candathus | G | L | 100 | 72 | 100 | 81 | 100 | 100 | 100 | 100 |
| Ambrosia artemisiifolia | N | Fl | 99 | 91 | 61 | 57 | 100 | 90 | 4 | 0 |
| Aronia x prunifolia | N | L/St | | 93 | 75 | 93 | 50 | 26 | 20 | 19 |
| Brassica napus | N | L | 51 | 33 | 0 | 0 | 77 | 59 | 43 | 41 |
| Brassica oleracea | N | L | 35 | 15 | 0 | 4 | 50 | 29 | 30 | 20 |
| Brassica oleracea | A | L | 49 | 28 | 27 | 6 | 65 | 32 | 15 | 21 |
| Bromus inermis | A | L | 21 | 14 | 0 | 93 | 90 | 44 | 36 | 17 |
| Chenopodium quinoa | N | L/St/Se | 90 | 85 | 53 | 42 | 100 | 100 | 44 | 26 |
| Citrullus lanatus | A | L | 21 | 17 | 6 | 0 | 88 | 35 | 23 | 14 |
| Dolichos lablab | G | Fl/Fr | 0 | 0 | 0 | 0 | 60 | 64 | 68 | 83 |
| Foeniculum vulgare | N | L | 69 | 21 | 23 | 11 | 64 | 47 | 62 | 61 |
| Hypomyces | N | Fr | 77 | 67 | 20 | 11 | 85 | 59 | 31 | 5 |
| lactifluorum | | | | | | | | | | |
| Lotus corniculatus | A | L/Fr/St | 9 | 0 | 0 | 0 | 93 | 83 | 77 | 57 |
| Lotus corniculatus | N | Se | 0 | 0 | 0 | 0 | 58 | 11 | 26 | 0 |
| Manihot esculenta | N | Fr | 39 | 0 | 0 | 0 | 33 | 30 | 25 | 26 |
| Matricaria recutita | G | L/Fl/St | 34 | 31 | 4 | 0 | 74 | 6 | 1 | 20 |
| Melilotus albus | G | L/St | | | | 0 | 70 | 15 | 0 | 0 |
| Phaseolus vulgaris | A | L | 51 | 17 | 4 | 7 | 54 | 29 | 10 | 18 |
| Phaseolus vulgaris | G | L | 33 | 13 | 25 | 18 | 82 | 56 | 51 | 41 |
| Pisum sativum | N | L/St | 16 | 24 | 4 | 0 | 38 | 16 | 13 | 0 |
| Raphanus raphanistrum | G | L | 46 | 24 | 10 | 0 | 88 | 46 | 23 | 23 |
| Ribes sylvestre | N | L | 96 | 87 | 56 | 26 | 59 | 49 | 69 | 56 |
| Rumex crispus | A | R | 96 | 83 | 0 | 18 | 96 | 46 | 17 | 13 |
| Rumex crispus | G | R | 36 | 0 | 36 | 0 | 80 | 100 | 86 | 36 |
| Rumex scutatus | N | L | 70 | 6 | 0 | 0 | 100 | 20 | 0 | 0 |
| Tanacetum | G | L | 100 | 99 | 56 | 0 | 100 | 100 | 42 | 18 |
| cinerariifolium | | | | | | | | | | |
| Tropaeolum majus | G | L | 7 | 0 | 0 | 0 | 65 | 29 | 18 | 4 |
| Tsuga canadensis | N | L/Fr/St | | 80 | 82 | 64 | 68 | 41 | 31 | 31 |
| Tsuga diversifolia | N | L/St | 57 | 8 | 0 | 0 | 99 | 43 | 18 | 27 |
| Vaccinium | N | Fr | 59 | 15 | 6 | 0 | 62 | 7 | 11 | 24 |
| angustifolium | | | | | | | | | | |
| Zea mays | N | L | 11 | 0 | 0 | 11 | 66 | 24 | 14 | 6 |
| Zingiber officinale | N | Fr | 0 | 0 | 0 | 0 | 59 | 38 | 27 | 30 |

¹A: Arachidonic Acid; G: Gamma-Linolenic Acid; N: No stress treatment
²EP: Entire plant; Fl: Flower; Fr: Fruit; L: Leaf, R: Root; Se: Seed; St: Stem

TABLE 9
Effect of cathepsin B inhibiting plant extracts on neoplastic cell
migration

| | | | Migration of Cancer Cells | | | |
|---|---|---|---|---|---|---|
| | | | % inhibition | | | |
| Plant | Stress¹ | Part of plant² | 2.5 x | 1.25 x | 0.62 x | 0.31 x |
| Allium tuberosum | G | Fr/Fl | 68 | 0 | 0 | 0 |
| Allium tuberosum | A | Fr/Fl | 73 | 76 | 80 | 36 |
| Althacea officinalis | N | L/St | 66 | 0 | 0 | 0 |
| Ambrosia artemisiifolia | N | Fl | 92 | 76 | 0 | 0 |
| Angelica sinensis | N | EP | 100 | 75 | 32 | 53 |
| Aronia x prunifolia | N | L/St | 95 | 94 | 95 | 97 |
| Asarum europaeum | G | L | 67 | 49 | 0 | 73 |
| Begonia Hannii | A | L/Fl/Fr/St | 100 | 100 | 14 | 0 |
| Begonia polygonoides | A | L/Fl/St | 100 | 0 | 0 | 0 |
| Brassica oleracea | N | L | 78 | 45 | 49 | 57 |
| Bromus inermis | A | L | 91 | 91 | 93 | 90 |
| Chenopodium quinoa | N | L/St/Se | 100 | 99 | 58 | 31 |
| Conyza canadensis | G | EP | 65 | 8 | 0 | 0 |
| Cynara cardunculus | G | Fr | 99 | 39 | 33 | 48 |
| subsp. Cardunculus | | | | | | |
| Daucus carota | G | L | 0 | 30 | 0 | 38 |
| Hypomyces | N | Fr | 66 | 72 | 0 | 0 |
| lactifluorum | | | | | | |
| Iberis sempervirens | A | L/St | 100 | 42 | 4 | 0 |
| Iberis sempervirens | G | L/St | 100 | 100 | 98 | 91 |
| Lunaria annua | N | Fr | 100 | 100 | 68 | 9 |
| Melilotus albus | G | L/St | 54 | 0 | 0 | 0 |
| Phaseolus vulgaris | G | L | 43 | 2 | 0 | 0 |
| Physostegia virginiana | G | L/St | 78 | 0 | 0 | 0 |
| Pisum sativum | N | L/St | 27 | 23 | 12 | 9 |
| Ribes sylvestre | N | L | 91 | 87 | 17 | 0 |
| Rubus occidentalis | N | Fr | 84 | 82 | 89 | 90 |
| Rumex crispus | A | R | 96 | 89 | 8 | 0 |
| Rumex crispus | G | R | 99 | 86 | 0 | 0 |
| Salvia officinalis | A | L/St | | 98 | 89 | 39 |
| Solidago canadensis | G | Fl | 100 | 100 | 93 | 93 |
| Solidago sp. | A | L/Fl/St | 100 | 83 | 0 | 0 |
| Solidago x hybrida | N | L/St | 100 | 96 | 70 | 7 |
| Solidago x hybrida | A | L/St | 100 | 90 | 0 | 0 |
| Solidago x hybrida | N | Fl | 100 | 51 | 13 | 0 |
| Solidago x hybrida | A | Fl | 100 | 99 | 91 | 89 |
| Taraxacum officinale | N | L | 100 | 71 | 47 | 0 |
| Tsuga canadensis | N | L/St | 65 | 64 | 63 | 0 |
| Tsuga diversifolia | N | L/St | 100 | 63 | 38 | 90 |
| Zea mays | N | L | 36 | 35 | 25 | 24 |
| Zingiber officinale | N | R | 90 | 56 | 13 | 0 |

¹A: Arachidonic Acid; G: Gamma-Linolenic Acid; N: No stress treatment
²EP: Entire plant; Fl: Flower; Fr: Fruit; L: Leaf; R: Root; Se: Seed; St: Stem

### Example VI

### Effect of Plant Fraction Compositions on Human Protease Activity

The following plant extracts were prepared from unstressed plants according to the method outlined in Example IV. Briefly, a solid-liquid extraction using ethanol/water was conducted to generate a crude extract, which was subsequently defatted with hexane to yield the hydroalcoholic plant extract.

Plant extract A: a Solidago sp. leaf/flower/stem extract that inhibits cathepsin B*. * The Solidago sp. extract was derived from plants harvested in Quebec, Canada, and as such can contain Solidago canadensis, Solidago gigantea, Solidago hybrida, or a combination thereof. An extract derived from Solidago virgaurea obtained from a commercial source gave similar results.
Plant extract B: a Zingiber officinale root extract that inhibits MMP-9.

### Enzymes

Human MMP-9 was purified from natural sources (THP-1 cell line ATCC, Mannassas, Va., USA) as described in the literature (Shimokawa K, Nagase H. Methods Mol Biol. 2001; 151:275-304). Human cathepsin B (from liver) was purchased from Calbiochem (San Diego, Calif., USA).

### Assay

MMP-9 proteolytic activity was assayed by cleavage of an auto-quenched peptide substrate (MCA-Pro-Leu-Gly-Leu-Dpa-Ala-Arg-NH₂) in assay buffer (20 mM Tris-HCl; NaCl 150 mM; CaCL₂ 5 mM; ZnCl₂ 0.5 mM; pH 7.5) according to Shimokawa K, Nagase H. Methods Mol Biol. 2001; 151:275-304.

Cathepsin B proteolytic activity was assayed by cleavage of an auto-quenched peptide substrate (Z-Arg-Arg-AMC) according to Barrett A J, Kirschke H. Cathepsin B, Cathepsin H, and cathepsin L. Methods Enzymol. 1981:535-61. All substrates were supplied by Calbiochem (San Diego, Calif., USA).

Fluorescence kinetic measurements were performed on a Polarion fluorometer (Tecan). All analyses were performed in duplicate and met quality control criteria (experimental error <10%). Fluorescence measurements with the enzyme should be three times higher than noise level. Fixed concentrations of positive controls (GM-6001 for MMP-9 and CA-074 for cathepsin B) were used as inter-assay controls. A negative control (buffer+substrate for both enzymes) was also included in order to determine the noise level.

Enzyme inhibition by the tested plant extracts was calculated by comparing the enzyme activity with and without plant extract. IC₅₀ values refer to the plant extract concentration that inhibits the activity of the target enzyme by 50%. Results are shown in Table 10.

TABLE 10
IC₅₀ values for Plant Extracts A and B

| Plant Extract | Target | IC₅₀ (μg/mL) |
|---|---|---|
| A | Cath B | 220 |
| B | MMP-9 | 25 |

### Example VII

### In Vitro Cytotoxicity Assays

The cytotoxicity of plant extracts A and B (see Example VI) on various cell lines were evaluated according to Pagé, B., et al., Int. J. Oncol. 3, 473-476 (1993). In brief, cells were plated at 2×10³ (HUVEC, PC-3, HT1080, L929, B16F10, LLC/M27), at 5×10³ (MDA, MRCS) or at 10×10³ (Caco-2 and HepG2) per well and after 24 hours the appropriate plant extract was added and the cells were incubated for an additional 72 hours at 5% CO₂, 37° C. Various concentrations of the plant extracts were tested ranging from 0.012 to 0.4 mg/mL. The survival of cells was evaluated using the Alamar Blue assay. The results are shown in Table 11, the concentration provided in the Table represents the amount of each extract that resulted in 50% cell death.

TABLE 11
In vitro cytotoxicity

| | | Plant fraction cytotoxic | |
|---|---|---|---|
| | | concentration (mg/mL) | |
| Cell line | Origin | Plant Extract A | Plant Extract B |
| LLC/M27 | Murine lung carcinoma | >0.1 | >0.1 |
| B16F10 | Murine melanoma | >0.05 | >0.1 |
| L929 | Murine fibrosarcoma | >0.1 | >0.1 |
| CaCO2 | Human colon carcinoma | >0.12 | >0.1 |
| HT1080 | Human fibrosarcoma | Not determined | >0.1 |
| MRC5 | Human fetal lung | >0.1 | >0.1 |
| | fibroblast | | |
| HepG2 | Human liver cancer | Not determined | >0.1 |
| HUVEC | Human umbilical vein | Not determined | >0.1 |
| | endothelial cells | | |
| PC3 | Human prostate cancer | >0.32 | Not determined |
| MDA- | Human breast cancer | >0.2 | Not determined |
| MB231 | | | |

### Example VIII

### Effect of Plant Extracts in HUVEC Cord Formation Assays

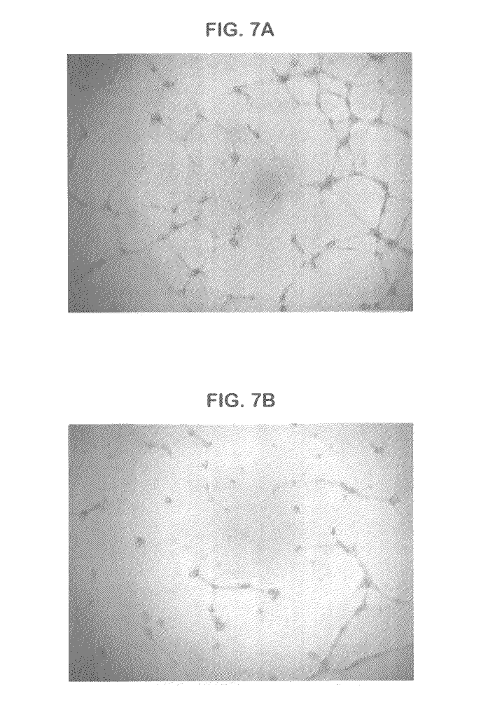

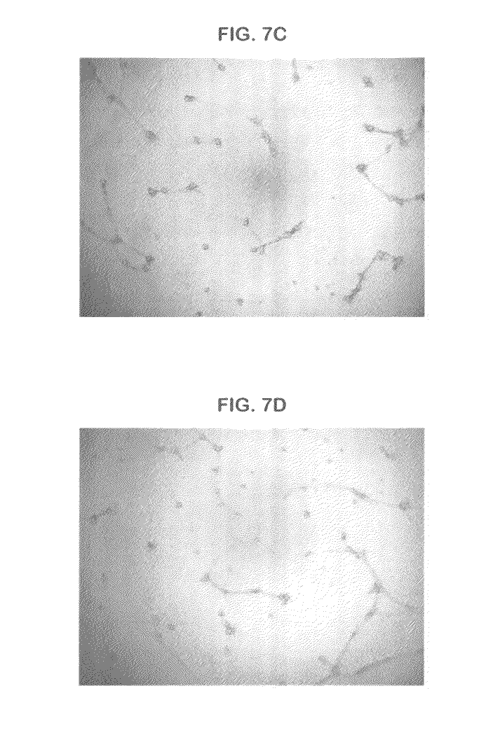

Plant extract B (see Example VI) was tested in a HUVEC cord formation assay performed according to the National Cancer Institute protocol. Briefly, human umbilical vein endothelial cells (HUVEC, Cambrex, Walkersville, Md.) were seeded at fourth passage (25×10⁴ cells per well) in HUVEC complete medium (EGM-2®) on Matrigel® (Becton Dickinson, Franklin Lakes, N.J.). Plant extract or controls were added in 100 μl ml of EGM-2 per well and cells were incubated 18 hours at 37° C., 5% CO₂. Plates were then examined by light microscopy for qualitative and quantitative analysis of the three dimensional capillary-like structures formed by the endothelial cells on the Matrigel® matrix. GM-6001 (an MMP inhibitor) and Fumagilin (an angiogenesis inhibitor as per NIH protocol) were used as positive controls. Representative results are shown in FIG. 7. A. negative control (vehicle); B. positive control GM-6001 (25 μg/mL); C. positive control Fumagilin (15 μg/mL), and D. plant extract B (10 μg/mL).

### Example IX

### Effect of Plant Extracts in Tumour Cell Invasion Assays

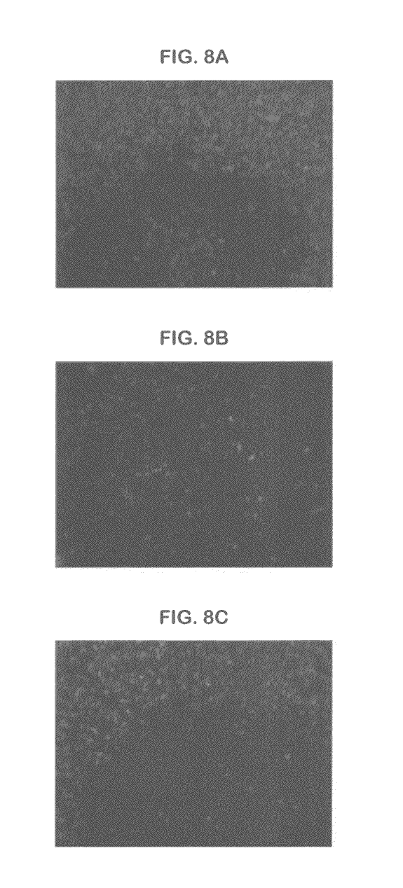

Plant extract A (see Example VI) was tested in a tumour cell invasion assay as follows. MDA-MD231 breast adenocarcinoma cells (ATCC HTB-26) were seeded at 25×10⁴ cells per well on a thin Matrigel® coating of 120₄ g/cm² applied on a 8μ-porous membrane of 96-well MultiscreenMIC plates (Millipore). The cells were seeded in the upper compartment and incubated in the presence of controls or plant extract in DMEM-0.1% BSA media. A chemoattractant, DMEM media with 10% FCS (Fetal calf serum, Wisent), was loaded in the lower compartment. Cells treated with the plant extract were incubated for 48 hours at 37° C., 5% CO₂. All media were then removed and the cells that had migrated to the lower compartment were fixed and stained with propidium iodine whereas the cells remaining in the upper compartment were removed. The invasive cells were examined under inverted fluorescent microscope and counted using ImagePro Plus software (Carsen Group, Markham, Ontario, Canada). Non-invasive MCF7 cells (breast adenocarcinoma, ATCC HTB-22) were used as a control. Representative results are shown in FIG. 8. A. invasive cells (MDA-MD231); B. non-invasive cells (MCF7); and C. plant extract A (50 μg/mL).

### Example X

### In Vivo Toxicity of Plant Extracts and Plant Extract Compositions

The oral toxicity of plant extracts A and B (see Example VI) in single and multiple doses, separately and in combination (1:1 ratio) was evaluated in fasted (2 hrs) C57BL/6 mice (n=6). The results are shown in Table 12. No effect body weight was observed during the period of investigation.

TABLE 12
In vivo Toxicology Results

| | | Dose | |
|---|---|---|---|
| Duration | Fraction | (mg/kg) | Observations |
| Single dose | Vehicle control | 0 | No clinical or gross necropsy |
| | | | observations |
| | Plant extract B | 400 | No clinical or gross necropsy |
| | | | observations |
| | Plant extract A | 400 | No clinical or gross necropsy |
| | | | observations |
| 7 day | Vehicle control | 0 | No clinical or gross necropsy |
| | | | observations |
| 7 day | Plant extract A + | 200/200 | No clinical or gross necropsy |
| (com- | plant extract B | | observations |
| bination) | | | |

### Example XI

### Anti-Metastatic Effect of Plant Extract Compositions on Tumour Metastasis

The objective of this example was to evaluate the anti-metastatic activity of plant extract compositions alone or in combination with a chemotherapeutic in the Lewis lung carcinoma (LLC) model of tumour metastasis in the mouse. As shown in Example X, administration of these plant extracts individually or in combination has been shown to be non-toxic when administered orally for 7 consecutive days to C57BL/6 mice.

The Lewis lung carcinoma model in C57BL/6 mice was used for this study. Lewis lung carcinoma is an aggressive, highly metastatic cell line. LLC1 cells clone M27 (3×10⁵ cells, screened for mycoplasma) were injected on Day 0 into the tail vein of each mouse. The mice were divided into 7 groups and received the treatments outlined below.

Plant extracts A and B as described in Example VI together with the following plant extract were used in this study:

Plant extract C: a Tsuga canadensis leaf/stem extract that inhibits MMP-9.

Plant extract C was prepared from unstressed plants according to the method outlined in Example IV.

Oral administration of plant extracts was initiated 9 days prior to injection of LLC cells (i.e. on day −9) and continued for 14 consecutive days along with sub-optimal doses of cisplatin (see below).

Group 1: Hydroxypropyl-beta-cyclodextrin (30%), the vehicle used for plant extracts was used as a negative control for the experiment.

Group 2: Cisplatin (5 mg/kg), a standard positive control in the Lewis lung carcinoma model was'injected intraperitoneally on days 1, 4, 7, 10 and 13.

Group 3: Cisplatin (2 mg/kg), a sub-optimal dose in this model, was injected intraperitoneally on days 1, 4, 7, 10 and 13.

Group 4: Therapeutic combination 1 (TC1) was administered to this group. TC1 comprised plant extract A, plant extract B and cisplatin (2 mg/kg). Plant extracts A and B were administered by gavage (200 mg/kg of each extract) from days −9 to 14 and cisplatin was injected intraperitoneally on days 1, 4, 7, 10 and 13.

Group 5: Therapeutic combination 2 (TC2) was administered to this group. TC2 comprised plant extract A, plant extract C and cisplatin (2 mg/kg). Plant extracts A and C were administered by gavage (200 mg/kg of each extract) from days −9 to 14 and cisplatin was injected intraperitoneally on days 1, 4, 7, 10 and 13.

Group 6: Therapeutic combination 3 (TC3) was administered to this group. TC3 comprised plant extract B and cisplatin (2 mg/kg). Plant extract B was administered by gavage (200 mg/kg) from days −9 to 14 and cisplatin was injected intraperitoneally on days 1, 4, 7, 10 and 13.

Group 7: Therapeutic combination 4 (TC4) was administered to this group. TC4 comprised plant extract C and cisplatin (2 mg/kg). Plant extract C was administered by gavage (200 mg/kg) from days −9 to 14 and cisplatin was injected intraperitoneally on days 1, 4, 7, 10 and 13.

At the end of the experiment (Day 14), the animals were humanely sacrificed, the lungs resected and fixed by direct immersion for approximately 24 hrs in Bouin's fixing media. Metastatic colonies on each lung surface of each mouse were counted by direct observation under a dissecting microscope in a blinded manner by three different investigators.

The experiment was considered valid as the following criteria were met:
1) The number of lung tumours in control animals was sufficient to compare to treated groups.
2) The number of lung tumours in the 5 mg/kg cisplatin group (Group 2) was statistically significantly lower than in the control Group 1.
3) The number of lung tumours in cisplatin 2 mg/kg group (Group 3) was higher in a statistically significant manner than the cisplatin 5 mg/kg group (Group 2).

One mouse had to be sacrificed on Day 12 due to deteriorating condition (Group 1). The results of the experiment are shown in Table 13.

TABLE 13
Tumour count (mean values of three independent counts)

| | Group | | | | | | |
|---|---|---|---|---|---|---|---|
| | | 2 | 3 | | | | |
| | 1 | Cisplatin | Cisplatin | 4* | 5* | 6* | 7* |
| | Untreated | 5 mg | 2 mg | TC1 | TC2 | TC3 | TC4 |
| Mean (SD) | 33.69 | 1.46 | 24.70 | 11.14 | 16.72 | 16.24 | 15.04 |
| tumours/animal | (35.71) | (3.95) | (36.65) | (26.73) | (30.01) | (27.25) | (30.63) |
| Total tumour | 472 | 21 | 346 | 156 | 217 | 227 | 211 |
| burden | | | | | | | |
| (per group) | | | | | | | |
| % reduction | 0 | 95.6 | 26.7 | 66.2 | 50.4 | 51.8 | 56.4 |
| compared to | | | | | | | |
| control | | | | | | | |

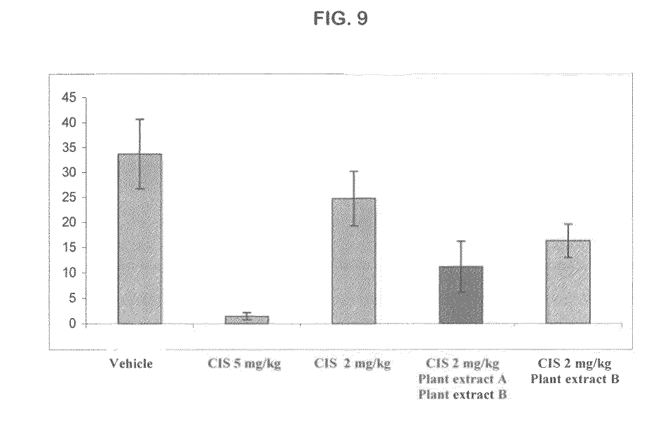

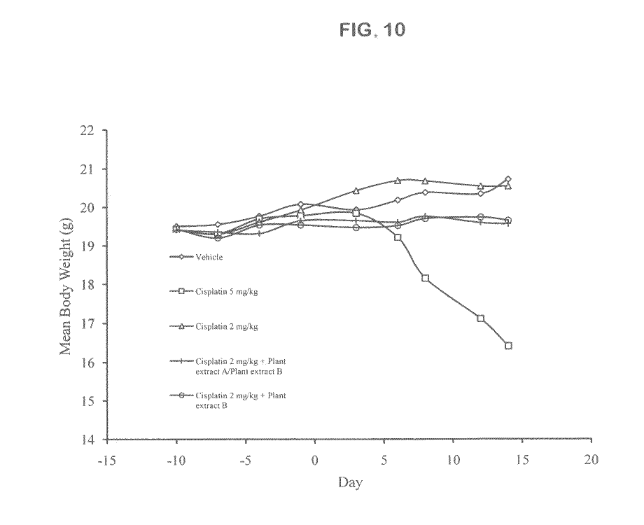

The above results show that:
1) Cisplatin at the 5 mg/kg dose reduced the number of lung tumour metastases in this model (96% reduction, p=<0.001).
2) Cisplatin at the 2 mg/kg dose only marginally reduced the number of lung tumour metastases in this model (27% reduction, p=0.02).
3) TC1 reduced the number of lung tumour metastases in this model (66%) in a statistically significant manner compared to cisplatin 2 mg/kg alone (p=0.015), as shown in FIG. 9 which demonstrates that TC1 induced statistically significant (p<0.05) inhibition of metastatic expansion compare to cisplatin (2 mg/kg) alone and vehicle (Group 1).
4) TC2, TC3, and TC4 reduced lung tumour metastasis in this model with values of 50, 52 and 56% respectively.
5) Body weights of animals treated with plant extracts A and B remained stable throughout the experiment, as shown in FIG. 10. In contrast, a sharp decrease in body weight was observed for the animals treated with cisplatin at the 5 mg/kg dose (consistent with previous observations).

The number of lung tumour metastases was significantly lower in animals from Group 4, treated with TC1, when compared to those in Group 6, treated with TC3 (p=0.033) suggesting that extract A acts in synergy with extract B in this model.

### Example XII

### Effect of Plant Extract Compositions on Tumour Growth

The objective of this example was to evaluate the activity of plant extract compositions alone or in combination with a chemotherapeutic agent in the B16F10 melanoma model of tumour growth in the mouse. As shown in Example X, administration of these plant extracts individually or in combination has been shown to be non-toxic when administered orally for 7 consecutive days to C57BL/6 mice.

The B16F10 melanoma model in C57BL/6 mice was used for this study. B16F10 cells (1×10⁶ cells, screened for mycoplasma) were injected subcuteanously on Day 0 on the right flank of each mouse. The mice were divided into 9 groups and received the treatments outlined below. Plant extracts A and B (see Example VI) were used in this study.

Oral administration of plant extracts was initiated 7 days prior to injection of the B16F10 cells (i.e. on day −7) and continued for 14 consecutive days with or without sub-optimal doses of doxorubicin (see below).

Group 1: The vehicle used for administration of the plant extracts was used as a negative control for the experiment.

Group 2: Doxorubicin (2.5 mg/kg), a standard positive control in the B16F10 melanoma model at optimal dosage, was injected intraperitoneally on days 5, 9 and 13.

Group 3: Doxorubicin (1 mg/kg), a sub-optimal dose in this model, was injected intraperitoneally on days 5, 9 and 13.

Group 4: Therapeutic plant extract A (PA1) was administered by gavage (200 mg/kg) from days −7 to 14.

Group 5: Therapeutic plant extract B (PB1) was administered by gavage (200 mg/kg) from days −7 to 14.

Group 6: Therapeutic combination 5 (TC5) was administered to this group. TC5 comprised plant extract A and doxorubicin (1 mg/kg). Plant extract A was administered by gavage (200 mg/kg) from days −7 to 14 and doxorubicin was injected intraperitoneally on days 5, 9 and 13.

Group 7: Therapeutic combination 6 (TC6) was administered to this group. TC6 comprised plant extract B and doxorubicin (1 mg/kg). Plant extract B was administered by gavage (200 mg/kg) from days −7 to 14 and doxorubicin was injected intraperitoneally on days 5, 9 and 13.

Group 8: Therapeutic combination 7 (TC7) was administered to this group. TC7 comprised plant extract A, plant extract B and doxorubicin (1 mg/kg). Plant extracts A and B were administered by gavage (200 mg/kg of each extract) from days −7 to 14 and doxorubicin was injected intraperitoneally on days 5, 9 and 13.

Group 9: Therapeutic combination 8 (TC8) was administered to this group. TC8 comprised plant extract A and plant extract B. Plant extracts A and B were administered by gavage (200 mg/kg of each extract) from days −7 to 14.

The subcutaneous tumour was measured on each animal with an electronic calliper starting on day 5 and repeated on days 8, 11 and 14 and the volume of tumour was calculated according to formula: L×1²×0.53. At the end of the experiment, the animals were sacrificed.

TABLE 13
Tumour data on Day 14 expressed as tumour volume, percentage
growth and tumour diameter

| | Group | | | | | | | | |
|---|---|---|---|---|---|---|---|---|---|
| | | | | | | | 7 | | 9 |
| | | | | | | 6 | PB1/ | 8 | PA1/ |
| | 1 | 2 | 3 | 4 | 5 | PA1/Doxo* | Doxo* | PA1/PB1/Doxo* | PB1 |
| | Control | Doxo* | Doxo* | PA1 | PB1 | (TC5) | (TC6) | (TC7) | (TC8) |
| Doses | Vehicle | 2.5 | 1 | 200 | 200 | 200/1 | 200/1 | 200/200/1 | 200/200 |
| Volume (mm³) | 2375 ± 381 | 1563 ± 168 | 2682 ± 220 | 2842 ± 362 | 3380 ± 467 | 2641 ± 456 | 2236 ± 220 | 1505 ± 253 | 2421 ± 254 |
| % growth | 228 ± 19 | 196 ± 34 | 247 ± 30 | 277 ± 27 | 205 ± 19 | 184 ± 22 | 189 ± 16 | 159 ± 35 | 230 ± 26 |
| Diameter (mm) | 20.1 ± 1.2 | 20.2 ± 0.9 | 21.4 ± 1.1 | 22.5 ± 1.3 | 24.2 ± 1.4 | 20.0 ± 1.2 | 20.2 ± 1.1 | 17.4 ± 1.2 | 19.9 ± 0.6 |

*Doxo: doxorubicin

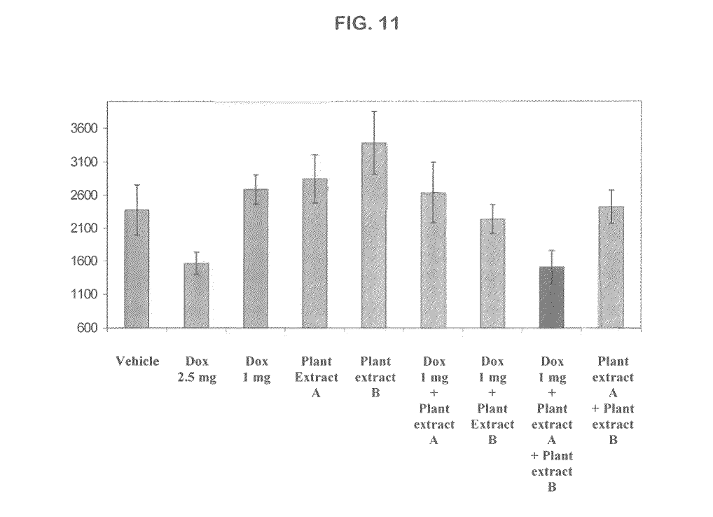

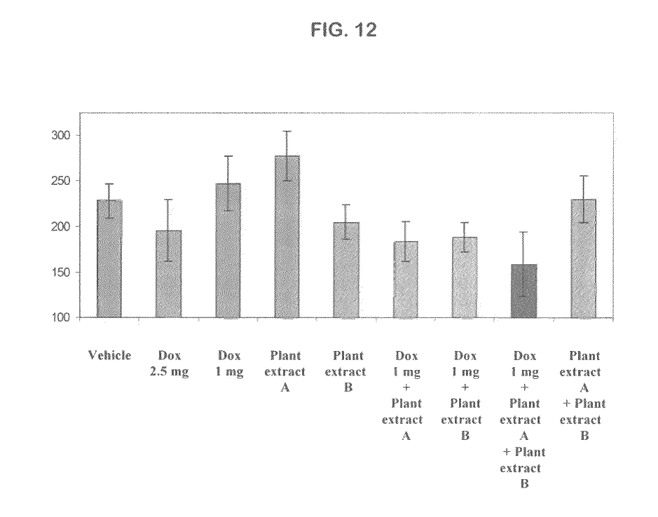

The above results show that:
1) The treatment with TC7 was as effective at reducing tumour diameter and volume as the therapeutic dose (2.5 mg/mL) of doxorubicin compared to the sub-optimal dose of doxorubicin (1 mg/kg) and the control (p<0.05). See FIG. 11.
2) The combination of PA1 and PB1 potentiates the effect of sub-optimal dose treatment of doxorubicin (1 mg/kg) compared to this dose of doxorubin (1 mg/kg) alone (44% tumour volume reduction, p<0.05). See FIG. 12.

### Example XIII

### Formulation of Plant Extracts

The following is an exemplary therapeutic formulation of the present invention. The formulation comprises two plant extracts and may be administered in the form of gel caps, a powder or a predose pouch, alone or in combination with one or more chemotherapeutic agents. The specific formulation described below is prepared as a 10 g single dose pouch, which is dissolved in water prior to administration. The formulation is intended for oral administration.

Formulation for a 10 g single dose pouch:
At least 2 g of lecithin
1-3 g of Zingiber officinale extract^{†}^{†}Zingiber officinale extract was prepared from dried rhizome using 50% ethanol in water as solvent.
1-3 g of Solidago sp. extract* * Solidago sp. extract was derived from Solidago sp. Ph. Eur. and thus contains Solidago canadensis L. and/or Solidago gigantea Ait. The extract was prepared from the dried aerial parts of the plants using 60% ethanol in water as solvent.
Silica dioxide to prevent agglomeration

### Sweetener

### Example XIV

### Demonstration of Dose-Dependent Effect in Preventing Metastases of LLC in a Mouse Model

The following is an exemplary method of determining a dose-dependent effect of the plant extracts alone or in combination with a chemotherapeutic in preventing metastasis in the LLC mouse model. The experimental protocol described in Example XI and the experimental design outlined in Table 14 can be used.

TABLE 14
Exemplary Experimental design

| | Suggested Dose | | Suggested | Approximate |
|---|---|---|---|---|
| Treatment | (mg/kg) | | Duration | No. of animals |
| Vehicle | 0 | mg/kg | 14 Days | 12 |
| Cisplatin | 5 | mg/kg | 14 Days | 12 |
| Cisplatin | 2 | mg/kg | 14 Days | 12 |
| Negative control plant | 200 | mg/kg | 14 days | 12 |
| extract | | | | |
| MMP-9 inhibitor | 200 | mg/kg | 14 Days | 12 |
| Cathepsin B inhibitor | 200 | mg/kg | 14 Days | 12 |
| MMP-9 inhibitor/ | 200/200 | mg/kg | 14 Days | 12 |
| Cathepsin B inhibitor | | | | |
| MMP-9 inhibitor/ | 300/300 | mg/kg | 14 Days | 12 |
| Cathepsin B inhibitor | | | | |
| Cisplatin/MMP-9 | 2/200/200 | mg/kg | 14 Days | 12 |
| inhibitor/Cathepsin B | | | | |
| inhibitor | | | | |

Examples of parameters indicative of a positive outcome for this experiment are:
1. Statistically significant differences (p value <0.05) in number of metastases between inhibitor treated groups and negative control.
2. Statistically significant differences (p value <0.05) in number of metastases between inhibitor+cisplatin treated groups and cisplatin alone.

3. Statistically significant differences (p value <0.05) in number of metastases between combination of inhibitors and either inhibitor alone.

A positive outcome in the three above-defined parameters would prove efficacy as a single therapy, improved efficacy when combined with first-line standard chemotherapy and/or positive synergism of both inhibitors.

### Example XV

### Determining the Efficacy of the Plant Extracts in Mouse Xenograft Models

The following is an exemplary protocol for testing the activity of the plant extracts alone or in combination with a chemotherapeutic in a mouse xenograft assay using human cancer cell lines.

In this model, human tumour cells are transferred to an immuno-compromised mouse, most often subcutaneously because of the ease of injection and subsequent tumour evaluation. Tumours usually require a few days to a few months to grow, depending on the growth rate and the cell line used. Examples of human tumour xenografts that can be used in these experiments include breast, colon, prostate, melanoma and lung tumours.

A proposed experimental design utilising xenograft models is shown in Table 15:

TABLE 15
Exemplary experimental design for mouse xenograft model

| | | | | Approximate |
|---|---|---|---|---|
| | Suggested Dose | | Suggested | No. of |
| Treatment | (mg/kg) | | Duration* | animals |
| Vehicle | 0 | mg/kg | 21 Days | 12 |
| Positive control | # | | 21 Days | 12 |
| Positive control (lower | # | | 21 days | 12 |
| dose) | | | | |
| Negative control plant | 200 | mg/kg | 21 days | 12 |
| extract | | | | |
| MMP-9 inhibitor | 200 | mg/kg | 21 Days | 12 |
| Cathepsin B inhibitor | 200 | mg/kg | 21 Days | 12 |
| MMP-9 inhibitor/ | 200/200 | mg/kg | 21 Days | 12 |
| Cathepsin B inhibitor | | | | |
| Positive control/MMP-9 | #/200/200 | mg/kg | 21 Days | 12 |
| inhibitor/Cathepsin B | | | | |
| inhibitor | | | | |

*Duration of administration may vary depending on cell line selected.
# Dose of the positive control will be dependent on the drug selected.

The positive control chemotherapeutic used in this study should be one that has been shown to be effective with the specific cancer cell line selected. Examples of cancer cell lines and chemotherapeutics that could be used are human prostate adenocarcinoma cells (PC-3) and cisplatin, and human colorectal adenocarcinoma cells (HT-29) and vincristine. In brief, the selected cells are injected subcutaneously into female NU/NU-nuBR mice and the length (L) and width (W) of resulting tumours are measured in millimiters using vernier calipers. Tumour weights are calculated by using the following formula: mg=(L×W²)/2.

Once the potency of each plant extract component of the therapeutic combination is established separately, studies with a combination of extracts inhibiting MMP-9 and/or cathepsin B can be conducted to determine the most efficacious ratio of each extract within the therapeutic combination.

Examples of parameters indicative of a positive outcome are:
1) Statistically significant differences (p value <0.05) in the mean size of tumours between therapeutic composition treated groups and negative control.
2) Statistically significant differences (p value <0.05) in the mean size of tumours between therapeutic composition and positive control alone.
3) Statistically significant differences (p value <0.05) in the mean size of tumours between a therapeutic combination and individual components of the therapeutic combination alone.

### Example XVI

### Determining the Efficacy of the Plant Extracts in Mouse Orthotopic Xenograft Models

The following is an exemplary protocol for testing the activity of the plant extracts alone or in combination with a chemotherapeutic in a mouse orthotopic xenograft assay using human cancer cell lines.

A recently developed technique using green fluorescent protein (GFP) expressing tumours and non-invasive whole-body imaging can be used (Yang et al, Proc. Nat. Aca. Sci, February 2000, pp 1206-1211). In this model, human or murine tumours that stably express very high levels of the Aqueora vittoria green fluorescent protein can be transplanted orthotopically into nude mice. The GFP expressing tumours can be visualized by means of externally placed video detectors, allowing for monitoring of details of tumour growth, angiogenesis and metastatic spread. Angiogenesis can be measured over time by monitoring the blood vessel density within the tumour(s).

Overall, the study design for the orthotopic xenograft study will be similar to the one used for subcutaneous tumour growth as outlined in Table 15. The cancer types used can include, for example, human colon (HT-29) or prostate (PC-3) cancer cells that are injected into the colon or prostate, respectively, of nude mice. The positive control chemotherapeutic used in this study should be one that has been shown to be effective with the specific cell line used. Again, the potency of each plant extract in the composition, as well as the therapeutic combinations, can be established separately.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1.-53. (canceled)

54. A composition for inhibition of MMP-9 and cathepsin B activity, said composition comprising two or more plant extracts, each of said plant extracts capable of inhibiting MMP-9 and/or cathepsin B activity, and a physiologically acceptable carrier, wherein said composition inhibits: (a) MMP-9 and cathepsin B activity, and (b) one or more of neoplastic cell migration, endothelial cell migration, tumour growth, tumour metastasis, and tumour-induced angiogenesis.

55. The composition according to claim 54, wherein at least one of said plant extracts is derived from a plant belonging to the Zingiber genus of plants, the Tsuga genus of plants or the Solidago genus of plants.

56. The composition according to claim 54, wherein at least one of said plant extracts is derived from a plant selected from the group of Zingiber officinale, Solidago sp., and Tsuga canadensis.

57. The composition according to claim 54, wherein said plant extracts are derived from Zingiber officinale and Solidago sp.

58. The composition according to claim 57, wherein said Solidago sp. is Solidago canadensis, Solidago gigantea, Solidago virgaurea, Solidago hybrida, or any combination thereof.

59. The composition according to claim 57, wherein said Solidago sp. is Solidago virgaurea.

60. The composition according to claim 54, further comprising one or more synthetic MMP-9 and/or cathepsin B inhibitors.

61. The composition according to claim 54, wherein the composition is formulated as a nutraceutical, dietary supplement or naturopathic formulation for oral administration.

62. A method for inhibiting tumour growth or metastasis, comprising administering an effective amount of the composition according to claim 54 to a subject in need thereof.

63. The method according to claim 62, wherein said composition is administered in combination with one or more anti-cancer therapeutics.

64. The method according to claim 63, wherein said anti-cancer therapeutic is a chemotherapeutic drug.

65. The method according to claim 64, wherein said composition potentiates a therapeutic effect of said chemotherapeutic drug.

66. The method according to claim 64, wherein said chemotherapeutic drug is at a sub-optimal dose.

67. The method according to claim 65, wherein said subject is overweight or obese.

68. The method according to claim 62, wherein at least one of said plant extracts is derived from a plant selected from the group of: Zingiber officinale, Solidago sp., and Tsuga canadensis.

69. The method according to claim 62, wherein the composition comprises plant extracts derived from Zingiber officinale and Solidago sp.

70. The method according to claim 69, wherein the Solidago sp. is Solidago canadensis, Solidago gigantea, Solidago virgaurea, Solidago hybrida, or any combination thereof.

71. The method according to claim 69, wherein the Solidago sp. is Solidago virgaurea.

72. A method of inhibiting tumour-induced angiogenesis, comprising administering an effective amount of the composition according to claim 54 to a subject in need thereof.

73. The method according to claim 72, wherein said composition is administered in combination with one or more anti-cancer therapeutics.

74. The method according to claim 73, wherein said anti-cancer therapeutic is a chemotherapeutic drug.

75. The method according to claim 74, wherein said composition potentiates a therapeutic effect of said chemotherapeutic drug.

76. The method according to claim 74, wherein said chemotherapeutic drug is at a sub-optimal dose.

77. The method according to claim 75, wherein said subject is overweight or obese.

78. The method according to claim 72, wherein at least one of said plant extracts is derived from a plant selected from the group of: Zingiber officinale, Solidago sp., and Tsuga canadensis.

79. The method according to claim 72, wherein the composition comprises plant extracts derived from Zingiber officinale and Solidago sp.

80. The method according to claim 79, wherein the Solidago sp. is Solidago canadensis, Solidago gigantea, Solidago virgaurea, Solidago hybrida, or any combination thereof.

81. The method according to claim 79, wherein the Solidago sp. is Solidago virgaurea.

82. A method for treating cancer comprising administering to a subject in need thereof an effective amount of the composition according to claim 54.

83. The method according to claim 82, wherein said composition is administered orally.

84. The method according to claim 82, wherein said composition is administered in combination with one or more anti-cancer therapeutics.

85. The method according to claim 84, wherein said anti-cancer therapeutic is a chemotherapeutic drug.

86. The method according to claim 85, wherein said composition potentiates a therapeutic effect of said chemotherapeutic drug.

87. The method according to claim 85, wherein said chemotherapeutic drug is at a sub-optimal dose.

88. The method according to claim 86, wherein said subject is overweight or obese.

89. The method according to claim 82, wherein at least one of said plant extracts is derived from a plant selected from the group of: Zingiber officinale, Solidago sp., and Tsuga canadensis.

90. The method according to claim 82, wherein the composition comprises plant extracts derived from Zingiber officinale and Solidago sp.

91. The method according to claim 90, wherein the Solidago sp. is Solidago canadensis, Solidago gigantea, Solidago virgaurea, Solidago hybrida, or any combination thereof.

92. The method according to claim 90, wherein the Solidago sp. is Solidago virgaurea.

93. A dietary supplement comprising two or more plant extracts and a physiologically acceptable carrier, wherein each of said plant extracts is derived from Zingiber officinale, Solidago sp., or Tsuga canadensis.

94. The dietary supplement according to claim 93, wherein said plant extracts are derived from Zingiber officinale and Solidago sp.

95. The dietary supplement according to claim 94, wherein the Solidago sp. is Solidago canadensis, Solidago gigantea, Solidago virgaurea, Solidago hybrida, or any combination thereof.

96. The dietary supplement according to claim 94, wherein the Solidago sp. is Solidago virgaurea.

97. The dietary supplement according to claim 93, wherein said two plant extracts are present in a ratio between about 1:3 and about 3:1.

98. The dietary supplement according to claim 93, further comprising one or more phospholipids.

99. The dietary supplement according to claim 98, wherein said phospholipid is lecithin.

100. The dietary supplement according to claim 93, further comprising a plant extract capable of inhibiting MMP-9.

101. The dietary supplement according to claim 100, wherein said plant extract capable of inhibiting MMP-9 is derived from Vaccinium angustifolium.

102. The dietary supplement according to claim 93, wherein said dietary supplement is formulated as a powder or granules for reconstitution in a liquid.

103. A method of treating cancer comprising administering to a subject in need thereof an effective amount of the dietary supplement according to claim 93.

104. The method according to claim 103, wherein said cancer is a solid cancer.

105. The method according to claim 104, wherein said cancer is prostate cancer or colorectal cancer.

106. A kit comprising the composition according to claim 54 and optionally instructions for use.

107. A kit comprising the dietary supplement according to claim 93 and optionally instructions for use.
